SEQUENCE LISTING (13 sequences)

SEQ ID NO: 1 - protein, 5 aa, synthetic construct
  REGION 1..5: note = CDR sequences
HDDIS

SEQ ID NO: 2 - protein, 16 aa, synthetic construct
  REGION 1..16: note = CDR sequences
IISYAGSTYYASWAKG

SEQ ID NO: 3 - protein, 11 aa, synthetic construct
  REGION 1..11: note = CDR sequences
GVPAYSHGGDL

SEQ ID NO: 4 - protein, 13 aa, synthetic construct
  REGION 1..13: note = CDR sequences
TTSQSVYHENWLS

SEQ ID NO: 5 - protein, 7 aa, synthetic construct
  REGION 1..7: note = CDR sequences
WASTLAY

SEQ ID NO: 6 - protein, 10 aa, synthetic construct
  REGION 1..10: note = CDR sequences
AGGYGGGRYA

SEQ ID NO: 7 - protein, 119 aa, synthetic construct
  REGION 1..119: note = CDR sequences
QVQLVESGGGLVQPGGSLRLSCAVSGIDLSHDDISWVRQAPGKGLEWVSIISYAGSTYYASWAKGRLTISKDTSKNQVVLTMTNMDPVDTATYYCARGVPAYSHGGDLWGQGTLVTVSS

SEQ ID NO: 8 - protein, 110 aa, synthetic construct
  REGION 1..110: note = CDR sequences
DIVMTQSPATLSLSPGERATLSCTTSQSVYHENWLSWFQQKPGQPPKLLIYWASTLAYGVPSRFSGSGSGTDFTLTISSLQPEDAATYYCAGGYGGGRYAFGQGTKVEIK

SEQ ID NO: 9 - protein, 447 aa, synthetic construct
  REGION 1..447: note = CDR sequences
QVQLVESGGGLVQPGGSLRLSCAVSGIDLSHDDISWVRQAPGKGLEWVSIISYAGSTYYASWAKGRLTISKDTSKNQVVLTMTNMDPVDTATYYCARGVPAYSHGGDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEYLGGDSVFLFPPKPKDVLMISRTPEVTCVVIDVSHEDPEVKFNWYVDGVEVHNAKTKPREEQYNSTYRVVSVLPVLHRDWLNGKEYKCKVSNKALPKPIEKTISKAKGQRREPQVYTLPPSREEMTKNQVSLTCLVKGFYPSDIAVEWESNGQPENNYKTTPPVLDSDGSFFLYSKLTVDKSRWQQGNVFSCSVLHEALHAHTTRKELSLSP

SEQ ID NO: 10 - protein, 217 aa, synthetic construct
  REGION 1..217: note = CDR sequences
DIVMTQSPATLSLSPGERATLSCTTSQSVYHENWLSWFQQKPGQPPKLLIYWASTLAYGVPSRFSGSGSGTDFTLTISSLQPEDAATYYCAGGYGGGRYAFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 375 aa, Homo sapiens
MQKLQLCVYIYLFMLIVAGPVDLNENSEQKENVEKEGLCNACTWRQNTKSSRIEAIKIQILSKLRLETAPNISKDVIRQLLPKAPPLRELIDQYDVQRDDSSDGSLEDDDYHATTETIITMPTESDFLMQVDGKPKCCFFKFSSKIQYNKVVKAQLWIYLRPVETPTTVFVQILRLIKPMKDGTRYTGIRSLKLDMNPGTGIWQSIDVKTVLQNWLKQPESNLGIEIKALDENGHDLAVTFPGPGEDGLNPFLEVKVTDTPKRSRRDFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 12 - protein, 242 aa, Homo sapiens
ENSEQKENVEKEGLCNACTWRQNTKSSRIEAIKIQILSKLRLETAPNISKDVIRQLLPKAPPLRELIDQYDVQRDDSSDGSLEDDDYHATTETIITMPTESDFLMQVDGKPKCCFFKFSSKIQYNKVVKAQLWIYLRPVETPTTVFVQILRLIKPMKDGTRYTGIRSLKLDMNPGTGIWQSIDVKTVLQNWLKQPESNLGIEIKALDENGHDLAVTFPGPGEDGLNPFLEVKVTDTPKRSRR

SEQ ID NO: 13 - protein, 266 aa, Homo sapiens
MQKLQLCVYIYLFMLIVAGPVDLNENSEQKENVEKEGLCNACTWRQNTKSSRIEAIKIQILSKLRLETAPNISKDVIRQLLPKAPPLRELIDQYDVQRDDSSDGSLEDDDYHATTETIITMPTESDFLMQVDGKPKCCFFKFSSKIQYNKVVKAQLWIYLRPVETPTTVFVQILRLIKPMKDGTRYTGIRSLKLDMNPGTGIWQSIDVKTVLQNWLKQPESNLGIEIKALDENGHDLAVTFPGPGEDGLNPFLEVKVTDTPKRSRR

(19) United States
(12) Patent Application Publication
(10) Pub. No.: US 2024/0358704 A1
(43) Pub. Date: Oct. 31, 2024

(54) COMBINED ADMINISTRATION

(71) Applicant: Hoffmann-La Roche Inc., Little Falls, NJ (US)

(72) Inventors: Nicole HELLBACH, Bad Krozingen (DE); Heidemarie KLETZL, Basel (CH); Friedrich METZGER, Freiburg (DE); Renata SICILIANI SCALCO, Basel (CH)

(73) Assignee: Hoffmann-La Roche Inc., Little Falls, NJ (US)

(21) Appl. No.: 18/627,727

(22) Filed: Apr. 5, 2024

Related U.S. Application Data
(63) Continuation of application No. PCT/EP2022/077494, filed on Oct. 4, 2022.

(30) Foreign Application Priority Data
Oct. 6, 2021 (EP) 21201211.6

(51) Int. Cl. A61K 31/519; A61K 9/00; A61K 9/08; A61K 39/00; A61K 39/395; A61P 21/00

(52) U.S. Cl. CPC A61K 31/519; A61K 9/0053; A61K 9/0095; A61K 9/08; A61K 39/3955; A61P 21/00; A61K 2039/545

(57) ABSTRACT

The invention relates to 7-(4,7-diazaspiro[2.5]octan-7-yl)-2-(2,8-dimethylimidazo[1,2-b]pyridazin-6-yl)pyrido[1,2-a]pyrimidin-4-one, also known as risdiplam, for use in the treatment of spinal muscular atrophy (SMA) with GYM329, its pharmaceutical composition to be used in the treatment of SMA, its methods of treatment thereof.

### CROSS REFERENCE TO RELATED APPLICATIONS

This application is a continuation of International Application No. PCT/EP2022/077494, filed Oct. 4, 2022, which claims priority to EP Application No. 21201211.6, filed Oct. 6, 2021, the disclosures of each of which are incorporated herein by reference in its entirety.

### SEQUENCE LISTING

This application contains a Sequence Listing which has been submitted electronically in XML format and is hereby incorporated by reference in its entirety. Said XML copy, created on Mar. 26, 2024, is named “P37143 sequence listing.xml” and is 17,671 bytes in size.

### BRIEF SUMMARY

The invention relates to 7-(4,7-diazaspiro[2.5]octan-7-yl)-2-(2,8-dimethylimidazo[1,2-b]pyridazin-6-yl)pyrido[1,2-a]pyrimidin-4-one, also known as risdiplam, for use in the treatment of spinal muscular atrophy (SMA) with GYM329, its pharmaceutical composition to be used in the treatment of SMA, its methods of treatment thereof.

The present invention is directed to the combined administration of risdiplam and GYM329, a myostatin inhibitor. In another embodiment the present invention is risdiplam for use in treating spinal muscular atrophy in combination with GYM329.

An isolated antibody that binds to latent myostatin and does not bind to mature myostatin, wherein the antibody blocks the non-proteolytic, spontaneous release of mature myostatin from latent myostatin and inhibits activation of myostatin wherein the antibody comprises six complementary determining regions (CDRs): CDRH1, CDRH2, CDRH3, CDRL1, CDRL2 and CDRL3 wherein CDRH1 comprises a sequence set forth in SEQ ID 1, CDRH2 comprises a sequence set forth in SEQ ID 2, CDRH3 comprises a sequence set forth in SEQ ID 3, CDRL1 comprises a sequence set forth in SEQ ID 4, CDRL2 comprises a sequence set forth in SEQ ID 5 and CDRL3 comprises a sequence set forth in SEQ ID 6 with risdiplam for use in treating SMA.

An isolated antibody that binds to latent myostatin and does not bind to mature myostatin, wherein the antibody blocks the non-proteolytic, spontaneous release of mature myostatin from latent myostatin and inhibits activation of myostatin wherein the antibody comprises a VH having at least 90% sequence identity to the amino acid sequence of SEQ ID NO: 7 and a VL having at least 90% sequence identity to the amino acid sequence of SEQ ID NO: 8 with risdiplam for use in treating SMA.

An isolated antibody that binds to latent myostatin and does not bind to mature myostatin, wherein the antibody blocks the non-proteolytic, spontaneous release of mature myostatin from latent myostatin and inhibits activation of myostatin wherein the antibody comprises a heavy chain region comprising an amino acid sequence of SEQ ID 9 and light chain region comprising an amino acid sequence of SEQ ID 10 with risdiplam for use in treating SMA.

Spinal muscular atrophy (SMA), in its broadest sense, describes a collection of inherited and acquired central nervous system (CNS) diseases characterized by progressive motor neuron loss in the spinal cord and brainstem causing muscle weakness and muscle atrophy. The most common form of SMA is caused by mutations in the Survival Motor Neuron (SMN) gene and manifests over a wide range of severity affecting infants through adults (Crawford and Pardo, Neurobiol. Dis., 1996, 3:97).

Infantile SMA is the most severe form of this neurodegenerative disorder. Symptoms include muscle weakness, poor muscle tone, weak cry, limpness or a tendency to flop, difficulty sucking or swallowing, accumulation of secretions in the lungs or throat, feeding difficulties, and increased susceptibility to respiratory tract infections. The legs tend to be weaker than the arms and developmental milestones, such as lifting the head or sitting up, cannot be reached. In general, the earlier the symptoms appear, the shorter the lifespan. As the motor neuron cells deteriorate, symptoms appear shortly afterward. The severe forms of the disease are fatal and all forms have no known cure. The course of SMA is directly related to the rate of motor neuron cell deterioration and the resulting severity of weakness. Infants with a severe form of SMA frequently succumb to respiratory disease due to weakness in the muscles that support breathing. Children with milder forms of SMA live much longer, although they may need extensive medical support, especially those at the more severe end of the spectrum. The clinical spectrum of SMA disorders has been divided into the following five groups.
1) Type 0 SMA (In Utero SMA) is the most severe form of the disease and begins before birth. Usually, the first symptom of Type 0 SMA is reduced movement of the fetus that can first be observed between 30 and 36 weeks of pregnancy. After birth, these newborns have little movement and have difficulties with swallowing and breathing.
2) Type 1 SMA (Infantile SMA or Werdnig-Hoffmann disease) presents symptoms between 0 and 6 months. This form of SMA is also very severe. Patients never achieve the ability to sit, and death usually occurs within the first 2 years without ventilatory support.
3) Type 2 SMA (Intermediate SMA) has an age of onset at 7-18 months. Patients achieve the ability to sit unsupported, but never stand or walk unaided. Prognosis in this group is largely dependent on the degree of respiratory involvement.
4) Type 3 SMA (Juvenile SMA or Kugelberg-Welander disease) is generally diagnosed after 18 months. Type 3 SMA individuals are able to walk independently at some point during their disease course but often become wheelchair-bound during youth or adulthood.
5) Type 4 SMA (Adult onset SMA). Weakness usually begins in late adolescence in the tongue, hands, or feet, then progresses to other areas of the body. The course of adult SMA is much slower and has little or no impact on life expectancy.

The SMN gene has been mapped by linkage analysis to a complex region in chromosome 5q. In humans, this region contains an approximately 500 thousand base pairs (kb) inverted duplication resulting in two nearly identical copies of the SMN gene. SMA is caused by an inactivating mutation or deletion of the telomeric copy of the gene (SMN1) in both chromosomes, resulting in the loss of SMN1 gene function. However, all patients retain the centromeric copy of the gene (SMN2), and the copy number of the SMN2 gene in SMA patients generally correlates inversely with the disease severity; i.e., patients with less severe SMA have more copies of SMN2. Nevertheless, SMN2 is unable to compensate completely for the loss of SMN1 function due to alternative splicing of exon 7 caused by a translationally silent C to T mutation in exon 7. As a result, the majority of transcripts produced from SMN2 lack exon 7 (A7 SMN2), and encode a truncated SMN protein that has an impaired function and is rapidly degraded.

The SMN protein is thought to play a role in RNA processing and metabolism, having a well characterized function of mediating the assembly of a specific class of RNA-protein complexes termed snRNPs. SMN may have other functions in motor neurons, however its role in preventing the selective degeneration of motor neurons is not well established.

In most cases, SMA is diagnosed based on clinical symptoms and by the presence of at least one copy of the SMN1 gene test. However, in approximately 5% of cases SMA is caused by mutation in genes other than the inactivation of SMN 1, some known and others not yet defined. In some cases, when the SMN 1 gene test is not feasible or does not show any abnormality, other tests such as an electromyography (EMG) or muscle biopsy may be indicated.

Several mouse models of SMA have been developed. In particular, the SMN delta exon 7 (A7 SMN) model (Le et al., Hum. Mol. Genet., 2005, 14:845) carries both the SMN2 gene and several copies of the A7 SMN2 cDNA and recapitulates many of the phenotypic features of Type 1 SMA. The A7 SMN model can be used for both SMN2 expression studies as well as the evaluation of motor function and survival. The C/C-allele mouse model (Jackson Laboratory strain #008714, The Jackson Laboratory, Bar Harbor, ME) provides a less severe SMA disease model, with mice having reduced levels of both SMN2 full length (FL SMN2) mRNA and SMN protein. The C/C-allele mouse phenotype has the SMN2 gene and a hybrid mSMN1-SMN2 gene that undergoes alternative splicing, but does not have overt muscle weakness. The C/C-allele mouse model is used for SMN2 expression studies.

As a result of improved understanding of the genetic basis and pathophysiology of SMA, several strategies for treatment have been explored, which have led to the approval of three treatments Nusinersen (Spinraza®), an intrathecally delivered antisense oligonucleotide (ASO) targeting the SMN2 gene, Onasemnogene abeparvovec-xioi (Zolgensma®) an IV administered adeno-associated viral vector-based gene therapy that delivers a copy of the survival of motor neuron 1 (SMN1) gene, and Risdiplam (Evrysdi®), an oral survival of motor neuron 2 (SMN2)-splicing modifier. Those available treatments differ by their mechanisms of actions on the disease and their means of administration. Risdiplam is the only oral treatment available for SMA patients. Risdiplam has been approved in most major market.

Myostatin, referred to as growth differentiation factor-8 (GDF8), is a secreted protein and is a member of the transforming growth factor-beta (TGF-beta) superfamily of proteins. Members of this superfamily possess growth-regulatory and morphogenetic properties (see, e.g., NPL1, NPL2, and PTL1). Myostatin is expressed primarily in the developing and adult skeletal muscle and functions as a negative regulator of muscle growth. Systemic overexpression of myostatin in adult mice leads to muscle wasting (see, e.g., NPL3) while, conversely, a myostatin knockout mouse is characterized by hypertrophy and hyperplasia of the skeletal muscle resulting in two- to threefold greater muscle mass than their wild type littermates (see, e.g., NPL4).

Like other members of the TGF-beta family, myostatin is synthesized as a large precursor protein containing an N-terminal propeptide domain, and a C-terminal domain considered as the active molecule (see, e.g., NPL5; PTL2). Two molecules of myostatin precursor are covalently linked via a single disulfide bond present in the C-terminal growth factor domain. Active mature myostatin (disulfide-bonded homodimer consisting of the C-terminal growth factor domain) is liberated from myostatin precursor through multiple steps of proteolytic processing. In the first step of the myostatin activation pathway, a peptide bond between the N-terminal propeptide domain and the C-terminal growth factor domain, Arg266-Asp267, is cleaved by a furin-type proprotein convertase in both chains of the homodimeric precursor. But the resulting three peptides (two propeptides and one mature myostatin (i.e., a disulfide-bonded homodimer consisting of the growth factor domains)) remain associated, forming a noncovalent inactive complex that is referred to as “latent myostatin.” Mature myostatin can then be liberated from latent myostatin through degradation of the propeptide. Members of the bone morphogenetic protein 1 (BMP1) family of metalloproteinases cleave a single peptide bond within the propeptide, Arg98-Asp99, with concomitant release of mature, active myostatin, a homodimer (see, e.g., NPL6). Moreover, the latent myostatin can be activated in vitro by dissociating the complex with either acid or heat treatment as well (see, e.g., NPL7).

Myostatin exerts its effects through a transmembrane serine/threonine kinase heterotetramer receptor family, activation of which enhances receptor transphosphorylation, leading to the stimulation of serine/threonine kinase activity. It has been shown that the myostatin pathway involves an active myostatin dimer binding to the activin receptor type IIB (ActRIIB) with high affinity, which then recruits and activates the transphosphorylation of the low affinity receptor, the activin-like kinase 4 (ALK4) or activin-like kinase 5 (ALK5). It has also been shown that the proteins Smad 2 and Smad 3 are subsequently activated and form complexes with Smad 4, which are then translocated to the nucleus for the activation of target gene transcription. It has been demonstrated that ActRIIB is able to mediate the influence of myostatin in vivo, as expression of a dominant negative form of ActRIIB in mice mimics myostatin gene knockout (see, e.g., NPL8).

A number of disorders or conditions are associated with muscle wasting (i.e., loss of or functional impairment of muscle tissue), such as muscular dystrophy (MD; including Duchenne muscular dystrophy), amyotrophic lateral sclerosis (ALS), muscle atrophy, Spinal muscular atrophy (SMA); Spinal muscular atrophy with respiratory distress type 1; Stiff person syndrome; Troyer syndrome; Guillain-Barre syndrome, organ atrophy, frailty, congestive obstructive pulmonary disease (COPD), sarcopenia, and cachexia resulting from cancer or other disorders, as well as renal disease, cardiac failure or disease, and liver disease. Patients will benefit from an increase in muscle mass and/or muscle strength; however, there are presently limited treatments available for these disorders. Thus, due to its role as a negative regulator of skeletal muscle growth, myostatin becomes a desirable target for therapeutic or prophylactic intervention for such disorders or conditions, or for monitoring the progression of such disorders or conditions. In particular, agents that inhibit the activity of myostatin may be therapeutically beneficial.

Inhibition of myostatin expression leads to both muscle hypertrophy and hyperplasia (NPL9). Myostatin negatively regulates muscle regeneration after injury and lack of myostatin in myostatin null mice results in accelerated muscle regeneration (see, e.g., NPL10). Anti-myostatin (GDF8) antibodies described in, e.g., PTL3, PTL4, PTL5, PTL6, and PTL7, and PTL8, PTL9, and PTL10 have been shown to bind to myostatin and inhibit myostatin activity in vitro and in vivo, including myostatin activity associated with the negative regulation of skeletal muscle mass. Myostatin-neutralizing antibodies increase body weight, skeletal muscle mass, and muscle size and strength in the skeletal muscle of wild type mice (see, e.g., NPL11) and the mdx mice, a model for muscular dystrophy (see, e.g., NPL12; NPL13). However, these prior art antibodies are all specific for mature myostatin but not for latent myostatin, and the strategies described for inhibiting myostatin activity have utilized antibodies that can bind to and neutralize mature myostatin.

Antibodies are drawing attention as pharmaceuticals since they are highly stable in blood and have few side effects (see, e.g., NPL14 and NPL15). Almost all therapeutic antibodies currently on the market are antibodies of the human IgG1 subclass. One of the known functions of IgG class antibodies is antibody-dependent cell-mediated cytotoxicity (hereinafter denoted as ADCC activity) (see, e.g., NPL16). For an antibody to exhibit ADCC activity, the antibody Fc region must bind to an Fc gamma receptor (hereinafter denoted as Fc gamma R) which is an antibody-binding receptor present on the surface of effector cells such as killer cells, natural killer cells, and activated macrophages.

### BRIEF DESCRIPTION OF THE SEVERAL VIEWS OF THE DRAWINGS

FIG. 1 Study Design. This figure depicts the groups examined in this study, including A7 mice treated with GYM329 or vehicle alone or in combination with SMN-upregulation and WT littermates.

FIG. 2 Growth Curves. WT: n=11, SMA Vehicle: n=10, SMA GYM329: n=11. WT: wild type littermates; SMA Vehicle: delta 7 mice+low dose SMN-C1+Vehicle; SMA GYM329: delta 7 mice+low dose SMN-C1+GYM329.

FIG. 3 Body Weight and Muscle Weights at PND52. A. Body weight. B. Gastrocnemius weight. C. Soleus weight. D. TA weight. E. EDL weight. F. Masseter weight. WT: n=11, SMA Vehicle: n=10, SMA GYM329: n=11. WT: wild type littermates; SMA Vehicle: delta 7 mice+low dose SMN-C1+Vehicle; SMA GYM329: delta 7 mice+low dose SMN-C1+GYM329.

FIG. 4 Plantarflexor Muscle Function at PND52. Maximum torque. WT: n=11, SMA Vehicle: n=10, SMA GYM329: n=11. WT: wild type littermates; SMA Vehicle: delta 7 mice+low dose SMN-C1+Vehicle; SMA GYM329: delta 7 mice+low dose SMN-C1+GYM329.

FIG. 5 Masseter Muscle Function at PND52. Maximum force. WT: n=11, SMA Vehicle: n=10, SMA GYM329: n=11. WT: wild type littermates; SMA Vehicle: delta 7 mice+low dose SMN-C1+Vehicle; SMA GYM329: delta 7 mice+low dose SMN-C1+GYM329.

FIG. 6 Plantarflexor Fiber Types and Cross-Sectional Area (CSA). A. Muscle fiber types. B. Mean fiber CSA. C. Type I fiber CSA. D. Type IIA fiber CSA. E. Type IIB fiber CSA. F. Type IIX fiber CSA. WT: n=11, SMA Vehicle: n=10, SMA GYM329: n=11. WT: wild type littermates; SMA Vehicle: delta 7 mice+low dose SMN-C1+Vehicle; SMA GYM329: delta 7 mice+low dose SMN-C1+GYM329.

FIG. 7 Cortical Bone Parameters: Tibia. A. Cross-sectional thickness. B. Mean total cross-sectional tissue area. C. Mean total cross-sectional tissue perimeter. D. Mean total cross-section bone area. E. Mean total cross-section bone perimeter. F. Cortical porosity. WT: n=10, SMA Vehicle: n=8, SMA GYM329: n=10. WT: wild type littermates; SMA Vehicle: delta 7 mice+low dose SMN-C1+Vehicle; SMA GYM329: delta 7 mice+low dose SMN-C1+GYM329.

FIG. 8 Trabecular Bone Parameters: Tibia. A. Bone volume. B. Trabecular thickness. C. Trabecular number. D. Trabecular separation. WT: n=11, SMA Vehicle: n=8, SMA GYM329: n=10. WT: wild type littermates; SMA Vehicle: delta 7 mice+low dose SMN-C1+Vehicle; SMA GYM329: delta 7 mice+low dose SMN-C1+GYM329.

FIG. 9 Growth Curves. WT: n=11, SMA Vehicle: n=10, SMA GYM329: n=10. WT: wild type littermates; SMA Vehicle: delta 7 mice+low to high dose SMN-C1+Vehicle; SMA GYM329: delta 7 mice+low to high dose SMN-C1+GYM329.

FIG. 10 Body Weight and Muscle Weights at PND52. A. Body weight. B. Gastrocnemius weight. C. Soleus weight. D. TA weight. E. EDL weight. F. Masseter weight. WT: n=11, SMA Vehicle: n=10, SMA GYM329: n=10. WT: wild type littermates; SMA Vehicle: delta 7 mice+low to high dose SMN-C1+Vehicle; SMA GYM329: delta 7 mice+low to high dose SMN-C1+GYM329.

FIG. 11 Plantarflexor Muscle Function at PND52. Maximum torque. WT: n=11, SMA Vehicle: n=10, SMA GYM329: n=10. WT: wild type littermates; SMA Vehicle: delta 7 mice+low to high dose SMN-C1+Vehicle; SMA GYM329: delta 7 mice+low to high dose SMN-C1+GYM329.

FIG. 12 Masseter Function at PND52. Maximum force. WT: n=11, SMA Vehicle: n=10, SMA GYM329: n=10. WT: wild type littermates; SMA Vehicle: delta 7 mice+low to high dose SMN-C1+Vehicle; SMA GYM329: delta 7 mice+low to high dose SMN-C1+GYM329.

FIG. 13 Plantarflexor Fiber Types and Cross-Sectional Area (CSA). A. Muscle fiber types. B. Mean fiber CSA. C. Type I fiber CSA. D. Type IIA fiber CSA. E. Type IIB fiber CSA. F. Type IIX fiber CSA. WT: n=11, SMA Vehicle: n=10, SMA GYM329: n=10. WT: wild type littermates; SMA Vehicle: delta 7 mice+low to high dose SMN-C1+Vehicle; SMA GYM329: delta 7 mice+low to high dose SMN-C1+GYM329.

FIG. 14 Cortical Bone Parameters: Tibia. A. Cross-sectional thickness. B. Mean total cross-sectional tissue area. C. Mean total cross-sectional tissue perimeter. D. Mean total cross-section bone area. E. Mean total cross-section bone perimeter. F. Cortical porosity. WT: n=10, SMA Vehicle: n=9, SMA GYM329: n=9. WT: wild type littermates; SMA Vehicle: delta 7 mice+low to high dose SMN-C1+Vehicle; SMA GYM329: delta 7 mice+low to high dose SMN-C1+GYM329. * p<0.05 vs. SMA Vehicle.

FIG. 15 Trabecular Bone Parameters: Tibia. A. Bone volume. B. Trabecular thickness. C. Trabecular number. D. Trabecular separation. WT: n=11, SMA Vehicle: n=9, SMA GYM329: n=9. WT: wild type littermates; SMA Vehicle: delta 7 mice+low to high dose SMN-C1+Vehicle; SMA GYM329: delta 7 mice+low to high dose SMN-C1+GYM329.

FIG. 16 Growth Curves. WT: n=11, SMA Vehicle: n=15, SMA GYM329: n=14. WT: wild type littermates; SMA Vehicle: delta 7 mice+high dose SMN-C1+Vehicle; SMA GYM329: delta 7 mice+high dose SMN-C1+GYM329.

FIG. 17 Body Weight and Muscle Weights at PND52. A. Body weight. B. Gastrocnemius weight. C. Soleus weight. D. TA weight. E. EDL weight. F. Masseter weight. WT: n=11, SMA Vehicle: n=15, SMA GYM329: n=14. WT: wild type littermates; SMA Vehicle: delta 7 mice+high dose SMN-C1+Vehicle; SMA GYM329: delta 7 mice+high dose SMN-C1+GYM329. * p<0.05 vs. SMA Vehicle.

FIG. 18 Plantarflexor Muscle Function at PND52. Maximum torque. WT: n=11, SMA Vehicle: n=15, SMA GYM329: n=14. WT: wild type littermates; SMA Vehicle: delta 7 mice+high dose SMN-C1+Vehicle; SMA GYM329: delta 7 mice+high dose SMN-C1+GYM329.

FIG. 19 Masseter Function at PND52. Maximum force. WT: n=11, SMA Vehicle: n=15, SMA GYM329: n=14. WT: wild type littermates; SMA Vehicle: delta 7 mice+high dose SMN-C1+Vehicle; SMA GYM329: delta 7 mice+high dose SMN-C1+GYM329. * p<0.05 SMA GYM329 vs. SMA Vehicle.

FIG. 20 Plantarflexor Fiber Types and Cross-Sectional Area (CSA). A. Muscle fiber types. B. Mean fiber CSA. C. Type I fiber CSA. D. Type IIA fiber CSA. E. Type IIB fiber CSA. F. Type IIX fiber CSA. WT: n=11, SMA Vehicle: n=15, SMA GYM329: n=14. WT: wild type littermates; SMA Vehicle: delta 7 mice+high dose SMN-C1+Vehicle; SMA GYM329: delta 7 mice+high dose SMN-C1+GYM329. * p<0.05 vs. SMA Vehicle.

FIG. 21 Cortical Bone Parameters: Tibia. A. Mean total cross-sectional tissue perimeter. B. Mean total cross-sectional tissue area. C. Mean tissue cross-sectional bone perimeter. D. Mean total cross-section bone area. E. Cross-sectional thickness. F. Cortical porosity. WT: n=10, SMA Vehicle: n=10, SMA GYM329: n=13. WT: wild type littermates; SMA Vehicle: delta 7 mice+high dose SMN-C1+Vehicle; SMA GYM329: delta 7 mice+high dose SMN-C1+GYM329. * p<0.05 vs. SMA Vehicle.

FIG. 22 Trabecular Bone Parameters: Tibia. A. Percent bone volume. B. Trabecular thickness. C. Trabecular number. D. Trabecular separation. WT: n=11, SMA Vehicle: n=14, SMA GYM329: n=14. WT: wild type littermates; SMA Vehicle: delta 7 mice+high dose SMN-C1+Vehicle; SMA GYM329: delta 7 mice+high dose SMN-C1+GYM329. * p<0.05 vs. SMA Vehicle.

FIG. 23 Clinical Study Scheme of Part 1; OLE=Open-Label Extension; ^{a} Participants naïve to risdiplam will be treated with risdiplam for at least 8 weeks in the run-in period before randomization for the 24-week double-blind period. Participants treated with risdiplam for at least 8 continuous weeks immediately prior to joining this study may be randomized to combination therapy immediately or will join the run-in period and continue to receive risdiplam monotherapy until randomization. ^{b} Age at screening. ° Blinded GYM329 or GYM329-matched placebo. ^{d} If a participant reaches the end of the 24-week double-blind treatment period and the pivotal dose has not yet been determined, the participant will receive GYM329 at the dose of their respective treatment cohort until the pivotal dose has been decided. Once the pivotal dose is selected, participants will then be switched to this pivotal dose. FIG. 24 Clinical Study Scheme of Part 2, OLE=open-label extension; a Age at screening; b GYM329 at the dose selected in Part 1 (pivotal dose); c Blinded GYM329 or GYM329-matched placebo.

### DETAILED DESCRIPTION

All publications, patent applications, patents and other references mentioned herein are incorporated by reference in their entirety.

The nomenclature used in the present application is based on IPUAC systematic nomenclature, unless indicated otherwise.

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), and March, Advanced Organic Chemistry Reactions, Mechanisms and Structure 4th ed., John Wiley & Sons (New York, N.Y. 1992), provide one skilled in the art with a general guide to many of the terms used in the present application. All references cited herein, including patent applications and publications, are incorporated by reference in their entirety.

For purposes of interpreting this specification, the following definitions will apply and whenever appropriate, terms used in the singular will also include the plural and vice versa. It is to be understood that the terminology used herein is for the purpose of describing particular embodiments only, and is not intended to be limiting. Unless otherwise stated, the following terms used in the specification and claims have the meanings given below:

An “individual” or “subject”, used interchangeably, is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human. In a particular embodiment of the invention the subject is a human with spinal muscular atrophy (SMA). In another specific embodiment, the subject is a human with SMA caused by an inactivating mutation or deletion in the SMN1 gene on both chromosomes, resulting in a loss of SMN1 gene function.

The term “spinal muscular atrophy” (or SMA) relates to a disease caused by an inactivating mutation or deletion in the SMN1 gene on both chromosomes, resulting in a loss of SMN1 gene function. Symptoms of SMA—depending on the type of SMA—include muscle weakness, poor muscle tone, weak cry, weak cough, limpness or a tendency to flop, difficulty sucking or swallowing, difficulty breathing, accumulation of secretions in the lungs or throat, clenched fists with sweaty hand, flickering/vibrating of the tongue, head often tilted to one side, even when lying down, legs that tend to be weaker than the arms, legs frequently assuming a “frog legs” position, feeding difficulties, increased susceptibility to respiratory tract infections, bowel/bladder weakness, lower-than-normal weight, inability to sit without support, failure to walk, failure to crawl, and hypotonia, areflexia, and multiple congenital contractures (arthrogryposis) associated with loss of anterior horn cells.

The term “treating spinal muscular atrophy (SMA)” or “treatment of spinal muscular atrophy (SMA)” includes one or more of the following effects: (i) reduction or amelioration of the severity of SMA; (ii) delay of the onset of SMA; (iii) inhibition of the progression of SMA; (iv) reduction of hospitalization of a subject; (v) reduction of hospitalization length for a subject; (vi) increase of the survival of a subject; (vii) improvement of the quality of life of a subject; (viii) reduction of the number of symptoms associated with SMA; (ix) reduction of or amelioration of the severity of one or more symptoms associated with SMA; (x) reduction of the duration of a symptom associated with SMA; (xi) prevention of the recurrence of a symptom associated with SMA; (xii) inhibition of the development or onset of a symptom of SMA; (xiii) inhibition of the progression of a symptom associated with SMA; and/or (xiv) stabilization of the number of symptoms associated with SMA. More particular, “treating SMA” denotes one or more of the following beneficial effects: (i) a reduction in the loss of muscle strength; (ii) an increase in muscle strength; (iii) a reduction in muscle atrophy; (iv) a reduction in the loss of motor function; (v) an increase in motor neurons; (vii) a reduction in the loss of motor neurons; (viii) protection of SMN deficient motor neurons from degeneration; (ix) an increase in motor function; (x) an increase in pulmonary function; and/or (xi) a reduction in the loss of pulmonary function; and/or (xii) a stabilization in motor function.

In detail, “treating SMA” results in the functional ability or helps retain the functional ability for a human infant or a human toddler to sit up unaided or for a human infant, a human toddler, a human child or a human adult to stand up unaided, to walk unaided, to run unaided, to breathe unaided, to turn during sleep unaided, or to swallow unaided.

The term “mg/kg” refers to the dose in milligram of 7-(4,7-diazaspiro[2.5]octan-7-yl)-2-(2,8-dimethylimidazo[1,2-b]pyridazin-6-yl)pyrido[1,2-a]pyrimidin-4-one being used per kilogram of body weight of the subject to be treated. For example, 0.25 mg/kg means a dose of 0.25 milligram of 7-(4,7-diazaspiro[2.5]octan-7-yl)-2-(2,8-dimethylimidazo[1,2-b]pyridazin-6-yl)pyrido[1,2-a]pyrimidin-4-one per kilogram of body weight of the patient to be treated.

The term “patient” refers to a human (such as a male or female human) who has been diagnosed with SMA.

The term “active pharmaceutical ingredient” (or “API”) denotes the compound or molecule in a pharmaceutical composition that has a particular biological activity.

The terms “pharmaceutically acceptable excipient”, “pharmaceutically acceptable carrier” and “therapeutically inert excipient” can be used interchangeably and denote any pharmaceutically acceptable ingredient in a pharmaceutical composition having no therapeutic activity and being non-toxic to the subject administered, such as disintegrators, binders, fillers, solvents, buffers, tonicity agents, stabilizers, antioxidants, surfactants, carriers, diluents or lubricants used in formulating pharmaceutical products.

The term “pharmaceutical composition” refers to a preparation which is in such form as to permit the biological activity of an active ingredient contained therein to be effective, and which contains no additional components which are unacceptably toxic to a subject to which the composition would be administered. The term “pharmaceutically acceptable” denotes an attribute of a material which is useful in preparing a pharmaceutical composition that is generally safe, non-toxic, and neither biologically nor otherwise undesirable and is acceptable for veterinary as well as human pharmaceutical use.

“risdiplam” or “7-(4,7-diazaspiro[2.5]octan-7-yl)-2-(2,8-dimethylimidazo[1,2-b]pyridazin-6-yl)pyrido[1,2-a]pyrimidin-4-one”, used interchangeably, according to the present invention refers to a compound of formula (I),

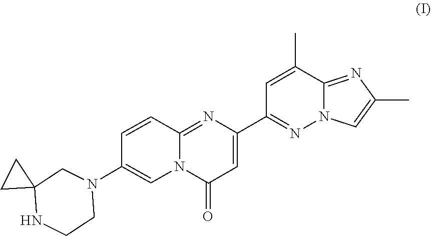 [SMILES: Cc1cn2nc(-c3cc(=O)n4cc(N5CCNC6(CC6)C5)ccc4n3)cc(C)c2n1]

also known as Evrysdi®, RG7916, R07034067, CAS Number 1825352-65-5. Risdiplam according to the present invention can be referred to its chemical name, chemical structure or any alternative reference as herewith mentioned. In particular, risdiplam can be used interchangeably with its chemical name 7-(4,7-diazaspiro[2.5]octan-7-yl)-2-(2,8-dimethylimidazo[1,2-b]pyridazin-6-yl)pyrido[1,2-a]pyrimidin-4-one. Methods of making and using the compound are described in EP3143025 A1. Methods of making and using the pharmaceutical composition are described in WO2017080967 A1 and WO202079203.

The term “Cₘₐₓ” (expressed in units of ng/mL) means maximum observed plasma concentration.

The term “Tₘₐₓ” (expressed in units of hours, or as a median number of hours for Tₘₐₓ in the study population) means the observed time to reach Cₘₐₓ following drug administration; if it occurs at more than one time point Tₘₐₓ is defined as the first time point with this value.

The term “AUC_{T0-24 h}” (expressed in units of ng h/mL) means the area under the plasma concentration time curve (AUC).

The term “buffer” or “buffer system” denotes a pharmaceutically acceptable excipient or excipient mixture, which stabilizes the pH of a pharmaceutical preparation. Suitable buffers are well known in the art and can be found in the literature. Particular pharmaceutically acceptable buffers comprise citric buffer, malate buffer, maleate buffer, or tartrate buffer, most particularly tartrate buffer. Particular buffer systems of the invention combinations of organic acid and selected salts thereof, e.g. tribasic sodium citrate and citric acid, malic acid and sodium malate, potassium sodium tartrate and tartaric acid, or disodium tartrate and tartaric acid, particularly potassium sodium tartrate and tartaric acid. Alternatively, the organic acid (particularly tartaric acid) can be employed alone as “acidifier” instead of the combination of acid and the corresponding salt. Independently from the buffer used, the pH can be adjusted with an acid or a base known in the art, e.g., hydrochloric acid, acetic acid, phosphoric acid, sulfuric acid and citric acid, sodium hydroxide and potassium hydroxide. Particular acidifier is tartaric acid.

The term “antioxidant” denotes pharmaceutically acceptable excipients, which prevent oxidation of the active pharmaceutical ingredient. Antioxidants comprise ascorbic acid, glutathione, cysteine, methionine, vitamin E TPGS, EDTA.

The term “therapeutically effective amount,” as used herein, refers to an amount of a compound sufficient to treat, ameliorate, or prevent the identified disease or condition, or to exhibit a detectable therapeutic, prophylactic, or inhibitory effect. The effect can be detected by, for example, an improvement in clinical condition, or reduction in symptoms. The precise effective amount for a subject will depend upon the subject's body weight, size, and health; the nature and extent of the condition; and the therapeutic or combination of therapeutics selected for administration. Where a drug has been approved by the U.S. Food and Drug Administration (FDA), a “therapeutically effective amount” refers to the dosage approved by the FDA or its counterpart foreign agency for treatment of the identified disease or condition.

As used herein, a patient “in need of risdiplam therapy” is a patient who would benefit from administration of risdiplam. The patient may be suffering from any disease or condition for which risdiplam therapy may be useful in ameliorating symptoms. Risdiplam is being developed for treating spinal muscular atrophy.

As used herein, a patient “in need of GYM329 therapy” (or “in need of an anti-myostatin antibody therapy”) is a patient who would benefit from administration of GYM329. The patient may be suffering from any disease or condition for which risdiplam therapy may be useful in ameliorating symptoms. GYM329 is being developed for treating spinal muscular atrophy in combination with risdiplam.

“GYM329” also known R07204239 according to the present invention refers to an “anti-myostatin antibody”, wherein the antibody comprises six complementary determining regions (CDRs): CDRH1, CDRH2, CDRH3, CDRL1, CDRL2 and CDRL3 wherein CDRH1 comprises a sequence set forth in SEQ ID 1, CDRH2 comprises a sequence set forth in SEQ ID 2, CDRH3 comprises a sequence set forth in SEQ ID 3, CDRL1 comprises a sequence set forth in SEQ ID 4, CDRL2 comprises a sequence set forth in SEQ ID 5 and CDRL3 comprises a sequence set forth in SEQ ID 6. GYM329 can also be defined by a heavy chain variable region comprising an amino acid sequence of SEQ ID 7 and light chain variable region comprising an amino acid sequence of SEQ ID 8. Methods of making and using GYM 329 are described in can be produced according to WO2016098357 and WO2017/104783. GYM 329 is known to be Fc engineered to enable remove antigen form plasma.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

The terms “anti-myostatin antibody” and “an antibody that binds to myostatin” refer to an antibody that is capable of binding myostatin with sufficient affinity such that the antibody is useful as a diagnostic and/or therapeutic agent in targeting myostatin. In one embodiment, the extent of binding of an anti-myostatin antibody to an unrelated, non-myostatin protein is less than about 10% of the binding of the antibody to myostatin as measured, e.g., by a radioimmunoassay (RIA). In certain embodiments, an antibody that binds to myostatin has a dissociation constant (Kd) of 1 micro M or less, 100 nM or less, 10 nM or less, 1 nM or less, 0.1 nM or less, 0.01 nM or less, or 0.001 nM or less (e.g., 10−8 M or less, e.g., from 10−8 M to 10−13 M, e.g., from 10−9 M to 10−13 M). In certain embodiments, an anti myostatin antibody binds to an epitope of myostatin that is conserved among myostatin from different species.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g., scFv); and multispecific antibodies formed from antibody fragments.

An “antibody that binds to the same epitope” as a reference antibody refers to an antibody that blocks binding of the reference antibody to its antigen in a competition assay, and/or conversely, the reference antibody blocks binding of the antibody to its antigen in a competition assay. An exemplary competition assay is provided herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called alpha, delta, epsilon, gamma, and mu, respectively.

The term “cytotoxic agent” as used herein refers to a substance that inhibits or prevents a cellular function and/or causes cell death or destruction. Cytotoxic agents include, but are not limited to, radioactive isotopes (e.g., At211, I131, I125, Y90, Re186, Re188, Sm153, Bi212, P32, Pb212 and radioactive isotopes of Lu); chemotherapeutic agents or drugs (e.g., methotrexate, adriamycin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed below.

The term “epitope” includes any determinant capable of being bound by an antibody. An epitope is a region of an antigen that is bound by an antibody that targets that antigen, and includes specific amino acids that directly contact the antibody. Epitope determinants can include chemically active surface groupings of molecules such as amino acids, sugar side chains, phosphoryl or sulfonyl groups, and can have specific three dimensional structural characteristics, and/or specific charge characteristics. Generally, antibodies specific for a particular target antigen will preferentially recognize an epitope on the target antigen in a complex mixture of proteins and/or macromolecules.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

The term “Fc region-comprising antibody” refers to an antibody that comprises an Fc region. The C-terminal lysine (residue 447 according to the EU numbering system) of the Fc region may be removed, for example, during purification of the antibody or by recombinant engineering of the nucleic acid encoding the antibody. Accordingly, a composition comprising an antibody having an Fc region according to this invention can comprise an antibody with K447, with all K447 removed, or a mixture of antibodies with and without the K447 residue.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The terms “full length antibody,” “intact antibody,” and “whole antibody” are used herein interchangeably to refer to an antibody having a structure substantially similar to a native antibody structure or having heavy chains that contain an Fc region as defined herein.

A “functional Fc region” possesses an “effector function” of a native sequence Fc region. Exemplary “effector functions” include C1q binding; CDC; Fc receptor binding; ADCC; phagocytosis; down regulation of cell surface receptors (e.g., B cell receptor; BCR), etc. Such effector functions generally require the Fc region to be combined with a binding domain (e.g., an antibody variable domain) and can be assessed using various assays as disclosed, for example, in definitions herein.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the US Copyright Office, Washington D.C., 20559, where it is registered under US Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary.

The term “myostatin”, as used herein, may refer to any native myostatin from any vertebrate source, including mammals such as primates (e.g., humans) and rodents (e.g., mice and rats). Unless otherwise indicated, the term “myostatin” refers to a human myostatin protein having the amino acid sequence shown in SEQ ID NO: 11 and containing the terminal propeptide domain of human myostatin as shown in SEQ ID NO: 12 or 13. The term encompasses “full-length”, unprocessed myostatin as well as any form of myostatin that results from processing in the cell. The term also encompasses naturally occurring variants of myostatin, e.g., splice variants or allelic variants. The amino acid sequence of an exemplary human myostatin (promyostatin) is shown in SEQ ID NO: 11. The amino acid sequence of an exemplary N-terminal propeptide domain of human myostatin is shown in SEQ ID NO: 12 or 13. Active mature myostatin is a disulfide-bonded homodimer consisting of two C-terminal growth factor domains. Inactive latent myostatin is a noncovalently-associated complex of two propeptides and the mature myostatin. As disclosed herein, the antibodies of the invention bind inactive latent myostatin, but do not bind the mature active myostatin homodimer. In some embodiments, the antibodies of the invention bind an epitope within a fragment consisting of amino acids 21-100 of myostatin propeptide (SEQ ID NO:13), but do not bind the mature active myostatin homodimer.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al., Kuby Immunology, 6th ed., W. H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

A “variant Fc region” comprises an amino acid sequence which differs from that of a native sequence Fc region by virtue of at least one amino acid modification (alteration), preferably one or more amino acid substitution(s). Preferably, the variant Fc region has at least one amino acid substitution compared to a native sequence Fc region or to the Fc region of a parent polypeptide, e.g., from about one to about ten amino acid substitutions, and preferably from about one to about five amino acid substitutions in a native sequence Fc region or in the Fc region of the parent polypeptide. The variant Fc region herein will preferably possess at least about 80% homology with a native sequence Fc region and/or with an Fc region of a parent polypeptide, and most preferably at least about 90% homology therewith, more preferably at least about 95% homology therewith.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”.

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise six HVRs: three in the VH (H1, H2, H3), and three in the VL (L1, L2, L3). Exemplary HVRs herein include: (a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987)); (b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, NIH, Bethesda, MD (1991)); (c) antigen contacts occurring at amino acid residues 27c-36 (L1), 46-55 (L2), 89-96 (L3), 30-35b (H1), 47-58 (H2), and 93-101 (H3) (MacCallum et al., J. Mol. Biol. 262: 732-745 (1996)); and (d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3). Unless otherwise indicated, HVR residues and other residues in the variable domain (e.g., FR residues) are numbered herein according to Kabat et al., supra.

The term “Revised Hammersmith Scale” also known as RHS acronym, is a psychometrically and clinically robust functional outcome measure designed specifically to assess physical abilities in strong ambulatory patients with Type 3 SMA through weak patients with Type 2 SMA. This scale was developed using the Hammersmith Functional Motor Scale Expanded (HFMSE) as a foundation; a measure widely used internationally in clinical practice, in clinical trials, and to document SMA natural history and trajectories of SMA disease course. One of the key strengths of the RHS is the robust development process, whereby an already well-developed and established scale was used as a foundation and expert panels were involved throughout to ensure clinical relevance of amendments. The psychometric analysis, facilitated by several international pilots, resulted in construction of a robust SMA-specific clinical outcome assessment tool (Ramsey et al. PLoS One 2017; 12:e0172346). The RHS consists of 36 items. To avoid the ceiling effect seen on other functional scales, the RHS contains revised items from the North Star Ambulatory Assessment (NSAA) including two timed tests (RHS item 19 [time to walk/run 10 m] and item 25 [time to rise from floor]). The addition of these items extends the range of the scale to assess strong ambulatory patients with SMA, such as this study's population, and the ordinal scoring used in both items has been shown to allow further discrimination between clinically different abilities (p<0.05; Ramsey et al. PLoS One 2017; 12:e0172346). The RHS has published evidence of content and construct validity and inter-rater reliability in SMA (Ramsey et al. Neuromuscular Disord 2015; 25:S195; PLoS One 2017; 12:e0172346).

The term “Motor Function Measure-32 item” (MFM32) refers to a valid and reliable clinician-reported assessment of motor function ability in neuromuscular diseases. The assessment is validated in individuals with neuromuscular diseases, including SMA, aged 2 years and older (Berard et al. Neuromuscul Disord 2005; 15:463-70; Trundell et al. Neurol Ther 2020; 9:575-584) and patients have confirmed it relates to activities of daily living (Duong et al. BMC Neurol 2021; 21:143). The MFM32 contains 32 items assessed in three domains of motor function: Domaine 1 (D1)(standing and transfers), Domain 2 (D2)(axial and proximal motor function), and Domain 3 (D3)(distal motor function). When considering each domain independently, each has a different capacity to detect change depending on the patient's ability. Vuillerot et al. (Arch Phys Med Rehabil 2013; 94:1555-61) reported that D2 demonstrates good responsiveness in patients with Type 2 SMA, and that D1 demonstrates good responsiveness in patients with Type 3 SMA. In an ambulant population such as that included in this study, it is hypothesized that these participants would have a greater capacity for improvement in D1 and D2, which contain items assessing their ability to stand and walk as well as their axial and proximal function. Conversely, as distal motor function remains relatively preserved until the later stages of the disease, D3 would have less sensitivity to change in these ambulant patients.

A phase II/III study is to evaluate the safety, tolerability, pharmacokinetics, pharmacodynamics and efficacy of GYM239, the humanized monoclonal antibody described herein, that binds to human latent myostatin, in combination with risdiplam (Evrysdi®) in ambulant pediatric patients (2-10 years of age) with spinal muscular atrophy (SMA). Although three treatments are already available for SMA patients, there continues to be an unmet medical need as patients treated with existing disease modifying therapies, may remain with significant motor deficiencies related to the skeletomuscular system.

The therapeutic approach to improve patient motor function is to directly target the skeletal muscle to reduce muscle atrophy and thus improve muscle strength in subjects having muscle conditions, such as SMA. Inhibition of myostatin (also known as Growth and Differentiation Factor 8, or GDF-8) offers a promising approach to increase muscle mass and function in patients having muscle conditions, such as SMA patients. Myostatin is a member of the TGF superfamily and a critical negative regulator of muscle growth. Genetic loss of myostatin results in significantly increased muscle mass, resulting from both muscle cell hypertrophy and hyperplasia (A. C. McPherron et al., Nature 387, 83-90, 1997). As with myostatin loss of function mutations, pharmacologic inhibition of myostatin also increases muscle mass, mediated via muscle hypertrophy but not hyperplasia (S. J. Lee et al. Proc Natl Acad Sci USA 98, 9306-9311, 2001).
The invention according to the present application described the surprising effect that the combination of treatment of a splicing modifier, such as risdiplam, with GYM329 has on the treatment of SMA. It is has been surprisingly found that GYM329 without an efficacious pretreatment with a splicing modifier, such as risdiplam has little impact on SMA.

The present invention relates to Risdiplam for use in the treatment, prevention, delaying progression and/or amelioration of SMA when used in combination with an antibody comprising a VH having at least 90% sequence identity to the amino acid sequence of SEQ ID NO: 7 and a VL having at least 90% sequence identity to the amino acid sequence of SEQ ID NO: 8.

In particular embodiment, the present invention relates to Risdiplam for use in the treatment, prevention, delaying progression and/or amelioration of SMA when used in combination with an antibody comprising a VH having sequence identity to the amino acid sequence of SEQ ID NO: 7 and a VL having sequence identity to the amino acid sequence of SEQ ID NO: 8.

In further embodiment, the present invention relates to Risdiplam for use in the treatment of SMA when used in combination with an antibody comprising a VH having sequence identity to the amino acid sequence of SEQ ID NO: 7 and a VL having sequence identity to the amino acid sequence of SEQ ID NO: 8.

In a particular embodiment, the antibody to be used in combination with risdiplam is anti myostatin antibody. More particularly, the antibody according to the invention comprises one or more CDR sequences, the variable heavy and light chain sequences or heavy chain and light chain sequences selected from those described in Table 1,

TABLE 1
Anti-Myostatin Antibody CDR sequences

| CDR | antibody GM329 | SEQ ID # |
|---|---|---|
| CDRH1 | HDDIS | SEQ ID 1 |
| CDRH2 | IISYAGSTYYASWAKG | SEQ ID 2 |
| CDRH3 | GVPAYSHGGDL | SEQ ID 3 |
| CDRL1 | TTSQSVYHENWLS | SEQ ID 4 |
| CDRL2 | WASTLAY | SEQ ID 5 |
| CDRL3 | AGGYGGGRYA | SEQ ID 6 |

TABLE 2
Anti-Myostatin Antibody sequence GYM329

| | Amino Acid Sequence | SEQ ID # |
|---|---|---|
| Heavy | QVQLVESGGGLVQPGGSLRLSCA | SEQ ID 7 |
| Chain | VSGIDLSHDDISWVRQAPGKGLE | |
| Variable | WVSIISYAGSTYYASWAKGRLTIS | |
| Region | KDTSKNQVVLTMTNMDPVDTAT | |
| | YYCARGVPAYSHGGDLWGQGTL | |
| | VTVSS | |
| Light | DIVMTQSPATLSLSPGERATLSCT | SEQ ID 8 |
| Chain | TSQSVYHENWLSWFQQKPGQPPK | |
| variable | LLIYWASTLAYGVPSRFSGSGSGT | |
| Region | DFTLTISSLQPEDAATYYCAGGYG | |
| | GGRYAFGQGTKVEIK | |
| Heavy | QVQLVESGGGLVQPGGSLRLSCA | SEQ ID 9 |
| Chain | VSGIDLSHDDISWVRQAPGKGLE | |
| | WVSIISYAGSTYYASWAKGRLTIS | |
| | KDTSKNQVVLTMTNMDPVDTAT | |
| | YYCARGVPAYSHGGDLWGQGTL | |
| | VTVSSASTKGPSVFPLAPSSKSTSG | |
| | GTAALGCLVKDYFPEPVTVSWNS | |
| | GALTSGVHTFPAVLQSSGLYSLSS | |
| | VVTVPSSSLGTQTYICNVNHKPSN | |
| | TKVDKKVEPKSCDKTHTCPPCPAP | |
| | EYLGGDSVFLFPPKPKDVLMISRT | |
| | PEVTCVVIDVSHEDPEVKFNWYV | |
| | DGVEVHNAKTKPREEQYNSTYRV | |
| | VSVLPVLHRDWLNGKEYKCKVS | |
| | NKALPKPIEKTISKAKGQRREPQV | |
| | YTLPPSREEMTKNQVSLTCLVKGF | |
| | YPSDIAVEWESNGQPENNYKTTPP | |
| | VLDSDGSFFLYSKLTVDKSRWQQ | |
| | GNVFSCSVLHEALHAHTTRKELSL | |
| | SP | |
| Light | DIVMTQSPATLSLSPGERATLSCT | SEQ 10 |
| Chain | TSQSVYHENWLSWFQQKPGQPPK | |
| | LLIYWASTLAYGVPSRFSGSGSGT | |
| | DFTLTISSLQPEDAATYYCAGGYG | |
| | GGRYAFGQGTKVEIKRTVAAPSV | |
| | FIFPPSDEQLKSGTASVVCLLNNF | |
| | YPREAKVQWKVDNALQSGNSQE | |
| | SVTEQDSKDSTYSLSSTLTLSKAD | |
| | YEKHKVYACEVTHQGLSSPVTKS | |
| | FNRGEC | |

In some embodiments, an isolated anti-myostatin antibody of the present invention is a monoclonal antibody. In some embodiments, an isolated anti-myostatin antibody of the present invention is a human, humanized, or chimeric antibody. In some embodiments, an isolated anti-myostatin antibody of the present invention is an antibody fragment that binds to myostatin. In some embodiments, an isolated anti-myostatin antibody of the present invention is an antibody fragment that binds to latent myostatin. In some embodiments, an isolated anti-myostatin antibody of the present invention is a full length IgG antibody.

An antibody or a polypeptide comprising a variant Fc region of the invention (and optionally any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antibodies or polypeptides comprising a variant Fc region of the invention can be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antibody of the invention will depend on the course of the disease and whether the antibody is administered for preventive or therapeutic purposes, previous therapy. The antibody or polypeptide comprising a variant Fc region of the invention is suitably administered to the patient at one time or over a series of treatments. Depending on the type and severity of the disease, about 1 micro g/kg to 15 mg/kg (e.g., 0.1 mg/kg-10 mg/kg) of antibody can be an initial candidate dosage for administration to the patient, whether, for example, by one or more separate administrations, or by continuous infusion. In particular the anti-myostatin may be administered intermittently, every week, every three weeks or particularly every four weeks, more particularly every four weeks. An initial higher loading dose, followed by one or more lower doses may be administered. The progress of this therapy is easily monitored by conventional techniques and assays.

According to the present invention, the anti myostatin maybe be formulated in a pharmaceutical formulation comprising the antibody and a pharmaceutically acceptable carrier.

Exemplary lyophilized antibody formulations are described in U.S. Pat. No. 6,267,958. Aqueous antibody formulations include those described in U.S. Pat. No. 6,171,586 and WO 2006/044908, the latter formulations including a histidine-acetate buffer.

In a further aspect, the invention provides pharmaceutical formulations comprising the anti-myostatin antibody provided herein, e.g., for use in SMA in combination with risdiplam. In one embodiment, a pharmaceutical formulation comprises the anti-myostatin antibody provided herein and a pharmaceutically acceptable carrier.

In a further aspect, the pharmaceutical formulation of the anti-myostatin as described herein is for treatment of SMA. The anti-myostatin antibody of the present invention may exhibit pH-dependent binding characteristics. In a further embodiment, the pharmaceutical formulation is for enhancing the clearance of myostatin from plasma. In one embodiment, the pharmaceutical formulation is administered to an individual having SMA.

The antibody or a polypeptide comprising a variant Fc region of the invention can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein. More particularly the administration according to the invention of the anti-myostatin antibody is to be administered every four weeks, more particularly via subcutaneous injections.

In a further aspect, the invention provides methods for preparing a medicament or a pharmaceutical formulation, comprising mixing the anti-myostatin antibody provided herein with a pharmaceutically acceptable carrier, e.g., for use in treating SMA.

The polypeptide comprising a variant Fc region provided herein may be used in therapeutic methods. In a further aspect, the invention provides pharmaceutical formulations comprising a polypeptide comprising any of the polypeptides comprising variant Fc regions provided herein, e.g., for use in treating SMA. In one embodiment, a pharmaceutical formulation comprises a polypeptide comprising any of the polypeptides comprising variant Fc regions provided herein and a pharmaceutically acceptable carrier.

Muscle atrophy is a significant clinical hallmark of disease progression in SMA. In those patients with more severe disease, such progression leads to a reduction in functional muscle of the upper and lower limbs (Chabanon et al. PLoS One 2018; 13:e0201004). Data from patients with neuromuscular diseases have indicated that circulating concentrations of myostatin are decreased with disease progression (Burch et al, J Neurol 2017; 264:541-553). Considering that myostatin is the target of GYM329, the ambulant SMA sub-population was considered to have the greatest potential to demonstrate the benefit of an anti-myostatin therapy in SMA in a clinical study setting due to greater functional muscle preservation as a result of a less progressed disease.

To avoid confounding results with the physical changes occurring during puberty, patients older than 10 years were not included in this specific clinical trial. This does not imply necessarily that the combination treatment would be limited to patients younger than 10 years old. The treatment should be available to older SMA patients.

According to the invention, an effective amount of the myostatin inhibitor to treat a muscle condition is an amount that achieves both clinical efficacy and safety. In some embodiments, the effective amount is an amount that enhances muscle function, such as force generation and motor function. In some embodiments, the effective amount is an amount that enhances motor function that requires fast-twitch fibers (e.g., type II fibers). In some embodiments, the motor function comprises eccentric contraction of a muscle. In some embodiments, an effective amount of the myostatin therapy is an amount sufficient to delay or alleviate progression of disease (e.g., muscle atrophy); maintain disease status (e.g., as measured/monitored by a suitable motor function test, plasma protein markers, metabolic markers, etc.); delay loss of a-motor neurons; prevent or delay expression of immature muscle markers; prevent, alleviate or delay intramuscular fat deposits (e.g., fatty replacement of muscle tissue); prevent metabolic dysregulation; prevent or reduce bone loss or frequency of bone fracture; increase an Expanded Hammersmith Functional Motor Scale score by >1 point 1 as compared to control that does not receive the myostatin inhibitor; slow the rate of deterioration; delay regression (e.g., progressive decrease) of an Expanded Hammersmith Functional Motor Scale over a period of 12 months, 24 months or 36 months; and/or, increase a CHOP INTEND score by >1 point as compared to control that does not receive the myostatin inhibitor; and/or, increase a MFM-32 score by >1 point as compared to control that does not receive the myostatin inhibitor.

In some embodiments, the muscle condition to be treated with a myostatin inhibitor is associated with a neuromuscular disease, including but are not limited to: Amyotrophic lateral sclerosis (ALS); Congenital myasthenic syndrome; Congenital myopathy; Cramp fasciculation syndrome; Duchenne muscular dystrophy (DMD); Glycogen storage disease type II; Hereditary spastic paraplegia; Inclusion body myositis (IBM); Isaac's Syndrome; Kearns-Sayre syndrome; Lambert-Eaton myasthenic syndrome; Mitochondrial myopathy; Muscular dystrophy; Myasthenia gravis; Myotonic dystrophy; Peripheral neuropathy; Spinal and bulbar muscular atrophy; Spinal muscular atrophy (SMA); Spinal muscular atrophy with respiratory distress type 1; Stiff person syndrome; Troyer syndrome; and, Guillain-Barre syndrome.

According to the here within described invention more particular embodiments of the invention are described below:

Embodiment 1. Risdiplam for use in the treatment, prevention, delaying progression and/or amelioration of SMA, in particular in a patient, when used in combination with an anti-myostatin antibody comprising six complementary determining regions (CDRs): CDRH1, CDRH2, CDRH3, CDRL1, CDRL2 and CDRL3 wherein CDRH1 comprises a sequence set forth in SEQ ID 1, CDRH2 comprises a sequence set forth in SEQ ID 2, CDRH3 comprises a sequence set forth in SEQ ID 3, CDRL1 comprises a sequence set forth in SEQ ID 4, CDRL2 comprises a sequence set forth in SEQ ID 5 and CDRL3 comprises a sequence set forth in SEQ ID 6.

Embodiment 2. Risdiplam for use in the treatment of SMA in combination with an anti-myostatin antibody comprising six complementary determining regions (CDRs): CDRH1, CDRH2, CDRH3, CDRL1, CDRL2 and CDRL3 wherein CDRH1 comprises a sequence set forth in SEQ ID 1, CDRH2 comprises a sequence set forth in SEQ ID 2, CDRH3 comprises a sequence set forth in SEQ ID 3, CDRL1 comprises a sequence set forth in SEQ ID 4, CDRL2 comprises a sequence set forth in SEQ ID 5 and CDRL3 comprises a sequence set forth in SEQ ID 6.

Embodiment 3. The risdiplam for use in the treatment of SMA of embodiment 1 or 2, wherein the anti-myostatin antibody inhibits activation of myostatin.

Embodiment 4. The risdiplam for use in the treatment SMA according to any one of embodiments 1 to 3, wherein the anti-myostatin antibody blocks the proteolytic release of mature myostatin.

Embodiment 5. Risdiplam for use in the treatment according to any one of embodiments 1 to 4, wherein the anti-myostatin antibody comprises comprising a VH having at least 90% sequence identity to the amino acid sequence of SEQ ID 7 and a VL having at least 90% sequence identity to the amino acid sequence of SEQ ID 8.

Embodiment 6. Risdiplam for use in the treatment according to any one of 1 to 5, wherein the anti-myostatin antibody comprises a heavy chain variable region comprising an amino acid sequence of SEQ ID 7 and light chain variable region comprising an amino acid sequence of SEQ ID 8.

Embodiment 7. Risdiplam for use in the treatment according to any one of embodiments 1 to 6, wherein the anti-myostatin antibody comprises a heavy chain region comprising an amino acid sequence of SEQ ID 9 and light chain region comprising an amino acid sequence of SEQ ID 10.

Embodiment 8. Risdiplam for use in the treatment according to any one of embodiments 1 to 7, wherein the anti-myostatin antibody is GM329.

Embodiment 9. Risdiplam for use in the treatment according to any one of embodiments 1 to 8, in patient (in particular a patient in need thereof), particularly wherein the patient is a human (such as a male or female human).

Embodiment 10. Risdiplam for use in the treatment according to any one of embodiments 1 to 9, wherein the patient to be treated has been first treated with risdiplam for at least 2 weeks, particularly for at least 3 weeks, more particularly for at least 4 weeks, even more particularly for at least 6 weeks, most particularly for at least 8 weeks, before the antibody first dose is being administered to the patient.

Embodiment 11. Risdiplam for use in the treatment according to any one of embodiments 1 to 10, wherein the total daily dose of risdiplam is administered to the patient at 0.2 mg/kg for patients between 2 months and 2 years, at 0.25 mg/kg for patients older than 2 years and with a body weight of less than 20 kg, and at 5 mg for patients with a body weight of more than or equal to 20 kg.

Embodiment 12. Risdiplam for use in the treatment according to any one of embodiments 1 to 11, wherein the anti-myostatin antibody dose is administered to the patient at 7.4 mg or 24 mg for patients older than 2 years and with a body weight of less than 20 kg, and at 10.6 mg or 36 mg for patients with a body weight of more than or equal to 20 kg, particularly wherein the antibody dose is administered every four weeks to the patient at 24 mg for patients older than 2 years and with a body weight of less than 20 kg, and at 36 mg for patients with a body weight of more than or equal to 20 kg.

Embodiment 13. Risdiplam for use in the treatment according to any one of embodiments 1 to 12, wherein the anti-myostatin antibody is being administered every four weeks.

Embodiment 14. Risdiplam for use in the treatment according to any one of embodiments 1 to 13, wherein the patient has SMA.

Embodiment 15. Risdiplam for use in the treatment according to any one of embodiments 1 to 14, wherein SMA is type I SMA; type II SMA or type III SMA.

Embodiment 16. The risdiplam for use in treating SMA according to any one of embodiments 1 to 15, wherein the patient to be treated has been first treated with risdiplam for at least 2 weeks, particularly for at least 3 weeks, more particularly for at least 4 weeks, even more particularly for at least 6 weeks, most particularly for at least 8 weeks, before anti-myostatin is being first administered.

Embodiment 17. Risdiplam for use in the treatment according to any one of embodiments 1 to 16, wherein risdiplam is being administered in a pharmaceutical composition comprising:
1 to 10% wt of risdiplam or a pharmaceutically acceptable salt thereof;
2 to 15% wt, in particular 4 to 6% wt of a buffer system, particularly a buffer system selected from citrate, malate, maleate, or tartrate, more particularly malate or tartrate, most particularly tartrate; or alternatively the
corresponding acid of a buffer system alone as acidifier, particularly tartaric acid;
40 to 90% wt of a diluent, particularly mannitol or a mixture of mannitol and isomalt, more particularly mannitol;
0.5 to 4% wt of an antioxidant, particularly ascorbic acid;
0.2 to 2% wt of a stabilizer, particularly disodium edetate;
0.5 to 2% wt of a lubricant, particularly PEG6000;
1 to 8% wt, in particular 1 to 4% wt, of a preservative selected from potassium sorbate or sodium benzoate;
0 to 3% wt of a sweetener, particularly sucralose or sodium saccharin, most particularly sucralose; and
0 to 20% wt of a flavor, particularly strawberry flavor or vanilla flavor; wherein the total amount of ingredients does not exceed 100% wt.

Embodiment 18. Risdiplam for use in the treatment according to any one of embodiments 1 to 17, wherein risdiplam is being administered in the pharmaceutical composition comprising:
1 to 5% wt of risdiplam or a pharmaceutically acceptable salt thereof;
2 to 8% wt, in particular 4 to 6% wt of a tartrate buffer system;
60 to 75% wt of a mannitol as first diluent and 10 to 15% wt of isomalt as second diluent;
0.5 to 1.5% wt of ascorbic acid as antioxidant;
0.25 to 0.75% wt of disodium edetate as stabilizer;
0.5 to 2% wt of PEG6000 as lubricant;
1 to 8% wt, in particular 1 to 4% wt, of sodium benzoate as a preservative;
0.5 to 1% wt of sucralose as sweetener; and
5 to 10% wt of strawberry flavor;
wherein the total amount of ingredients does not exceed 100% wt.
A combination of a risdiplam and an anti-myostatin antibody comprising a six complementary determining regions (CDRs): CDRH1, CDRH2, CDRH3, CDRL1, CDRL2 and CDRL3 wherein CDRH1 comprises a sequence set forth in SEQ ID 1, CDRH2 comprises a sequence set forth in SEQ ID 2, CDRH3 comprises a sequence set forth in SEQ ID 3, CDRL1 comprises a sequence set forth in SEQ ID 4, CDRL2 comprises a sequence set forth in SEQ ID 5 and CDRL3 comprises a sequence set forth in SEQ ID 6.

Embodiment 19. The combination according to claim 19, wherein the anti-myostatin antibody comprises a VH having at least 90% sequence identity to the amino acid sequence of SEQ ID 7 and a VL having at least 90% sequence identity to the amino acid sequence of SEQ ID 8, for use in the treatment, prevention, delaying progression and/or amelioration of SMA.

Embodiment 20. The combination according to claim 19 or 20, wherein the anti-myostatin antibody comprises a heavy chain variable region comprising an amino acid sequence of SEQ ID 7 and light chain variable region comprising an amino acid sequence of SEQ ID 8.

Embodiment 21. The combination according to any one of embodiments 19 to 21, wherein the anti-myostatin antibody comprises a heavy chain comprising an amino acid sequence of SEQ ID 9 and light chain comprising an amino acid sequence of SEQ ID 10.

Embodiment 22. The combination according to any one of embodiments 19 to 21, in patient (in particular a patient in need thereof), particularly wherein the patient is a human (such as a male or female human).

Embodiment 23. The combination according to any one of embodiments 19 to 23, wherein the patient to be treated has been first treated with risdiplam for at least 2 weeks, particularly for at least 3 weeks, more particularly for at least 4 weeks, even more particularly for at least 6 weeks, most particularly for at least 8 weeks, before the antibody first dose is being administered to the patient.

Embodiment 24. The combination according to any one of embodiments 19 to 24, wherein the total daily dose of risdiplam is administered to the patient at 0.2 mg/kg for patients between 2 months and 2 years, at 0.25 mg/kg for patients older than 2 years and with a body weight of less than 20 kg, and at 5 mg for patients with a body weight of more than or equal to 20 kg.

Embodiment 25. The combination according to any one of embodiments 19 to 25, wherein the anti-myostatin antibody dose is administered to the patient at 7.4 mg or 24 mg for patients older than 2 years and with a body weight of less than 20 kg, and at 10.6 mg or 36 mg for patients with a body weight of more than or equal to 20 kg, particularly wherein the antibody dose is administered every four weeks to the patient at 24 mg for patients older than 2 years and with a body weight of less than 20 kg, and at 36 mg for patients with a body weight of more than or equal to 20 kg.

Embodiment 26. The combination according to any one of embodiments 19 to 26, wherein the anti-myostatin antibody is GM329.

Embodiment 27. The combination according to any one of embodiments 19 to 27, wherein the anti-myostatin antibody is being administered every four weeks.

Embodiment 28. The combination according to any one of embodiments 19 to 28, wherein the patient has SMA.

Embodiment 29. The combination according to any one of embodiments 19 to 29, wherein SMA is type I SMA; type II SMA or type III SMA.

Embodiment 30. The combination according to any one of embodiments 19-21 or 23-30, wherein risdiplam is being administered in a pharmaceutical composition comprising:
1 to 10% wt of risdiplam or a pharmaceutically acceptable salt thereof;
2 to 15% wt, in particular 4 to 6% wt of a buffer system, particularly a buffer system selected from citrate, malate, maleate, or tartrate, more particularly malate or tartrate, most particularly tartrate; or alternatively the corresponding acid of a buffer system alone as acidifier, particularly tartaric acid;
40 to 90% wt of a diluent, particularly mannitol or a mixture of mannitol and isomalt, more particularly mannitol;
0.5 to 4% wt of an antioxidant, particularly ascorbic acid;
0.2 to 2% wt of a stabilizer, particularly disodium edetate;
0.5 to 2% wt of a lubricant, particularly PEG6000;
1 to 8% wt, in particular 1 to 4% wt, of a preservative selected from potassium sorbate or sodium benzoate;
0 to 3% wt of a sweetener, particularly sucralose or sodium saccharin, most particularly sucralose; and
0 to 20% wt of a flavor, particularly strawberry flavor or vanilla flavor; wherein the total amount of ingredients does not exceed 100% wt.

Embodiment 31. The combination according to any one of embodiments 19-21 or 23-31, wherein risdiplam is being administered in the pharmaceutical composition comprising:
1 to 5% wt of risdiplam or a pharmaceutically acceptable salt thereof;
2 to 8% wt, in particular 4 to 6% wt of a tartrate buffer system;
60 to 75% wt of a mannitol as first diluent and 10 to 15% wt of isomalt as second diluent;
0.5 to 1.5% wt of ascorbic acid as antioxidant;
0.25 to 0.75% wt of disodium edetate as stabilizer;
0.5 to 2% wt of PEG6000 as lubricant;
1 to 8% wt, in particular 1 to 4% wt, of sodium benzoate as a preservative;
0.5 to 1% wt of sucralose as sweetener; and
5 to 10% wt of strawberry flavor;
wherein the total amount of ingredients does not exceed 100% wt.

Embodiment 32. A method for the treatment, prevention, delaying progression and/or amelioration of SMA in a subject in need thereof, wherein the patient is being treated
a) with risdiplam for at least 2 weeks, particularly for at least 3 weeks, more particularly for at least 4 weeks, even more particularly for at least 6 weeks, most particularly for at least 8 weeks, then
b) with risdiplam and an anti-myostatin antibody comprising a six complementary determining regions (CDRs): CDRH1, CDRH2, CDRH3, CDRL1, CDRL2 and CDRL3 wherein CDRH1 comprises a sequence set forth in SEQ ID 1, CDRH2 comprises a sequence set forth in SEQ ID 2, CDRH3 comprises a sequence set forth in SEQ ID 3, CDRL1 comprises a sequence set forth in SEQ ID 4, CDRL2 comprises a sequence set forth in SEQ ID 5 and CDRL3 comprises a sequence set forth in SEQ ID 6.

Embodiment 33. A method for the treatment, prevention, delaying progression and/or amelioration of SMA which method comprises administering a combination of risdiplam and an anti-myostatin antibody comprising a six complementary determining regions (CDRs): CDRH1, CDRH2, CDRH3, CDRL1, CDRL2 and CDRL3 wherein CDRH1 comprises a sequence set forth in SEQ ID 1, CDRH2 comprises a sequence set forth in SEQ ID 2, CDRH3 comprises a sequence set forth in SEQ ID 3, CDRL1 comprises a sequence set forth in SEQ ID 4, CDRL2 comprises a sequence set forth in SEQ ID 5 and CDRL3 comprises a sequence set forth in SEQ ID 6.

Embodiment 34. The method of embodiment 33 or 34, wherein the anti-myostatin antibody comprises a VH having at least 90% sequence identity to the amino acid sequence of SEQ ID 7 and a VL having at least 90% sequence identity to the amino acid sequence of SEQ ID 8.

Embodiment 35. The method of any one of embodiments 33 to 35, wherein the anti-myostatin antibody comprises a heavy chain comprising an amino acid sequence of SEQ ID 9 and light chain comprising an amino acid sequence of SEQ ID 10.

Embodiment 36. The method of any one of embodiments 33 to 36, wherein the total daily dose of risdiplam is administered to the patient at 0.2 mg/kg for patients between 2 months and 2 years, at 0.25 mg/kg for patients older than 2 years and with a body weight of less than 20 kg, and at 5 mg for patients with a body weight of more than or equal to 20 kg.

Embodiment 37. The method of any one of embodiments 33 to 37, wherein the antibody dose is administered every four weeks to the patient at 7.4 mg or 24 mg for patients older than 2 years and with a body weight of less than 20 kg, and at 10.6 mg or 36 mg for patients with a body weight of more than or equal to 20 kg, particularly wherein the antibody dose is administered every four weeks to the patient at 24 mg for patients older than 2 years and with a body weight of less than 20 kg, and at 36 mg for patients with a body weight of more than or equal to 20 kg.

Embodiment 38. The method of any one of embodiments 33 to 38, wherein the patient has a type I SMA; type II SMA or type III SMA.

Embodiment 39. The method of any one of embodiments 33 to 39, in patient (in particular a patient in need thereof), particularly wherein the patient is a human (such as a male or female human).

Embodiment 40. The method of any one of embodiments 34 to 40, wherein the patient to be treated has been first treated with risdiplam for at least 2 weeks, particularly for at least 3 weeks, more particularly for at least 4 weeks, even more particularly for at least 6 weeks, most particularly for at least 8 weeks, before the antibody first dose is being administered to the patient.

Embodiment 41. The method of any one of embodiments 33 to 41, wherein the total daily dose of risdiplam is administered to the patient at 0.2 mg/kg for patients between 2 months and 2 years, at 0.25 mg/kg for patients older than 2 years and with a body weight of less than 20 kg, and at 5 mg for patients with a body weight of more than or equal to 20 kg.

Embodiment 42. The method of any one of embodiments 33 to 42, wherein the anti-myostatin antibody dose is administered to the patient at 7.4 mg or 24 mg for patients older than 2 years and with a body weight of less than 20 kg, and at 10.6 mg or 36 mg for patients with a body weight of more than or equal to 20 kg, particularly wherein the antibody dose is administered every four weeks to the patient at 24 mg for patients older than 2 years and with a body weight of less than 20 kg, and at 36 mg for patients with a body weight of more than or equal to 20 kg.

Embodiment 43. The method of any one of embodiments 33 to 43, wherein the anti-myostatin antibody is GM329.

Embodiment 44. The method of any one of embodiments 33 to 44, wherein the anti-myostatin antibody is being administered every four weeks.

Embodiment 45. The method of any one of embodiments 33 to 45, wherein the patient has SMA.

Embodiment 46. The method of any one of embodiments 33 to 46, wherein SMA is type I SMA; type II SMA or type III SMA.

Embodiment 47. The method of any one of embodiments 33 to 47, wherein risdiplam is being administered in a pharmaceutical composition comprising:
1 to 10% wt of risdiplam or a pharmaceutically acceptable salt thereof;
2 to 15% wt, in particular 4 to 6% wt of a buffer system, particularly a buffer system selected from citrate, malate, maleate, or tartrate, more particularly malate or tartrate, most particularly tartrate; or alternatively the corresponding acid of a buffer system alone as acidifier, particularly tartaric acid;
40 to 90% wt of a diluent, particularly mannitol or a mixture of mannitol and isomalt, more particularly mannitol;
0.5 to 4% wt of an antioxidant, particularly ascorbic acid;
0.2 to 2% wt of a stabilizer, particularly disodium edetate;
0.5 to 2% wt of a lubricant, particularly PEG6000;
1 to 8% wt, in particular 1 to 4% wt, of a preservative selected from potassium sorbate or sodium benzoate;
0 to 3% wt of a sweetener, particularly sucralose or sodium saccharin, most particularly sucralose; and
0 to 20% wt of a flavor, particularly strawberry flavor or vanilla flavor; wherein the total amount of ingredients does not exceed 100% wt.

Embodiment 48. The method of any one of embodiments 33 to 48, wherein risdiplam is being administered in a pharmaceutical composition comprises:
1 to 5% wt of risdiplam or a pharmaceutically acceptable salt thereof;
2 to 8% wt, in particular 4 to 6% wt of a tartrate buffer system;
60 to 75% wt of a mannitol as first diluent and 10 to 15% wt of isomalt as second diluent;
0.5 to 1.5% wt of ascorbic acid as antioxidant;
0.25 to 0.75% wt of disodium edetate as stabilizer;
0.5 to 2% wt of PEG6000 as lubricant;
1 to 8% wt, in particular 1 to 4% wt, of sodium benzoate as a preservative;
0.5 to 1% wt of sucralose as sweetener; and
5 to 10% wt of strawberry flavor;
wherein the total amount of ingredients does not exceed 100% wt.

Embodiment 49. A use of risdiplam in the manufacture of a medicament for the treatment of SMA, wherein the subjects treated with risdiplam are in addition treated with an anti-myostatin antibody comprising an anti-myostatin antibody comprising a six complementary determining regions (CDRs): CDRH1, CDRH2, CDRH3, CDRL1, CDRL2 and CDRL3 wherein CDRH1 comprises a sequence set forth in SEQ ID 1, CDRH2 comprises a sequence set forth in SEQ ID 2, CDRH3 comprises a sequence set forth in SEQ ID 3, CDRL1 comprises a sequence set forth in SEQ ID 4, CDRL2 comprises a sequence set forth in SEQ ID 5 and CDRL3 comprises a sequence set forth in SEQ ID 6.

Embodiment 50. The use of embodiment 50, wherein the anti-myostatin antibody comprises a VH having at least 90% sequence identity to the amino acid sequence of SEQ ID 7 and a VL having at least 90% sequence identity to the amino acid sequence of SEQ ID 8.

Embodiment 51. The use of embodiment 50, wherein the anti-myostatin antibody comprises a heavy chain comprising an amino acid sequence of SEQ ID 9 and light chain comprising an amino acid sequence of SEQ ID 10.

Embodiment 52. Risdiplam and GYM329 for use in the treatment, prevention, delaying progression and/or amelioration of SMA.

Embodiment 53. Risdiplam and GYM329 for use in the treatment, prevention, delaying progression and/or amelioration of SMA in a patient.

Embodiment 54. Risdiplam and GYM329 for use according to embodiment 53 or 54, wherein the patient to be treated is already been treated with risdiplam.

Embodiment 55. Risdiplam and GYM329 for use according to any one of embodiments 53 to 55, wherein the patient to be treated has been first treated with risdiplam for at least 2 weeks, particularly for at least 3 weeks, more particularly for at least 4 weeks, even more particularly for at least 6 weeks, most particularly for at least 8 weeks, before GYM329 is being first administered with risdiplam.

Embodiment 56. Risdiplam for use in the treatment, prevention, delaying progression and/or amelioration of SMA in a patient when used in combination with an antibody comprising the antibody comprises six complementary determining regions (CDRs): CDRH1, CDRH2, CDRH3, CDRL1, CDRL2 and CDRL3 wherein CDRH1 comprises a sequence set forth in SEQ ID 1, CDRH2 comprises a sequence set forth in SEQ ID 2, CDRH3 comprises a sequence set forth in SEQ ID 3, CDRL1 comprises a sequence set forth in SEQ ID 4, CDRL2 comprises a sequence set forth in SEQ ID 5 and CDRL3 comprises a sequence set forth in SEQ ID 6.

Embodiment 57. Risdiplam for use in the treatment according to embodiment 57, wherein a VH having at least 90% sequence identity to the amino acid sequence of SEQ ID 7 and a VL having at least 90% sequence identity to the amino acid sequence of SEQ ID 8.

Embodiment 58. Risdiplam for use in the treatment according to embodiment 57 or 58, wherein the antibody comprises a heavy chain variable region comprising an amino acid sequence of SEQ ID 7 and light chain variable region comprising an amino acid sequence of SEQ ID 8.

Embodiment 59. Risdiplam for use in the treatment according to any one of embodiments 57 to 59, wherein the anti-myostatin antibody comprises a heavy chain comprising an amino acid sequence of SEQ ID 9 and light chain comprising an amino acid sequence of SEQ ID 10.

Embodiment 60. Risdiplam for use in the treatment according to any one of embodiments 57 to 60, wherein the patient has been treated.

Embodiment 61. Use of risdiplam and GYM329 in treatment of SMA of a patient.

Embodiment 62. The use of claim 62, wherein the patient is a human (such as a male or female human).

Embodiment 63. The use of claim 62 or 63, wherein SMA is type I SMA; type II SMA or type III SMA.

Embodiment 64. The use according to any one of embodiments 62 to 64, wherein the total daily dose of risdiplam is administered to the patient at 0.2 mg/kg for patients between 2 months and 2 years, at 0.25 mg/kg for patients older than 2 years and with a body weight of less than 20 kg, and at 5 mg for patients with a body weight of more than or equal to 20 kg.

Embodiment 65. The use according to any one of embodiments 62 to 65, wherein the antibody dose is administered every four weeks to the patient at 7.4 mg or 24 mg for patients older than 2 years and with a body weight of less than 20 kg, and at 10.6 mg or 36 mg for patients with a body weight of more than or equal to 20 kg, particularly wherein the antibody dose is administered every four weeks to the patient at 24 mg for patients older than 2 years and with a body weight of less than 20 kg, and at 36 mg for patients with a body weight of more than or equal to 20 kg.

Embodiment 66. A package or kit comprising (a) risdiplam, optionally in a container, and (b) a package insert, package label, instructions or other labeling for the use according to any one of embodiments 62 to 66.

Embodiment 67. A package or kit according to claim 67 which further comprise (c) GYM329.

Embodiment 68. According to any of the embodiment mentioned herein, the patient to be treated start their treatment (in particular the risdiplam+anti-myostatin antibody) between the ages of 2 to 10 years old.

Embodiment 69. An anti-myostatin antibody for use in the treatment, prevention, delaying progression and/or amelioration of SMA when used in combination with Risdiplam, in particular in a patient, wherein the myostatin antibody comprises six complementary determining regions (CDRs): CDRH1, CDRH2, CDRH3, CDRL1, CDRL2 and CDRL3 wherein CDRH1 comprises a sequence set forth in SEQ ID 1, CDRH2 comprises a sequence set forth in SEQ ID 2, CDRH3 comprises a sequence set forth in SEQ ID 3, CDRL1 comprises a sequence set forth in SEQ ID 4, CDRL2 comprises a sequence set forth in SEQ ID 5 and CDRL3 comprises a sequence set forth in SEQ ID 6.

Embodiment 70. An anti-myostatin antibody for use in the treatment of SMA in combination with risdiplam wherein the anti-myostatin comprises six complementary determining regions (CDRs): CDRH1, CDRH2, CDRH3, CDRL1, CDRL2 and CDRL3 wherein CDRH1 comprises a sequence set forth in SEQ ID 1, CDRH2 comprises a sequence set forth in SEQ ID 2, CDRH3 comprises a sequence set forth in SEQ ID 3, CDRL1 comprises a sequence set forth in SEQ ID 4, CDRL2 comprises a sequence set forth in SEQ ID 5 and CDRL3 comprises a sequence set forth in SEQ ID 6.

Embodiment 71. The anti-myostatin antibody use in the treatment of SMA of Embodiment 69 or Embodiment 70, wherein the anti-myostatin antibody inhibits activation of myostatin.

Embodiment 72. The anti-myostatin antibody for use in the treatment SMA according to any one of embodiments 69 to 71, wherein the anti-myostatin antibody blocks the proteolytic release of mature myostatin.

Embodiment 73. The anti-myostatin antibody for use in the treatment according to any one of embodiments 69 to 72, wherein the anti-myostatin antibody comprises comprising a VH having at least 90% sequence identity to the amino acid sequence of SEQ ID 7 and a VL having at least 90% sequence identity to the amino acid sequence of SEQ ID 8.

Embodiment 74. The anti-myostatin antibody for use in the treatment according to any one of embodiments 69 to 73, wherein the anti-myostatin antibody comprises a heavy chain variable region comprising an amino acid sequence of SEQ ID 7 and light chain variable region comprising an amino acid sequence of SEQ ID 8.

Embodiment 75. The anti-myostatin antibody for use in the treatment according to any one of embodiments 69 to 74, wherein the anti-myostatin antibody comprises a heavy chain region comprising an amino acid sequence of SEQ ID 9 and light chain region comprising an amino acid sequence of SEQ ID 10.

Embodiment 76. The anti-myostatin antibody for use in the treatment according to any one of embodiments 69 to 75, wherein the anti-myostatin antibody is GM329.

Embodiment 77. The anti-myostatin antibody for use in the treatment according to any one of embodiments 69 to 76, in patient (in particular a patient in need thereof), particularly wherein the patient is a human (such as a male or female human).

Embodiment 78. The anti-myostatin antibody for use in the treatment according to any one of embodiments 69 to 77, wherein the patient to be treated has been first treated with risdiplam for at least 2 weeks, particularly for at least 3 weeks, more particularly for at least 4 weeks, even more particularly for at least 6 weeks, most particularly for at least 8 weeks, before the antibody first dose is being administered to the patient.

Embodiment 79. The anti-myostatin antibody for use in the treatment according to any one of embodiments 69 to 78, wherein the total daily dose of risdiplam is administered to the patient at 0.2 mg/kg for patients between 2 months and 2 years, at 0.25 mg/kg for patients older than 2 years and with a body weight of less than 20 kg, and at 5 mg for patients with a body weight of more than or equal to 20 kg.

Embodiment 80. The anti-myostatin antibody for use in the treatment according to any one of embodiments 69 to 79, wherein the anti-myostatin antibody dose is administered to the patient at 7.4 mg or 24 mg for patients older than 2 years and with a body weight of less than 20 kg, and at 10.6 mg or 36 mg for patients with a body weight of more than or equal to 20 kg, particularly wherein the antibody dose is administered every four weeks to the patient at 24 mg for patients older than 2 years and with a body weight of less than 20 kg, and at 36 mg for patients with a body weight of more than or equal to 20 kg.

Embodiment 81. The anti-myostatin antibody for use in the treatment according to any one of embodiments 69 to 80, wherein the anti-myostatin antibody is being administered every four weeks.

Embodiment 82. The anti-myostatin antibody for use in the treatment according to any one of embodiments 69 to 81, wherein the patient has SMA.

Embodiment 83. The anti-myostatin antibody for use in the treatment according to any one of embodiments 69 to 82, wherein SMA is type I SMA; type II SMA or type III SMA.

Embodiment 84. The anti-myostatin antibody for use in the treatment according to any one of embodiments 69 to 83, wherein risdiplam is being administered in a pharmaceutical composition comprising:
1 to 10% wt of risdiplam or a pharmaceutically acceptable salt thereof;
2 to 15% wt, in particular 4 to 6% wt of a buffer system, particularly a buffer system selected from citrate, malate, maleate, or tartrate, more particularly malate or tartrate, most particularly tartrate; or alternatively the corresponding acid of a buffer system alone as acidifier, particularly tartaric acid;
40 to 90% wt of a diluent, particularly mannitol or a mixture of mannitol and isomalt, more particularly mannitol;
0.5 to 4% wt of an antioxidant, particularly ascorbic acid;
0.2 to 2% wt of a stabilizer, particularly disodium edetate;
0.5 to 2% wt of a lubricant, particularly PEG6000;
1 to 8% wt, in particular 1 to 4% wt, of a preservative selected from potassium sorbate or sodium benzoate;
0 to 3% wt of a sweetener, particularly sucralose or sodium saccharin, most particularly sucralose; and
0 to 20% wt of a flavor, particularly strawberry flavor or vanilla flavor; wherein the total amount of ingredients does not exceed 100% wt.

Embodiment 85. The anti-myostatin antibody for use in the treatment according to any one of embodiments 69 to 84, wherein risdiplam is being administered in the pharmaceutical composition comprising:
1 to 5% wt of risdiplam or a pharmaceutically acceptable salt thereof;
2 to 8% wt, in particular 4 to 6% wt of a tartrate buffer system;
60 to 75% wt of a mannitol as first diluent and 10 to 15% wt of isomalt as second diluent;
0.5 to 1.5% wt of ascorbic acid as antioxidant;
0.25 to 0.75% wt of disodium edetate as stabilizer;
0.5 to 2% wt of PEG6000 as lubricant;
1 to 8% wt, in particular 1 to 4% wt, of sodium benzoate as a preservative;
0.5 to 1% wt of sucralose as sweetener; and
5 to 10% wt of strawberry flavor;
wherein the total amount of ingredients does not exceed 100% wt.
   The following examples are intended merely to illustrate the practice of the present invention and is not provided by way of limitation.

The following abbreviations and definitions are used: ADA (anti-drug antibody), ASO (antisense oligonucleotides), AUC (area under the concentration-time curve), BW (body weight), CaGI-C (caregiver global impression of change), Cohort (CH), Cmax (maximum concentration), CPK-MB (creatine phosphkinase kinase myocardial band), CRF (Case Report Form), CRS (cytokine release syndrome), CSA (cross-sectional area), cTnI (cardiac troponin I), cTnT (cardiac troponin T), D1 (Domain 1), D2 (Domain 2), D3 (Domain 3), DXA (dual-energy X-ray absorptiometry), EC (Ethics Committee), eCOA (electronic clinical outcome assessment), eCRF (electronic Case Report Form), EDC (electronic data capture), EIH (entry-into-human), EQ-5D-5L (EuroQoL 5 Dimension-5 Level), GDF-8 (growth differentiation factor-8), HDAC (histone deacetylase), HFMSE (Hammersmith Functional Motor Scale Expanded), ICH (International Council for Harmonisation), iDCC (independent Data Coordinating Center), iDMC (independent Data Monitoring Committee), IMC (Internal Monitoring Committee), IMP (investigational medicinal product), IRB (Institutional Review Board), ITT (intent-to-treat), IxRS (interactive voice or web-based response system), mAb (monoclonal antibody), MAR (missing at random), MATE (multidrug and toxin extrusion), MFM32 (Motor Function Measure-32 item), MMRM (Mixed Model Repeated Measures), MRI (magnetic resonance imaging), NCI CTCAE (National Cancer Institute Common Terminology Criteria for Adverse Events), NIMP (non-investigationalmedicinal product), NONMEM (nonlinear mixed effects modeling (software)), NSAA (North Star Ambulatory Assessment), ObsRO (observer-reported outcome), OLE (open-label extension), OTC (over-the-counter), PD (pharmacodynamic), PROMIS (Patient-Reported Outcomes Measurement Information System), PK (pharmacokinetic), QTcB (QT interval corrected through use of Bazett's formula), QTcF (QT interval corrected through use of Fridericia's formula), RHS (Revised Hammersmith Scale), SAD (single ascending dose), SAP (statistical analysis plan), SMA (spinal muscular atrophy), SMAIS (SMA Independence Scale), SMN (survival of motor neuron), SMN1 (Survival of motor neuron 1 (gene)), SMN2 (Survival of motor neuron 2 (gene)), SMNΔ7 (SMN2mRNA that does not contain exon 7), SPA (statistical programming and analysis), TGF-β (transforming growth factor β), ULN (upper limit of normal), VAS (visual analogue scale)

### Example 1: Mouse Model of SMA (Type II/III)

The mouse model of SMA (pharmacological model) that has been used in the preclinical trial herein is the delta7 mice treated with a sub-maximal dose of the SMN-upregulating compound SMN-C1 as described in Naryshkin et al, Science 345, 688-693, 2014. This treatment results in mice that live into adulthood, have reduced SMN levels, and exhibit an SMA-like neuromuscular phenotype (Z. Feng et al., Hum Mol Genet 25, 964-975 2016). The advantage of this model is that it has a more severe phenotype than existing mild models but lives into adulthood, which enables testing SMA treatments after disease onset. In this study, three different versions of the pharmacological model was used to show that GYM329 in combination with SMN-C1 results in an increase in muscle mass and function. (1) a monotherapy, (2) as a combination therapy with an SMN-upregulating compound administered at the onset of disease, and (3) in a mild model of SMA was don. Dosing of the myostatin inhibitor began at PND24 and continued for 28 days until PND52. In all of these models, GYM329 was administrated once per week at 3 mg/kg (4 subcutaneous injections). A cohort of wild type (WT) littermates treated with the vehicle was included as a reference group. At the study's conclusion, in vivo muscle force frequency, muscle weight, muscle histopathology, bone morphometry, and voluntary running were assessed.

### Methods

### Experimental Design

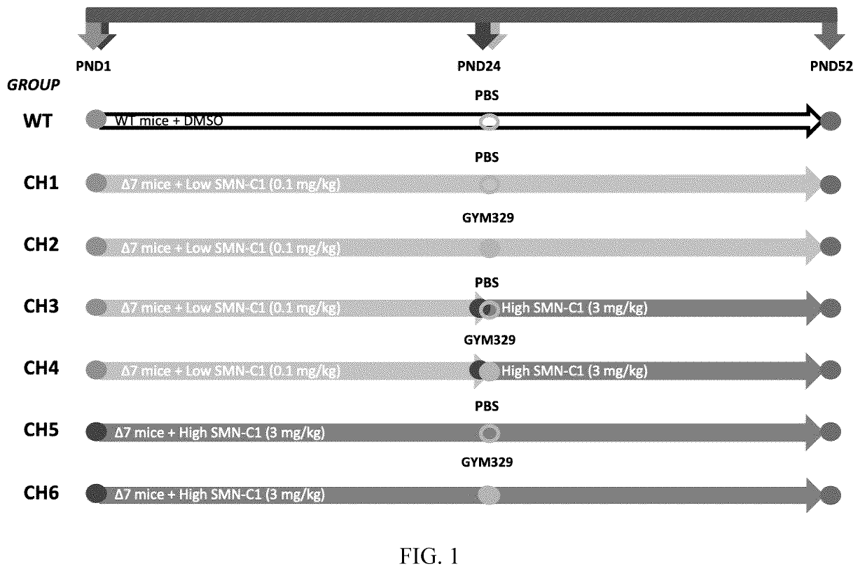

The cohorts examined in this study are depicted in FIG. 1. The study vehicles and treatment are described in Table 3

TABLE 3
Group Designation and Dose Levels

| | | | SMN-C1 | SMN-C1 | |
|---|---|---|---|---|---|
| | | | Dose PND1- | Dose PND24- | |
| | | | PND23 | PND52 | |
| | | | (mg/kg | (mg/kg | Myostatin |
| Group | Mice | Enrollment | IP QD) | IP QD) | Inhibitor Dose |
| WT | WT | 11 | Vehicle | Vehicle | Myostatin Vehicle |
| | | | (DMSO) | (DMSO) | PND 24, 31, 38, 45 |
| CH1 | Δ7 | 10 | 0.1 | 0.1 | Myostatin Vehicle |
| | | | | | PND 24, 31, 38, 45 |
| CH2 | Δ7 | 11 | 0.1 | 0.1 | Myostatin inhibitor |
| | | | | | (3 mg/kg GYM329) |
| | | | | | PND 24, 31, 38, 45 |
| CH3 | Δ7 | 10 | 0.1 | 3.0 | Myostatin Vehicle |
| | | | | | PND 24, 31, 38, 45 |
| CH4 | Δ7 | 10 | 0.1 | 3.0 | Myostatin inhibitor |
| | | | | | (3 mg/kg GYM329) |
| | | | | | PND 24, 31, 38, 45 |
| CH5 | Δ7 | 15 | 3.0 | 3.0 | Myostatin Vehicle |
| | | | | | PND 24, 31, 38, 45 |
| CH6 | Δ7 | 14 | 3.0 | 3.0 | Myostatin inhibitor |
| | | | | | (3 mg/kg GYM329) |
| | | | | | PND 24, 31, 38, 45 |

### Animal Model

The pharmacological model was generated by treating delta7 mice [FVB.Cg-Tg(SMN2*delta7)4299Ahmb Tg(SMN2)89Ahmb, homozygous knockout mice completely lacking mouse Smnl] with a dose of an SMN-upregulating compound, SMN-C1. For CH1 and CH2, the mice were given a suboptimal dose of SMN-C1 (0.1 mg/kg daily) for the duration of the study. For CH3 and CH4, mice were given a suboptimal dose of SMN-C1 (0.1 mg/kg daily) from PND1-23 and switched to a higher dose (3 mg/kg daily) from PND24-52. For CH5 and CH6, mice were treated with a higher dose of SMN-C1 (3 mg/kg daily) for the duration of the study. Wild type (WT) mice [FVB.Cg-Tg(SMN2*delta7)4299Ahmb Tg(SMN2)89Ahmb, homozygous for the mouse Smnl gene] were used as a reference.

### Drug Treatment

Details about SMN upregulation and treatment with anti-myostatin therapy are shown in Table 4.

TABLE 4
Dosing Solutions Volume and Concentrations

| | | | Dosing | Dosing Solution |
|---|---|---|---|---|
| | | | Volume | Concentration |
| Compound | Dose | Vehicle | (Ml/kg | (mg/mL) |
| SMN-C1 | 0.1 mg/kg | 100% | 2.5 | 0.04 |
| | (IP, QD) | DMSO | | |
| SMN-C1 | 3.0 mg/kg | 100% | 2.5 | 1.2 |
| | (IP, QD) | DMSO | | |
| Myostatin | 3 mg/kg | PBS | 1 | 3 |
| inhibitor, | (SQ, | | | |
| GYM329 | weekly) | | | |

### In Vivo Muscle Function

Hind limb muscle performance was measured in vivo with a 305C muscle lever system (Aurora Scientific Inc., Aurora, CAN). Mice were anesthetized via inhalation (˜4-5% isoflurane, or to effect) and placed on a thermostatically controlled table where anesthesia was maintained via nose-cone (˜2% isoflurane, or to effect). Hair was removed from the lower leg by application of depilatory cream for 3 minutes, followed by thorough rinsing

with physiological buffer. The leg was then wiped with a 5% povidone-iodine solution followed by 70% isopropyl alcohol. The knee was isolated using a pin through the tibial head and the foot firmly fixed to a footplate on the motor shaft. Contractions were elicited in the gastrocnemius by percutaneous electrical stimulation of the sciatic nerve.

To assess masseter function, the mouse was anesthetized via inhalation (˜4-5% isoflurane, or to effect) and placed on a thermostatically controlled table where anesthesia was maintained via nose-cone (˜2% isoflurane, or to effect). The mouse was placed supine with custom designed restraint to ensure accessibility of the jaw for testing. The lever arm of the 305C muscle lever system (Aurora Scientific Inc., Aurora, CAN) was positioned in the diastema of the mandible and a suture was run under the middle lower incisors of the mouse and attached to the lever arm. The masseter was contracted by electrical stimulation using surface electrodes.

On both muscles, a force frequency response was performed. Briefly, a series of stimulations were performed at increasing frequency of stimulation (0.2 ms pulse, 500 ms train duration): 1, 10, 20, 40, 60, 80, 100, and 150 Hz, followed by a final stimulation at 1 Hz.

### Voluntary Running Wheel Performance

Starting PND45, mice were housed in living chambers with running wheels for 7 days. Each cage is designed for one mouse and measures 8.4″ wide, 14.25″ long and 5.6″ high, with a running wheel with a 5″ diameter that turns with less than 3 grams of force, allowing the mouse to run with ease and comfort. The wheel is equipped with an electronic counter connected to a computer interface for continuous monitoring of activity. Animals were allowed to run for one week prior to endpoint in vivo muscle function and euthanasia.

### Histology and Muscle Fiber Typing

Plantarflexor (gastrocnemius, plantaris, and soleus) samples frozen for histology were embedded in cryomatrix on a soft cork surface to enable easy sectioning. Briefly, frozen and embedded tissues were mounted in a cryotome and serially sectioned (10 μm thickness) perpendicular to the fiber axis. Multiple slices (5-10) were taken at different portions of the muscle. The slices were then fixed in ice-cold paraformaldehyde and stored at −80° C. until further use.

For cross-sectional area determination, fixed sections from the mid-belly of the muscle were stained with wheat germ agglutinin (WGA) conjugated to a fluorophore to visualize cell membranes. Sections were digitized using fluorescent microscopy, cell boundaries were traced using predictive software, and cross-sectional area was determined via unbiased automated measurements. For muscle fiber type determination, histological slices were taken from the mid-belly of the soleus and the gastrocnemius muscle. The fixed tissue sections were then blocked using SuperBlock PBS blocking buffer (Thermo Fisher) for 1 h at room temperature. The slide was then washed with PBS and covered with a primary antibody against either MyHC-I, MyHC-IIa, or MyHC-IIb (1:20 dilution; Developmental Studies Hybridoma Database) and incubated overnight at 4° C. The slide was then washed with PBS and the appropriate secondary antibody added for 1 h at room temperature. The slide was washed again in PBS, covered in mounting solution, and a coverslip used to seal the tissue section for fluorescence microscopy measures. Fluorescently-labeled tissue sections were digitized using a fluorescence microscope (Nikon). Images were then analyzed for number of cells using standard counting software.

### Data and Statistical Analysis

Muscle function data were analyzed using Aurora Scientific 615A Dynamic Muscle Analysis Software Suite in high-throughput mode. The software automatically determines a baseline, a maximum, and a minimum. The baseline is then subtracted from the maximum to give the maximal generated force. Each data file was manually inspected to ensure that cursors and fits were assigned properly and corrected when necessary. Data were then grouped and means and standard error of the mean (SEM) calculated. In the plantarflexor muscle group, results were presented as torque, expressed in the unit mN·m, since the assay measures the force produced by the gastrocnemius rotating about the ankle. Force was directly measured in the masseter, expressed in the unit gram.

Statistical analysis was performed using SigmaPlot v11. Muscle performance data were analyzed using a two-way repeated measures ANOVA. Post-hoc analysis for pairwise comparisons was performed with a Holm-Sidak test. Body weights, muscle weights, muscle fiber typing data, and cortical and trabecular bone data were analyzed using a one-way ANOVA, with post-hoc analysis performed with a Holm-Sidak test. Data

are presented as means±SEM.

### Results:

### a) SMA Monotherapy: Low Dose SMN-C1 PND1-52, Cohort 1 And Cohort 2

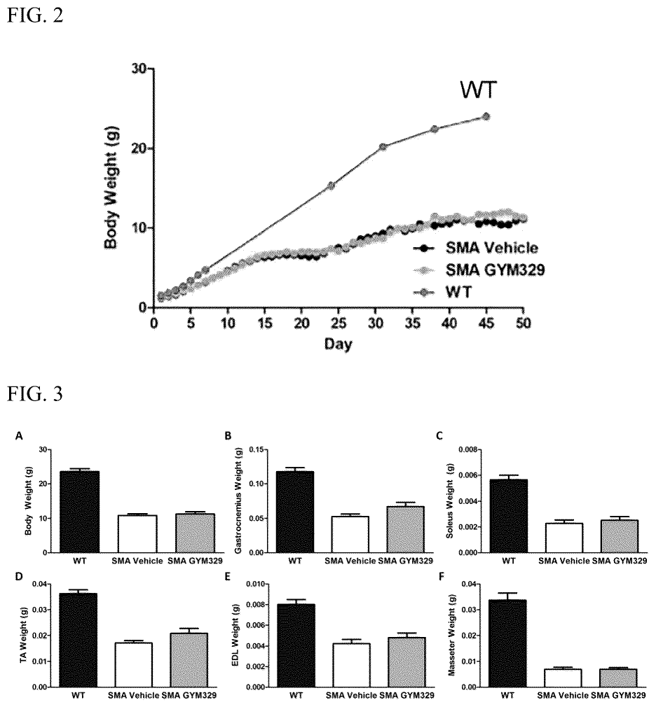

Body weights were taken daily for SMA mice and weekly for WT mice. The growth curves are shown in FIG. 2. Body weight was not significantly increased in mice treated with GYM329. Treatment with GYM329 did not significantly affect muscle weights; however, there was a trend for increased gastrocnemius weight in mice treated with GYM329 (p=0.061; FIG. 3).

### Plantarflexor Muscle Function

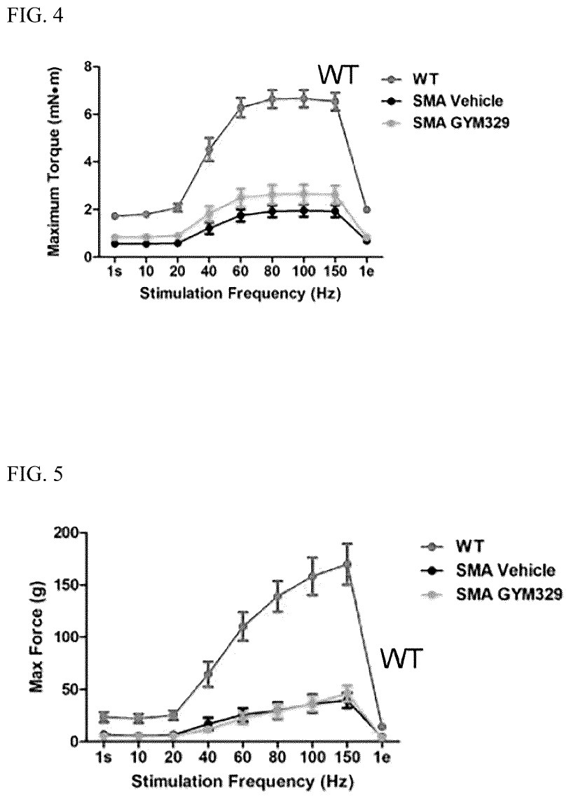

Plantarflexor muscle function was assessed at PND52. There was a trend for improvement with GMY329 treatment (main effect: p=0.078) compared to SMA Vehicle (FIG. 4).

### Masseter Muscle Function

The masseter is a vulnerable muscle in SMA, and its performance was measured at PND52. There was no effect of GYM329 on masseter function compared to SMA Vehicle mice (FIG. 5).

### Muscle Cross-Sectional Area and Fiber Typing

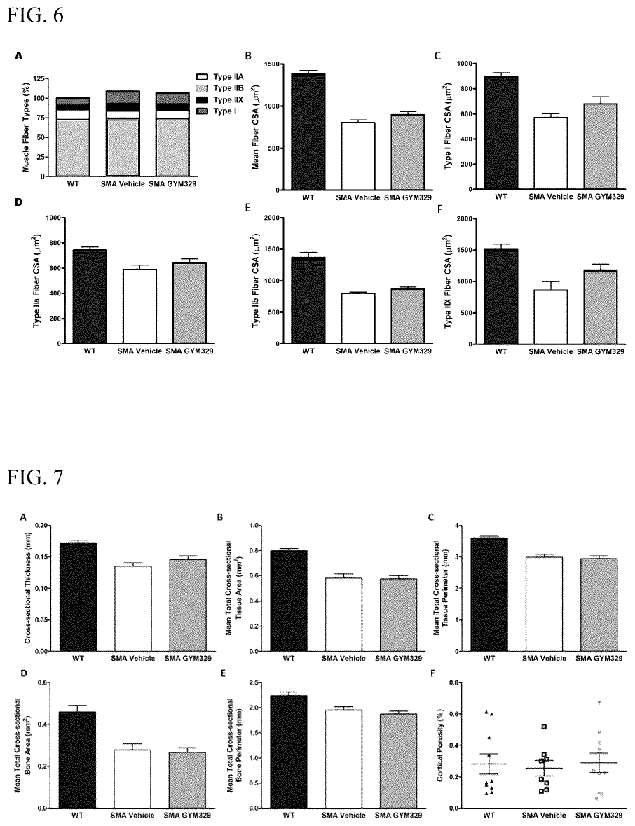

Histology was performed to assess muscle fiber types and cross-sectional area (CSA). There were no differences in muscle fiber type percentages or muscle fiber cross-sectional area with treatment (FIG. 6).

### Bone MicroCT: Tibia

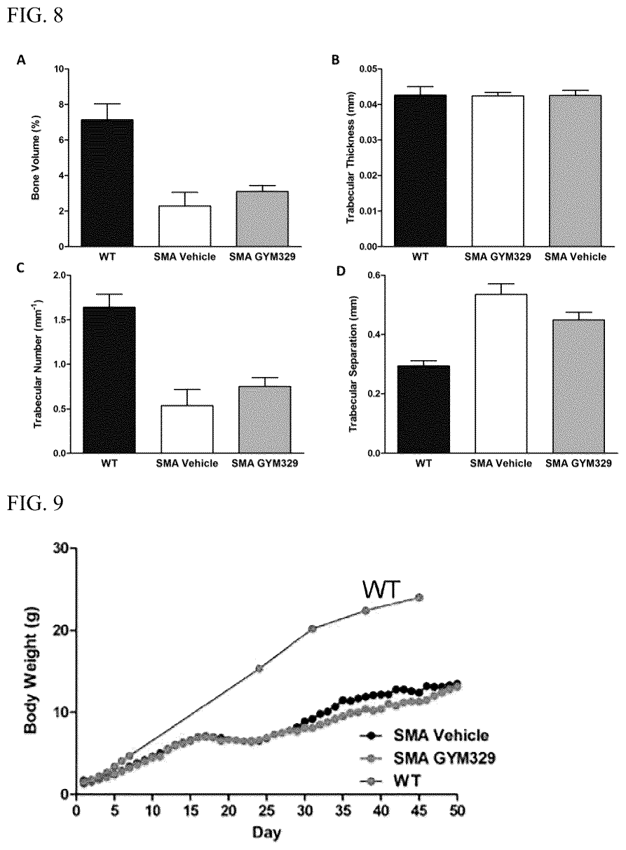

Bone microCT scans were performed in tibias collected at PND52. There was no effect of treatment on cortical or trabecular parameters (FIG. 7 & FIG. 8).

### b) SMA Combination Therapy: Low Dose SMN-C1 PND1-23, High Dose SMN-C1 PND24-52, Cohort 3 And Cohort 4

### Visual Observations: Body Weight and Muscle Weights

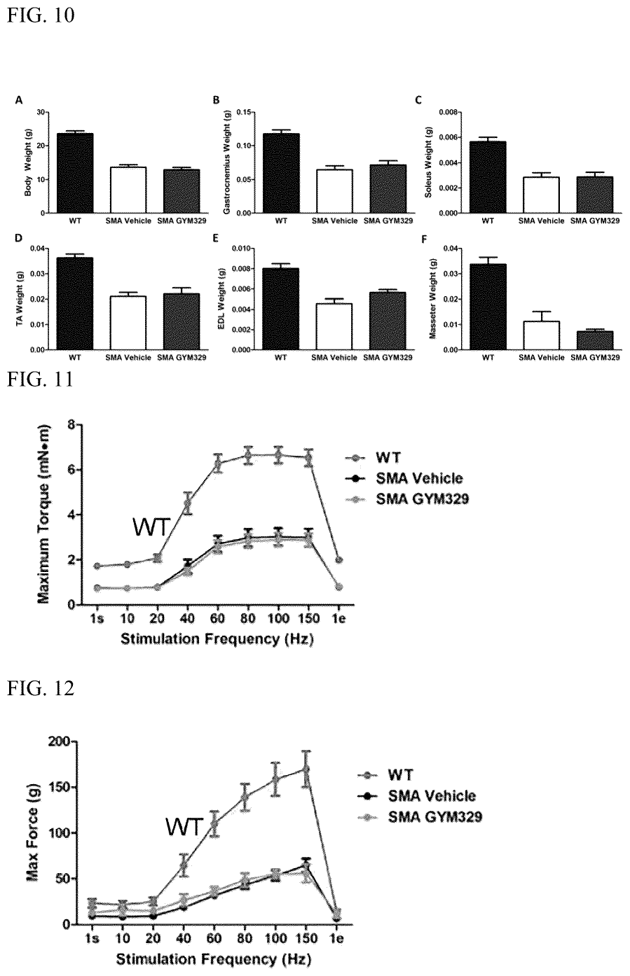

Body weights were measured daily for SMA mice and weekly for WT mice. The growth curves for the mice are shown in FIG. 9. Muscle weights were not affected by treatment (FIG. 10).

### Plantarflexor Muscle Function

Plantarflexor muscle function was assessed at PND52. There was no effect of treatment on plantarflexor muscle function (FIG. 11).

### Masseter Muscle Function

Masseter function was assessed at PND52. There was no effect of treatment on masseter function in these mice (FIG. 12).

### Muscle Cross-Sectional Area and Fiber Typing

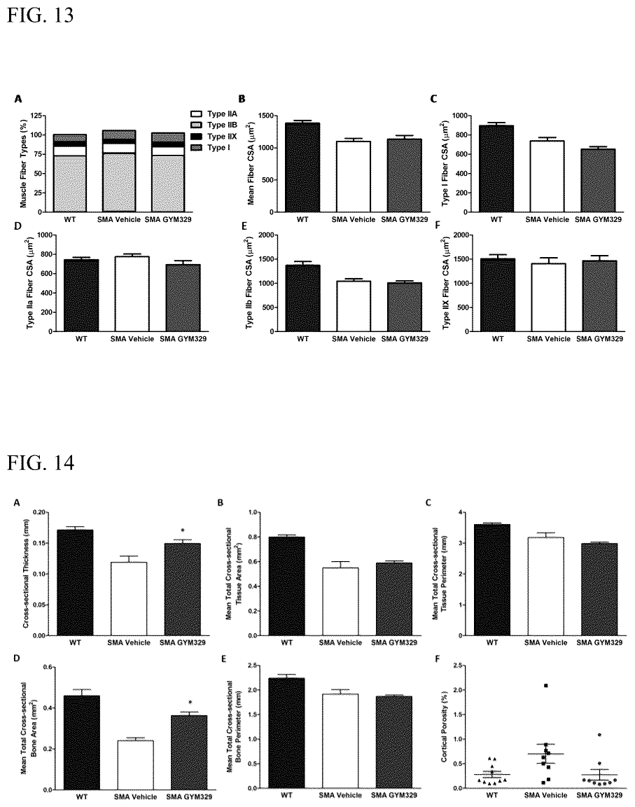

Histology was performed on mounted plantarflexor muscle slices to assess muscle fiber types and CSAs. Muscle fiber type composition and muscle fiber cross-sectional area were not affected by treatment (FIG. 13).

### Bone MicroCT: Tibia

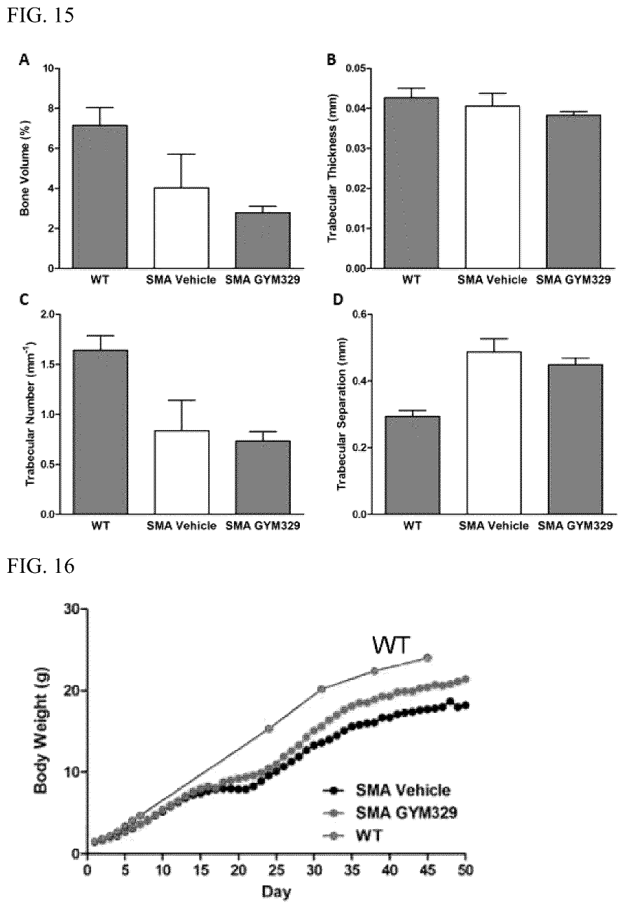

Bone microCT scans of the tibia showed that GYM329 improved cross-sectional thickness and mean total cross-sectional bone area in cortical bone (FIG. 14). There was no effect of treatment on trabecular parameters (FIG. 15).

### c) SMA Combination Therapy (Mild Model): High Dose SMN-C1 PND1-52 Cohort 5 and Cohort 6

### Visual Observations: Body Weight and Muscle Weights

Body weights were taken daily for SMA mice and weekly for WT mice. Their growth curves are shown in FIG. 16. At the of the study, body weights, gastrocnemius weight, soleus weight, and TA weight were significantly increased with GYM329 treatment (FIG. 17).

### Plantarflexor Muscle Function

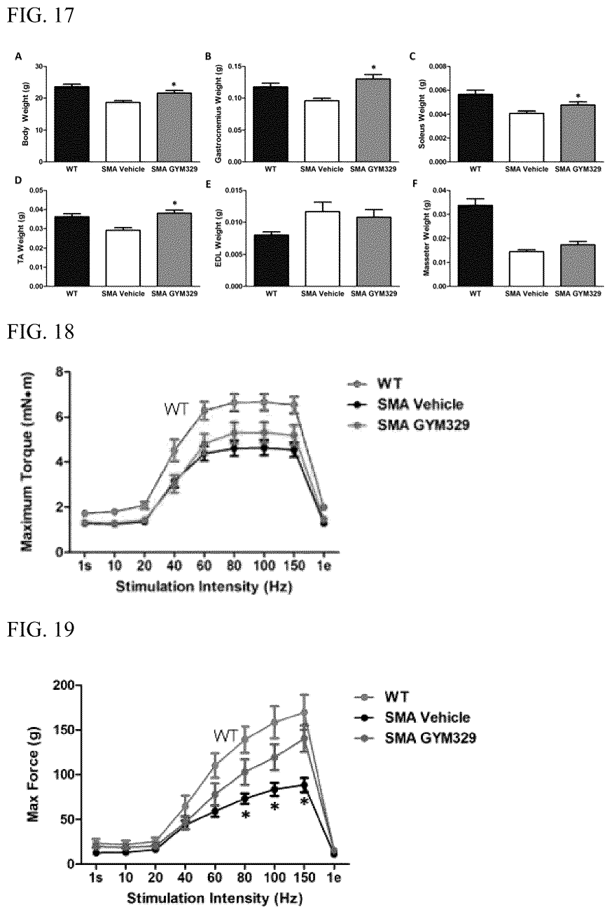

At PND52, plantarflexor muscle function was assessed, and it was not significantly affected by GYM329 treatment (FIG. 18).

### Masseter Muscle Function

Masseter function was improved by GYM329 treatment (main effect: p=0.048 vs. SMA Vehicle). Pairwise comparisons by the Holm-Sidak method showed that at 80, 100, and 150 Hz, maximum force was increased by GYM329 treatment compared to SMA Vehicle (FIG. 19).

### Muscle Cross-Sectional Area and Fiber Typing

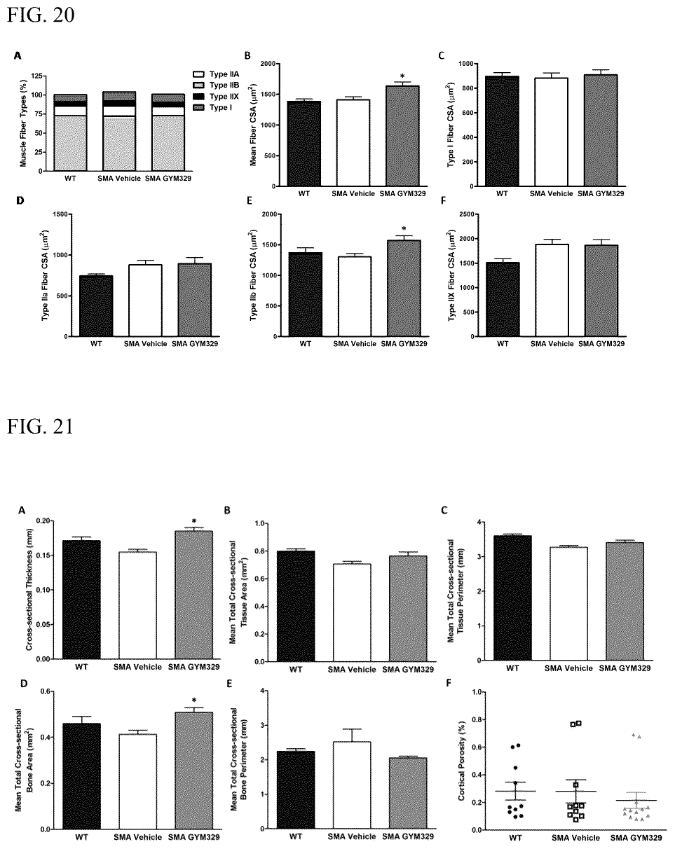

Histology was performed on plantaflexor muscle slices to assess muscle fiber type and cross-sectional area. There were no significant differences in muscle fiber types with treatment; however, GYM329 treatment increased mean fiber and type IIB fiber cross-sectional area compared to SMA Vehicle mice (FIG. 20).

### Bone MicroCT: Tibia

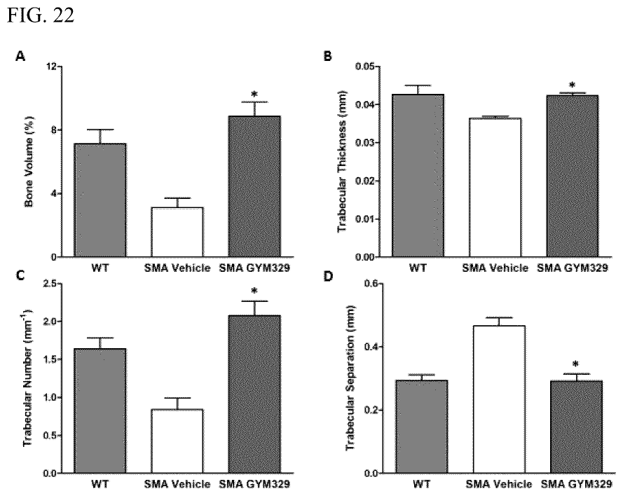

In the tibia, cortical and trabecular bone parameters were assessed. GYM329 treatment improved cortical cross-sectional thickness and mean total cross-sectional bone area (FIG. 21), as well as bone volume, trabecular thickness, trabecular number, and trabecular separation (FIG. 22).

According to the preclincial results describe in a) it seems to suggest that GYM329 as single mean of treatment may not have the desired outcome on SMA patient. According to the results describe in b), it seems that GYM329 without an efficacious treatment of a splicing modifier may not have the desired outcome on SMA patient.

The results of c), indicate strongly that the pretreatment with an efficacious dose of SMN splicing modifier followed by GYM329 treatment leads to unexpected outcome for SMA patient. As shown in part a) GYM329 alone would have little effect on the muscles.

In conclusion, this study indicates a potential benefit of GYM329 in SMA. The strongest effects of GYM329 were observed in the milder SMA model, indicating that a prior rescue of innervation with SMN upregulation treatment before combination treatment with GYM329 is beneficial.

### Example 2

A Phase II/III study, 2-part, open label study will be carried out to investigate the safety, tolerability, pharmacokinetics, pharmacodynamics and efficacy of risdiplam in combination with GYM329 in ambulant pediatric participants (2-10 years of age) with SMA.

The study consists of two parts:

### Part 1: An Exploratory Dose-Finding Part

Part 1 is a double-blind, randomized, placebo-controlled, exploratory study to assess the safety, pharmacokinetics, and pharmacodynamics of GYM329 in combination with risdiplam in ambulant pediatric participants (2-10 years of age) with SMA and to determine the dose for Part 2 of the study. Efficacy of the combination treatment will be assessed as an exploratory objective.

Part1 of the study will enroll approximately 36 participants, with 6 participants aged 2-4 years, and 30 participants aged 5-10 years. Participants naïve to risdiplam will be treated with risdiplam for at least 8 weeks in the run-in period before randomization (2:1, GYM329+risdiplam:placebo+risdiplam) for the 24-week double-blind, placebo-controlled treatment period. Participants treated with risdiplam for at least 8 continuous weeks immediately prior to joining this study maybe randomized to combination therapy immediately or join the run-in period and continue to receive risdiplam monotherapy until randomization, as needed to complete the number of patients required in the cohorts.

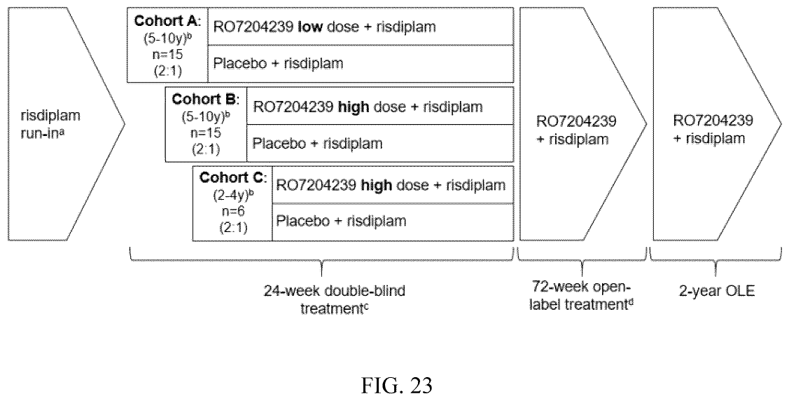

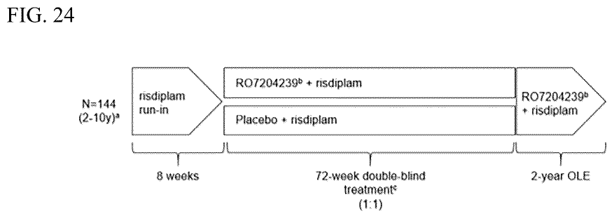

In participants aged 5-10 years, two GYM329 doses (7.4 mg [BW<20 kg] or 10.6 mg [BW ≥20 kg] [low dose] and 24 mg [BW<20 kg] or 36 mg [BW≥20 kg] [high dose]) will be investigated using a staggered, dose-escalation strategy to ensure safe conduct of the study in this pediatric population, as shown in FIG. 23. The high dose is predicted to result in a median steady-state AUC (area under the concentration-time curve) over one dosing interval of 4 weeks of 385 μg×d/mL, which was shown to be well-tolerated in a study in healthy adult subjects, and is expected to achieve close-to-complete inhibition of total and free latent myostatin and mature myostatin.

Participants aged 5-10 years will be randomized initially to the GYM329+risdiplam or placebo+risdiplam low-dose study treatment (Cohort A). Once safety, tolerability, pharmacokinetics,and pharmaco dynamics have been confirmed over at least one dosing interval (after participants have received at least the first 2 blinded doses of GYM329) in all participants in Cohort A and randomization for Cohort A is complete, randomization to the GYM329+risdiplam or placebo+risdiplam high-dose study treatment (Cohort B) may start. The GYM329 dose for Cohorts A, B and Cmax be adjusted from the currently predicted dose levels above, based on emerging safety, PK, and PD data in Part 1. The aim for Cohort B and C is to select a dose that achieves at least 90% inhibition of serum total and free latent myostatin and mature myostatin.

Participants aged 2-4 years will only be randomized once both the low dose and the high dose are shown to be safe and well-tolerated over at least one dosing interval in participants aged 5-10 years, randomization for Cohort B is complete, and all participants in Cohort B have received at least the first two blinded doses of GYM329. The younger participants will then be randomized to GYM329+risdiplam or placebo+risdiplam in CohortC, and the dose for Cohort C will be selected with the aim to achieve similar PK (and PD) as at the high-dose (Cohort B) in the older patients age 5-10 years.

When all participants from Cohort A and Cohort B have completed 24 weeks of therapy in the double-blind period, and PK data over at least one dosing interval is available from all participants in Cohort C (all participants in Cohort C have received the first 2 blinded doses of GYM329), an IMC will review all available safety, tolerability, PK, and PD data to select the GYM329 dose for Part 2 (pivotal dose). If the pivotal dose selected by the IMC is different to that of Cohort C, then participants receiving GYM329 in this cohort will be switched in a blinded manner to the selected pivotal dose for the remainder of the 24-week double-blind period.

Once Part 1 participants have completed the 24-week double-blind treatment period, all participants in Part 1 will receive the GYM329+ risdiplam combination therapy for 72 weeks as part of an open-label treatment period, as illustrated in Section 1.2. If a participant reaches the end of the 24-week double-blind treatment period and the pivotal dose has not yet been determined, the participant will receive GYM329 at the dose of their respective treatment cohort until the pivotal dose has been decided. Once the pivotal dose is selected, all Part 1 participants will then be switched to this pivotal dose. Upon completion of the open-label combination treatment period, participants will have the option to enter the open-label extension (OLE) period where all participants will receive combination treatment for an additional 2 years, unless the development of the combination therapy is stopped.

The duration of the study for each participant enrolled in Part 1 will be divided as follows:
Screening: Study Day −30 to Day −2
Enrollment: Run-in Day −1. Only applicable for patients participating in the risdiplam run-in period
Risdiplam run-in period: Run-in Day 1 to randomization (a minimum of 8 weeks for participants naïve to risdiplam; participants treated with risdiplam for at least 8 continuous weeks immediately prior to joining this study may be randomized to combination therapy immediately or join the run-in period and continue to receive risdiplam monotherapy until randomization, as needed to complete the number of patients required in the cohorts)
Baseline (start of combination therapy): Baseline Day 1
Combination treatment period: 24 weeks (double-blind period)+72 weeks (open-label treatment period)
Open-label extension period: 2 years
Safety Follow-Up: 3 months after final dose of combination treatment.

TABLE 5
Objectives and Corresponding Endpoints for Part 1

| | |
|---|---|
| Primary Objective | Corresponding Endpoint |

Selection of the GYM329 dose for Part 2 based on safety, PK and PD data through:

| Evaluation of the safety of | Incidence, severity, and causal relationship of AEs, |
|---|---|
| GYM329 in combination with | with severity determined according to NCI CTCAE v5.0 |
| risdiplam compared with placebo | Change from baseline in vital signs, physical findings, |
| in combination with risdiplam | ECG, echocardiogram, relevant echocardiographic |
| | parameters and clinical laboratory results |
| | Incidence of local and systemic injection reactions |
| | Incidence of abnormal laboratory findings |
| | Incidence of abnormal ECG parameters |
| | Incidence of relevant echocardiographic parameter |
| | zscores > 2 |
| | Incidenceof abnormal vital signs |
| Evaluation of PK parameters | Serum concentration of GYM329 at specified timepoints |
| for GYM329 when administered | Cₘₐₓ |
| in combination with risdiplam | AUC |
| | C_{trough} |
| | Other PK parameters, as appropriate |
| Evaluation of PK parameters | Plasmaconcentration of risdiplam (and its metabolite M1) |
| for risdiplam when administered | at specified timepoints |
| in combination with GYM329 | Cₘₐₓ |
| | AUC |
| | C_{trough} |
| | Other PK parameters, as appropriate |
| Evaluation of the immune | Prevalence of ADAs at baseline and incidence of ADAs |
| response to GYM329 in | during the study |
| combination with risdiplam | |
| Evaluation of the PD effects | Change from baseline in serum concentrations of total |
| of GYM329 in combination with | and free latent myostatin, and mature myostatin |
| risdiplam compared with placebo | Change from baseline in the contractile area of skeletal |
| in combination with risdiplam | muscle inthethigh as assessed by MRI (participants ≥ |
| | 5 years) at Week 24 of combination treatment |
| | Change from baseline in the contractile area of skeletal |
| | muscle in the calf as assessed by MRI (participants ≥ |
| | 5 years) at Week 24 of combination treatment |
| Evaluation of GYM329 PK/PD | Relationship between PK/PD endpoints, ADA status, |
| effects | and safety or efficacy |
| Exploratory objectives | Corresponding Endpoint |
| Assessment of PD and efficacy | Change from baseline in the contractile area of skeletal |
| of GYM329 in combination with | muscle inthethigh as assessed by MRI (participants ≥ |
| risdiplam compared with placebo | 5 years) at Week 96 of combination treatment |
| in combination with risdiplam | Change from baseline in the contractile area of skeletal |
| | muscle in the calf as assessed by MRI (participants ≥ |
| | 5 years) at Week 96 of combination treatment |
| | Change from baseline in the fat fraction of thigh muscles |
| | as assessed by MRI (participants ≥ 5 years) at Week 24 |
| | and Week 96 of combination treatment |
| | Change from baseline in the fat fraction of calf muscles |
| | as assessed by MRI (participants ≥ 5 years) at Week 24 |
| | and Week 96 of combination treatment |
| | Change from baseline in whole CSA of thigh muscles |
| | as assessed by MRI (participants ≥ 5 years) at Week 24 |
| | and Week 96 of combination treatment |
| | Change from baseline in whole CSA of calfmuscles as |
| | assessed by MRI (participants ≥ 5 years) at Week 24 |
| | and Week 96 of combination treatment |
| | Proportion of participants with a ≥ 2% increase from |
| | baseline incontractile area of skeletal musclein the thigh |
| | as assessed by MRI (participants ≥ 5 years) at Week 24 |
| | and Week 96 of combination treatment |
| | Proportion of participants with a ≥ 2% increase from |
| | baseline incontractile area of skeletal musclein the calf |
| | as assessed by MRI (participants ≥ 5 years) at Week 24 |
| | andWeek 96 of combination treatment |
| | Change from baseline in MFM D1 + D2 score at Week 96 |
| | of combination treatment |
| | Change from baseline in MFM32 total score at Week 96 |
| | of combination treatment |
| | Change from baseline in the RHS total score at Week 96 |
| | of combination treatment |
| | Change from baseline in the time taken to rise from |
| | floor as measured by RHS Item 25 at Week 96 of |
| | combination treatment |
| | Change from baseline in time takento walk/run 10 |
| | meters as measured by RHS Item 19 at Week 96 of |
| | combination treatment |
| | Change from baseline in the caregiver-reported SMAIS |
| | ambulatory module total score at Week 96 of |
| | combination treatment |
| | Change from baseline in the PROMIS ® Parent Proxy |
| | Fatigue-Short Form 10a score (participants ≥ 5 years) |
| | at Week 96 of combination treatment |
| | Proportion of patients rated by caregivers as no change |
| | or improved in the Caregiver Global Impression of |
| | Change (CaGI-C) Scale at Week 96of combination |
| | treatment. |
| | Proportion of patients rated by caregivers as improved |
| | in the Caregiver Global Impression of Change |
| | (CaGI-C) Scale at Week 96of combination treatment. |
| | Change from baseline in elbow flexion strength as |
| | assessed by myometry (participants ≥ 6 years) at Week |
| | 24 and Week 96 of combination treatment |
| | Changefrom baseline in elbow extension strength as |
| | assessed by myometry (participants ≥ 6 years) at Week |
| | 24 and Week 96 of combination treatment |
| | Change from baseline in shoulder abduction strength as |
| | assessed by myometry (participants ≥ 6 years) at Week |
| | 24 and Week 96 of combination treatment |
| | Change from baseline in knee flexion strength as assessed |
| | by myometry (participants ≥ 6 years) at Week 24 and |
| | Week 96 of combination treatment |
| | Change from baseline in knee extension strength as |
| | assessed by myometry (participants ≥ 6 years) at Week |
| | 24 and Week 96 of combination treatment |
| | Change from baseline in lean muscle mass as assessed |
| | by full-body DXA scan (participants ≥ 5 years) at Week |
| | 24 and Week 96 of combination treatment |
| | Change from baseline in EQ-5D-5L index-based score at |
| | Week 96 of combination treatment |

ADA = anti-drug antibody; AUC = area under the concentration-time curve; Cₘₐₓ = maximum observed concentration; C_{trough} = trough concentration; CSA = cross sectional area; D1 + D2 = Domain 1 + Domain 2; DXA = dual-energy X-ray absorptiometry; EQ-5D-5L = EuroQol 5-Dimension, 5 Level Questionnaire; MFM32 = Motor Function Measure-32 item; MRI = magnetic resonance imaging; NCI CTCAE = National Cancer Institute Common Terminology Criteria for Adverse Events; PROMIS ® = Patient-Reported Outcomes Measurement Information System; PD = pharmacodynamic; PK = pharmacokinetic; RHS = Revised Hammersmith Scale; SMAIS = SMA Independence Scale.

### Criteria for Part 2 Dose Selection

### PDCriteria:

≥90% myostatin suppression in serum (total and free latent myostatin, mature myostatin)
≥2% difference between GYM329+risdiplam and placebo+risdiplam in the change from baseline in contractile area of skeletal muscle in the thigh or calf, as assessed by magnetic resonance imaging (MRI) (patients ≥5 years) after 24 weeks. —If a difference of ≥2% muscle growth is observed with both of the Part 1 doses of GYM329+risdiplam as compared with placebo+risdiplam, then data from dual-energy X-ray absorptiometry (DXA) scans, myometry-related endpoints and myostatin concentrations in serum will be used to assess the bioactivity of the combination.

### Safety Criteria:

No evidence for GYM329-induced cardiac hypertrophy by echocardiogram
No more than 2 patients treated with GYM329 in any cohort experience Grade 3 or higher systemic injection reactions (hypersensitivity, including anaphylaxis), unless clearly not related to study drug
   Part 2: A Confirmatory Pivotal Part, Starting Once the Dose has been Selected Based on Part 1 Data

Part 2 is a double-blind, placebo-controlled, randomized (1:1, GYM329+risdiplam: placebo+risdiplam) study to investigate the efficacy, safety and tolerability, pharmacokinetics, and pharmacodynamics of GYM329 in combination with risdiplam in ambulant pediatric participants (2-10 years of age) with SMA.

Part 2 of the study will enroll approximately 144 participants. No more than 48 participants aged 7-10 years at screening will be enrolled.

Participants in Part 2 will complete an 8-week run-in period of treatment with risdiplam monotherapy, followed by a 72-week double-blind treatment period where patients will be randomized to either GYM329+risdiplam or placebo+risdiplam. Participants randomized to GYM329 will receive GYM329 at the dose selected based on the data obtained in Part 1 of the study (the pivotal dose). Once Part 2 participants have completed the 72-week double-blind treatment period, participants will have the option to rollover into the OLE period where all participants will receive GYM329+risdiplam combination treatment for an additional 2 years, unless the development of the combination therapy is stopped. The study scheme of part 2 can be found in FIG. 24.

The primary efficacy objective of Part 2 of the study is to assess the efficacy of GYM329 in combination with risdiplam in ambulant pediatric participants with SMA, as measured by the change from baseline in the Revised Hammersmith Scale (RHS) total score after 72 weeks of combination treatment. Secondary efficacy outcomes and exploratory endpoints are presented in Table 6.

TABLE 6
Objectives and Corresponding Endpoints for Part 2

| Primary Objective | Corresponding Endpoint |
|---|---|
| Evaluation of the efficacy | Change from baseline in the RHS total score at |
| of GYM329 in combination | Week 72 of combination treatment (Study Week 80) |
| with risdiplam compared | |
| with placebo in combination | |
| with risdiplam | |

Secondary Objectives

| Efficacyand PDObjectives | Corresponding Endpoint |
|---|---|
| Evaluation of the efficacy | Change from baseline in MFM D1 + D2 score at Week |
| and PD of GYM329 in | 72 of combination treatment (Study Week 80) |
| combination with risdiplam | Change from baseline in MFM32 total score at Week 72 |
| compared with placebo | of combination treatment (Study Week 80) |
| in combination with | Change from baseline in the time taken to rise from |
| risdiplam | floor as measured by RHS Item 25 at Week 72 of |
| | combination treatment (Study Week 80) |
| | Change from baseline in the time taken to walk/run |
| | 10 meters as measured by RHS Item 19 at Week 72 of |
| | combination treatment (Study Week 80) |
| | Change from baseline in lean muscle mass as assessed |
| | by full-body DXA scan (participants ≥ 5 years) |
| | at Week 72 of combination treatment |
| Safety Objectives | Corresponding Endpoints |
| Evaluation of the safety | Incidence, severity, and causal relationship of AEs, with |
| of GYM329 in combination | severity determined according to NCI CTCAE v5.0 |
| with risdiplam compared | Change from baseline in vital signs, physical findings, |
| with placebo in | ECG, echocardiogram, relevant echocardiographic |
| combination with | parameters and clinical laboratory results |
| risdiplam | Incidence of local and systemic injection reactions |
| | Incidence of abnormal laboratory findings |
| | Incidence of abnormal ECG parameters |
| | Incidence of relevant |
| | echocardiographic parameter z-scores > 2 |
| | Incidence of abnormal vital signs |
| Pharmacokinetic Objectives | Corresponding Endpoints |
| Evaluation of PK parameters | Serum concentration of GYM329 at specified time points |
| for GYM329 when administered | Cₘₐₓ |
| in combination with | AUC |
| risdiplam | C_{trough} |
| Evaluation of PK parameters | Other PK parameters, as appropriate |
| for risdiplam when | Plasmaconcentration of risdiplam (and its metabolite M1) |
| administered in combination | at specified timepoints |
| | Cₘₐₓ |
| | AUC |
| | C_{trough} |
| | Other PK parameters, as appropriate |
| ImmunogenicityObjective | Corresponding Endpoint |
| Evaluation of the immune | Prevalence of ADAs at baseline and incidence of ADAs |
| response to GYM329 in | during the study; impact of ADA on PK, PD, safety |
| combination with risdiplam | and efficacy parameters |

| Exploratory Objectives | Corresponding Endpoint |
|---|---|
| Evaluation of the efficacy | Change from baseline in stride velocity 95th centile as |
| of GYM329 in combination | measured by a wearable device at week 72 of combination |
| with risdiplam compared | treatment (Study Week 80) |
| with placebo in combination | Change from baseline in the ratio between median stride |
| with risdiplam by evaluation | velocity, and the 95th centile, in the morning (8 am-12 pm) |
| of the participant's | vs afternoon (2-6 pm) as measuredby a wearable device at |
| mobility | Week 72 of combination treatment (Study Week 80) |
| | Change from baseline in wrist vertical accelerationpower |
| | as measured by a wearable device at Week 72 of combination |
| | treatment (Study Week 80) |
| | Change from baseline in angular wrist velocity as measured |
| | by a wearable device at Week 72 of combination treatment |
| | (Study Week 80) |
| Evaluation of the efficacy | Change from baseline in the caregiver-reported SMAIS |
| of GYM329 in combination | ambulatory module total score at Week 72 of combination |
| with risdiplam compared | treatment (Study Week 80) |
| with placebo in combination | Change from baseline in the PROMIS ® Parent Proxy |
| with risdiplam | Fatigue Short Form 10a score (participants ≥ 5 years) |
| | at Week 72 of combination treatment (Study Week 80) |
| | Proportion of patients rated by caregivers as no change |
| | or improved in the Caregiver Global Impression of Change |
| | (CaGI-C) Scale at Week 72 of combination treatment |
| | (Study Week 80). |
| | Proportion of patients rated by caregivers as improved |
| | in the Caregiver Global Impression of Change (CaGI-C) |
| | Scale at Week 72 of combination treatment (Study Week 80). |
| | Change from baseline in elbow flexion strength as |
| | assessed by myometry (participants ≥ 6 years) at Week |
| | 72 of combination treatment (Study Week 80) |
| | Change from baseline in elbow extension strength as |
| | assessed by myometry (participants ≥ 6 years at Week |
| | 72 of combination treatment (Study Week 80) |
| | Change from baseline in shoulder abduction strength as |
| | assessed by myometry (participants ≥ 6 years) at |
| | Week 72 of combination treatment (Study Week 80) |
| Exploratory Objectives | Corresponding Endpoint |
| Evaluation of the efficacy | Change from baseline in knee flexion strength as assessed |
| GYM329 in combination with | by myometry (participants ≥ 6 years) at Week 72 of |
| risdiplam compared with | combination treatment (Study Week 80) |
| placebo in combination with | Change from baseline in knee extension strength as assessed |
| risdiplam (continued) | by myometry (participant ≥ 6 years) at Week 72 of |
| | combination treatment (Study Week 80) |
| Evaluation of the serum PD | Change from baseline in serum concentrations of total and |
| (effects of GYM329 in | free latent myostatin, and mature myostatin |
| combination with risdiplam | |
| Evaluation of GYM329 PK/ | Relationship between PK and selected PD endpoints, ADA |
| PD/ADAeffects | status, and safety or efficacy |
| Evaluation of the health | Change from baseline in EQ-5D-5L index-based score at Week |
| related quality of life of | 72 of combination treatment (Study Week 80) |
| participants treated with | |
| GYM329 in combination | |
| with risdiplam compared | |
| withplacebo in combination | |
| with risdiplam | |

ADA = anti-drug antibody; AE = adverse event; AUC = area under the concentration-time curve; Cmax = maximum observed concentration; Ctrough = trough concentration; D1 + D2 = Domain 1 + Domain 2; DXA = dual-energy X-ray absorptiometry; EQ-5D-5L = EuroQol 5-Dimension, 5 Level Questionnaire; MFM32 = Motor Function Measure-32item; NCICTCAE = National Cancer Institute Common Terminology Criteria for Adverse Events; PD = pharmacodynamic; PROMIS^{â} = Patient-Reported Outcomes Measurement Information System; PK = pharmacokinetic; RHS = Revised Hammersmith Scale; SMAIS = SMA Independence Scale.
Note:
A revision to the primary endpoint for Part 2 may be considered after review of blinded baseline motor function scales dataof Part 1 of the study.

The nature, frequency, severity, and timing of adverse events, serious adverse events, local and systemic injection reactions, vital signs, laboratory parameters, ECGs, and echocardiogram tests will be assessed on a regular basis by an unblinded iDMC.

Blood samples for the assessment of PK, PD, and ADA data will be obtained from all participants.

The duration of the study for each participant enrolled in Part 2 will be divided as follows:
Screening: Study Day −30 to Study Day −2
Enrollment: Run-in Day −1
Risdiplam run-in period: Run-in Day 1 to 56(8 weeks)
Baseline (start of combination therapy): Baseline Day 1
Double-blind combination treatment period: 72 weeks
Open-label extension period: 2 years
Safety Follow-up: 3 months after final dose of combination treatment

A schedule of activities and a sample collection schedule are provided in Table 5 and Table 7.

TABLE 7
Schedule of Activities for Part 1 (Screening through Combination Treatment Week 24)

| | | | Risdiplam | | | | | | | |
|---|---|---|---|---|---|---|---|---|---|---|
| | Scrn. [symbol] | Enroll. | Run-In Period ^{b} | Randomized Combination Treatment Period (24 weeks) | | | | | | |
| | Combination Treatment Week | | | | | | | | | |
| | — | | — | W 1 | | | | W 3 | | W 5 |
| | Study Day | | | | | | | | | |
| | D | Run-in | Run-in | BL ^{c} | BL ^{c} | D | D | D | D | D |
| | −30 to −2 | D −1 | Day 1 | D −1 | D 1 | 5 | 11 | 16 | 21 | 29 |
| Visit Window (days) | — | — | — | — | — | ±1 | ±1 | ±1 | ±1 | ±1 |
| Informed consent [symbol] | x | | | | | | | | | |
| Demographic data | x | | | | | | | | | |
| Medical and SMA | x | | | | | | | | | |
| history & baseline | | | | | | | | | | |
| conditions | | | | | | | | | | |
| Eligibility | | x | | x [symbol] | | | | | | |
| IxRS transaction | | x | | x [symbol] | | | | | | |
| Vital signs ^{g} | x | x | | x | | | | | | |
| Weight | x | | | x | | | | | | |
| Height | x | | | x | | | | | | |
| Complete physical | x | | | x | | | | | | |
| examination ^{h} | | | | | | | | | | |
| Tanner staging ⁱ | | x | | x [symbol] | | | | | | |
| Triplicate 12-Lead | x | | | x | | | | | | |
| ECG | | | | | | | | | | |
| Echocardiogram ^{j} | x | | | x | | | | | | |
| Hematology [symbol] and | x | | | | x | | | | | x |
| blood chemistry [symbol] | | | | | | | | | | |
| Coagulation | x | | | | x | | | | | x |
| Menstrual status ^{m} | x | | | x | | | x | | | x |
| Pregnancy test ^{m} | x | | | x | | | x | | | x |
| Urinalysis (dipstick) [symbol] | x | | | x | | | x | | | x |
| MRI (thigh and calf) [symbol] | | | | x | | | x | | | |
| DXA scan [symbol] | | | | | x | | | x | | |
| Muscle strength | x | | | x | | | | | | |
| (myometry) [symbol] | | | | | | | | | | |
| MFM32 | x | | | | x | | | | | |
| RHS | x | | | | | | | | | |
| SMAIS ambulatory | | | | x | | | | | | |
| module | | | | | | | | | | |
| PROMIS ® Parent | | | | x | | | | | | |
| Proxy Short Form - | | | | | | | | | | |
| Fatigue 10a [symbol] | | | | | | | | | | |
| EQ-SD-5L [symbol] | | | | x | | | | | | |
| CaGI-C | | | | | | | | | | |
| Risdiplam | | | x (daily) | | | | | | | |
| administration [symbol] | | | | | | | | | | |
| Risdiplam dosing | | | x (daily) | | | | | | | |
| diary | | | | | | | | | | |
| RO7204239/placebo | | | | | x | | | | | x |
| administration [symbol] | | | | | x | x | x | x | x | x |
| RO7204239 PK | | | | | x | x | | | | |
| sample (serum) [symbol] | | | | | | | | | | |
| Risdiplam PK sample | | | | | | | | | | |
| (plasma) [symbol] | | | | | | | | | | |
| ADA sample [symbol] | | x | | x [symbol] | | | | | | |
| SMN protein [symbol] | | x | | | x [symbol] | | | | | |
| Myostatin samples [symbol] | | x | | x [symbol] | | x [symbol] | x | x [symbol] | x [symbol] | x [symbol] |
| Clinical genotyping | | | | | | | | | | |
| Biomarker sample | x | | | | | | | | | |
| Previous and | x | x | x | x | x | x | x | x | x | x |
| concomitant | | | | | | | | | | |
| medications [symbol] | | | | | | | | | | |
| Previous and | x | x | x | x | x | x | x | x | x | x |
| concomitant surgeries | | | | | | | | | | |
| and medical | | | | | | | | | | |
| procedures | | | | | | | | | | |
| Adverse events [symbol] | | x | x | x | x | x | x | x | x | x |

| | Randomized Combination Treatment Period (24 weeks) | | | | | | | | | |
|---|---|---|---|---|---|---|---|---|---|---|
| | Combination Treatment Week | | | | | | | | | |
| | W 9 | W 13 | W 17 | W 21 | W 21 | W 22 | W 23 | W 23 | W 24 | |
| | Study Day | | | | | | | | | |
| | D | D | D | D | D | D | D | D | D | D |
| | 57 | 85 | 113 | 141 | 145 | 151 | 156 | 161 | 168 | 169 |
| Visit Window (days) | ±7 | ±7 | ±7 | ±3 | ±3 | ±3 | ±3 | ±3 | ±7 | ±7 |
| Informed consent [symbol] | | | | | | | | | | |
| Demographic data | | | | | | | | | | |
| Medical and SMA | | | | | | | | | | |
| history & baseline | | | | | | | | | | |
| conditions | | | | | | | | | | |
| Eligibility | | | | | | | | | | |
| IxRS transaction | | | | | | | | | | |
| Vital signs ^{g} | x | x | x | x | | | | | x | |
| Weight | | | | | | | | | x | |
| Height | | | | | | | | | x | |
| Complete physical | | | | | | | | | x | |
| examination ^{h} | | | | | | | | | | |
| Tanner staging ⁱ | | | | | | | | | x | |
| Triplicate 12-Lead | | | | | | | | | x | |
| ECG | | | | | | | | | | |
| Echocardiogram ^{j} | | | | | | | | | x | |
| Hematology [symbol] and | | | | | | | | | | x |
| blood chemistry [symbol] | | | | | | | | | | |
| Coagulation | | | | | | | | | | x |
| Menstrual status ^{m} | x | x | x | x | | | | | x | |
| Pregnancy test ^{m} | x | x | x | x | | | | | x | |
| Urinalysis (dipstick) [symbol] | | | | | | | | | x | |
| MRI (high and call) [symbol] | | | | | | | | | x | |
| DXA scan [symbol] | | | | | | | | | | x |
| Muscle strength | | | | | | | | | x | |
| (myometry) [symbol] | | | | | | | | | | |
| MFM32 | | | | | | | | | | x |
| RHS | | | | | | | | | | |
| SMAIS ambulatory | | | | | | | | | x | |
| module | | | | | | | | | | |
| PROMIS ® Parent | | | | | | | | | x | |
| Proxy Short Form - | | | | | | | | | | |
| Fatigue 10a [symbol] | | | | | | | | | | |
| EQ-SD-5L [symbol] | | | | | | | | | x | |
| CaGI-C | | | | | | | | | x | |
| Risdiplam | x (daily) | | | | | | | | | |
| administration [symbol] | | | | | | | | | | |
| Risdiplam dosing | x (daily) | | | | | | | | | |
| diary | | | | | | | | | | |
| RO7204239/placebo | x | x | x | x | | | | | | x |
| administration [symbol] | x | x | x | x | x | x | x | x | | x |
| RO7204239 PM | | | | | | x | x | | | x |
| sample (serum) [symbol] | | | | | | | | | | |
| Risdiplam PK sample | | | | x | | | | | | |
| (plasma) [symbol] | | | | | | | | | | |
| ADA sample [symbol] | x | | x | | | | | | | x |
| SMN protein [symbol] | | | | x | | | | | | |
| Myostatin samples [symbol] | x | x [symbol] | x | x | x [symbol] | x | x [symbol] | x [symbol] | | x [symbol] |
| Clinical genotyping | | | | | | | | | | |
| Biomarker sample | | | | | | | | | | |
| Previous and | x | x | x | x | x | x | x | x | x | x |
| concomitant | | | | | | | | | | |
| medications [symbol] | | | | | | | | | | |
| Previous and | x | x | x | x | x | x | x | x | x | x |
| concomitant surgeries | | | | | | | | | | |
| and medical | | | | | | | | | | |
| procedures | | | | | | | | | | |
| Adverse events [symbol] | x | x | x | x | x | x | x | x | x | x |

| | Combination Treatment Period | | | | | | | | | | | | | |
|---|---|---|---|---|---|---|---|---|---|---|---|---|---|---|
| | Combination Treatment Week | | | | | | | | | | | | | |
| | W 29 | W 33 | W 37 | W 41 | W 45 | W 48 | | W 53 | W 57 | W 61 | W 65 | W 69 | W 72 | |
| | Study Day | | | | | | | | | | | | | |
| | D | D | D | D | D | D | D | D | D | D | D | D | D | D |
| | 197 | 225 | 253 | 281 | 309 | 336 | 337 | 365 | 393 | 421 | 449 | 477 | 504 | 505 |
| Visit Window | ±7 | ±7 | ±7 | ±7 | ±7 | ±7 | | ±7 | ±7 | ±7 | ±7 | ±7 | ±7 | |
| Vital signs ^{g} | | | | | | x | | | | | | | x | |
| Weight | | | | | | x | | | | | | | x | |
| Height | | | | | | x | | | | | | | x | |
| Complete physical | | | | | | x | | | | | | | x | |
| examination ^{h} | | | | | | | | | | | | | | |
| Tanner staging ⁱ | | | | | | x | | | | | | | x | |
| Triplicate 12-Lead | | | | | | x | | | | | | | x | |
| ECG | | | | | | | | | | | | | | |
| Echocardiogram | | | | | | x | | | | | | | x | |
| Hematology ^{k} and | | | | | | | x | | | | | | | x |
| blood chemistry ^{l} | | | | | | | | | | | | | | |
| Coagulation | | | | | | | x | | | | | | | x |
| Menstrual status ^{m} | | | x | | x | | | x | x | x | | x | | |
| Pregnancy test ^{m} | x | | | x | x | x | | x | x | x | x | x | | |
| Urinalysis | | | | | | | | | | | | | | |
| (dipstick) ⁿ | | | | | | | | | | | | | | |
| MRI (thigh and | | | | | | | | | | | | | | |
| calf) ^{o, p} | | | | | | | | | | | | | | |
| DXA scan ^{o, p} | | | | | | | x | | | | | | | |
| Muscle strength | | | | | | | | | | | | | x | |
| (myometry) ^{q} | | | | | | | | | | | | | | |
| MFM32 | | | | | | | | | | | | | | x |
| RHS | | | | | | x | | | | | | | x | |
| SMAIS ambulatory | | | | | | x | | | | | | | x | |
| module | | | | | | | | | | | | | | |
| PROMIS ® Parent | | | | | | x | | | | | | | | |
| Proxy Fatigue - Short | | | | | | | | | | | | | | |
| Form 10a [symbol] | | | | | | | | | | | | | | |
| EQ-5D-5L [symbol] | | | | | | x | | | | | | | x | |
| CaGI-C | | | | | | | | | | | | | x | |
| Risdiplam | x (daily) | | | | | | | | | | | | | |
| administration [symbol] | | | | | | | | | | | | | | |
| Risdiplam dosing | x (daily) | | | | | | | | | | | | | |
| diary | | | | | | | | | | | | | | |
| RO7204239/ | x | x | x | x | x | | x | x | x | x | x | x | | x |
| placebo | | | | | | | | | | | | | | |
| administration ^{t} | | | | | | | | | | | | | | |
| RO7204239 PK | x | | | x | | | | x | | | x | | | x |
| sample (serum) ^{u} | | | | | | | | | | | | | | |
| Risdiplam PK | | | | | | | | | | | | | | |
| sample | | | | | | | | | | | | | | |
| (plasma) ^{s, v} | | | | | | | | | | | | | | |
| ADA sample ^{u, w, x} | x | | | x | | | | x | | | x | | | x |
| SMN protein [symbol] | | | | | | | | | | | | | | |
| Myostatin sample [symbol] | x [symbol] | | | x [symbol] | | | | x [symbol] | | | x [symbol] | | | |
| Clinical genotyping | | | | | | | | | | | | | | |
| Biomarker sample | | | | | | | x | | | | | | | |
| Previous and | x | x | x | x | x | x | x | x | x | x | x | x | x | x |
| concomitant | | | | | | | | | | | | | | |
| medications z | | | | | | | | | | | | | | |
| Previous and | x | x | x | x | x | x | x | x | x | x | x | x | x | x |
| concomitant | | | | | | | | | | | | | | |
| surgeries and | | | | | | | | | | | | | | |
| procedures | | | | | | | | | | | | | | |
| Adverse events # | x | x | x | x | x | x | x | x | x | x | x | x | x | x |

| | Combination Treatment Period | | | | | | | | | | |
|---|---|---|---|---|---|---|---|---|---|---|---|
| | Combination Treatment Week | | | | | | | | Completion/ | | |
| | W 77 | W 81 | W 85 | W 89 | W 93 | W 96 | | OLE | Early WD | | |
| | Study Day | | | | | | | | | | |
| | D | D | D | D | D | D | D | >D | D | D | |
| | 533 | 561 | 589 | 617 | 645 | 672 | 673 | 673 | 1 | 2 | SFU |
| Visit Window | ±7 | ±7 | ±7 | ±7 | ±7 | ±7 | | — | — | — | — |
| Vital signs ^{g} | | | | | | x | | x (Q6M) | x | | |
| Weight | | | | | | x | | x (Q6M) | x | | |
| Height | | | | | | x | | x (Q6M) | x | | |
| Complete physical | | | | | | x | | x (Q12M) | x | | |
| examination ^{h} | | | | | | | | | | | |
| Tanner staging ⁱ | | | | | | x | | x (Q12M) | x | | |
| Triplicate 12-Lead | | | | | | x | | x (Q6M) | x | | |
| ECG | | | | | | | | | | | |
| Echocardiogram | | | | | | x | | x (Q6M) | x [symbol] | | |
| Hematology ^{k} and | | | | | | | x | x (Q6M) | | x | |
| blood chemistry ^{l} | | | | | | | | | | | |
| Coagulation | | | | | | | x | x (Q6M) | | x | |
| Menstrual status ^{m} | x | x | x | x | x | x | | x (Q4W) | x | | x |
| Pregnancy test ^{m} | x | x | x | x | x | x | | x (Q4W) | x | | x |
| Urinalysis | | | | | | x | | x (Q6M) | x | | |
| (dipstick) ⁿ | | | | | | | | | | | |
| MRI (thigh and | | | | | | | | | | | |
| calf) ^{o, p} | | | | | | | | | | | |
| DXA scan ^{o, p} | | | | | | | x | x (Q6M) | | x | |
| Muscle strength | | | | | | x | | x (Q6M) | x | | |
| (myometry) ^{q} | | | | | | | | | | | |
| MFM32 | | | | | | | x | x (Q6M) | | x | |
| RHS | | | | | | | | x (Q6M) | x | | |
| SMAIS ambulatory | | | | | | | | x (Q6M) | x | | |
| module | | | | | | | | | | | |
| PROMIS ® Parent | | | | | | x | | x (Q6M) | x | | |
| Proxy Fatigue - Short | | | | | | | | | | | |
| Form 10a [symbol] | | | | | | | | | | | |
| EQ-5D-5L [symbol] | | | | | | | | x (Q6M) | x | | |
| CaGI-C | | | | | | | | x (Q6M) | x | | |
| Risdiplam | x (daily) | | | | | | | | x [symbol] | x [symbol] | |
| administration [symbol] | | | | | | | | | | | |
| Risdiplam dosing | x (daily) | | | | | | | | x | x | |
| diary | | | | | | | | | | | |
| RO7204239/ | x | x | x | x | x | | x | x (Q4W) | | x [symbol] | |
| placebo | | | | | | | | | | | |
| administration ^{t} | | | | | | | | | | | |
| RO7204239 PK | | | x | | | | x | x (Q6M) | | x | |
| sample (serum) ^{u} | | | | | | | | | | | |
| Risdiplam PK | | | | | | | | | | | |
| sample | | | | | | | | | | | |
| (plasma) ^{s, v} | | | | | | | | | | | |
| ADA sample ^{u, w, x} | | | x | | | | x | x (Q6M) | | x | |
| SMN protein [symbol] | | | | | | | | | | | |
| Myostatin sample [symbol] | | | x [symbol] | | | | | | | | |
| Clinical genotyping | | | | | | | | | | | |
| Biomarker sample | | | | | | | | x (Q6M) | | | |
| Previous and | x | x | x | x | x | x | x | x | x | x | x |
| concomitant | | | | | | | | | | | |
| medications ^{z} | | | | | | | | | | | |
| Previous and | x | x | x | x | x | x | x | x | x | x | x |
| concomitant | | | | | | | | | | | |
| surgeries and | | | | | | | | | | | |
| procedures | | | | | | | | | | | |
| Adverse events ^{aa} | x | x | x | x | x | x | x | x | x | x | x |

ADA = anti-drug antibody; BL = Baseline (visit); CaGI-C = Caregiver Global Impression of Change: D = day; DXA = dual-energy X-ray absorptiometry; Enroll. = Enrollment Visit; EQ-5D-5L = EuroQol 5-Dimension, 5-Level Questionnaire; IxRS = interactive voice or web-based response system; MFM32 = Motor Function Measure-32; MRI = magnetic resonance imaging; OLE = open label extension; PROMIS ® = Patient-Reported Outcomes Measurement Information System, PK = pharmacokinetic; Q6M = every 6 months; Q12M = every 12 months; Q4W = every 4 weeks; RHS = Revised Hammersmith Scale; Scrn. = Screening; SFU = safety follow-up; SMAIS = SMA Independence Scale; SMN = survival of motor neuron; WD = Withdrawal.
^{a} Patients who do not meet the criteria for participation in this study may qualify for one re-screening opportunity (for a total of two screenings in total per patient) at the investigator's discretion.
^{b} Participants naive to risdiplam will be treated with risdiplam for at least 8 weeks in the run-in period before randomization for the 24-week double-blind period. Participants treated with risdiplam for at least 8 continuous weeks immediately prior to joining this study may be randomized to combination therapy immediately or will join the run-in period and continue to receive risdiplam monotherapy until randomization.
^{c} Baseline visit is completed over 2 days. Randomization occurs on Baseline (BL) Day −1.
^{d} Informed consent must be documented before any study-specific screening procedure is performed.
^{e} Only applicable if Enrollment Visit/Run-in period does not apply.
^{f} Randomization call to IxRS.
^{g} Vital signs will include temperature (oral or tympanic), systolic and diastolic blood pressure, respiratory and pulse rate. Vital signs will be taken before blood collection and will be measured in a supine position after 10 minutes' rest. On RO7204239 administration days, the vital signs must be taken pre-RO7204239 administration.
^{h} Includes evaluation of the head, eyes, ears, nose, throat, neck and lymph nodes, and the cardiovascular, dermatologic, musculoskeletal, respiratory, gastrointestinal, and neurologic systems, genitourinary examinations may be performed if clinically indicated.
ⁱ Tanner staging to be conducted in all participants who are 9-10 years of age at screening or following their 9th birthday, if they screened before age 9. Once a participant reaches stage 5, Tanner staging no longer needs to be performed.
^{j} During the withdrawal visit, an echocardiogram should be taken, if this visit occurs 3 months after the last echocardiogram.
^{k} Hematology includes WBC and differential counts (neutrophils, eosinophils, basophils, monocytes, lymphocytes, other cells), RBC count, hemoglobin, hematocrit and platelet count.
^{l} Chemistry panel (serum or plasma) includes bicarbonate, sodium, potassium, chloride, glucose, BUN or urea, creatinine, total protein, albumin, creatine phosphokinase (CPK), phosphate, calcium, total and direct bilirubin, ALP, ALT, AST, urate, LDH, LDL, HDL, total cholesterol and triglycerides.
^{m} Menstrual status should be verified in all pre pubertal female participants in order to evaluate their childbearing potential. All women of childbearing potential will have a serum pregnancy test at screening, within 14 days prior to enrollment (Run-in Day −1) or baseline (for Part 1 participants not completing an enrollment visit). Urine pregnancy tests will be performed prior to each administration of RO7204239/placebo during treatment, and at the treatment discontinuation visit. If a urine pregnancy test is positive, it must be confirmed by a serum pregnancy test.
ⁿ Includes dipstick (pH, glucose, protein, blood). If there are clinically significant positive results in blood of protein, confirmed by repeat dipstick, urine will be sent to the laboratory for microscopy and culture (sediment, RBCs, WBCs, casts, crystals, epithelial cells, bacteria). If there is an explanation for the positive dipstick results (e.g., menstruation), it should be recorded and there is no need to perform microscopy and culture.
^{o} In participants ≥5 years at screening.
^{p} If the images need to be repeated due to quality reasons, this should be done within 1 week for images taken at Baseline and within 2 weeks for images taken at Week 24 or later of combination treatment.
^{q} Muscle strength in participants ≥6 years at screening. Assessments include knee flexion, knee extension, elbow flexion, elbow extension and shoulder abduction.
^{r} The proxy reported version of EQ-5D-5L will be completed by caregivers for all participants Part 1.
^{s} The first dose of risdiplam will be administered at the clinical site. Following this, risdiplam administration is conducted at home, other than on the visit days when riadiplam PK sampling is planned, where risdiplam administration will be on site.
^{t} RO7204239 administration will be conducted at the clinical site; participants will be closely monitored for 6 hours post injection after administrations on BL Day 1, Day 29 and Day 169 and the next administration thereafter, and 2 hours for all subsequent administrations (or longer if deemed necessary by the investigator/site staff).
^{u} On days when RO7204239 is administered, any scheduled RO7204239 PK, PD (myostatin, SMN protein) and/or ADA samples should be taken pre-dose. Three separate samples will be collected for myostatin (free latent myostatin, total latent myostatin and mature myostatin).
^{v} Risdiplam PK samples to be taken pre-dose and post-dose at 2, 4, and 6 hours.
^{w} Some participants with injection reaction may be asked to participate in additional procedures to determine the type of immune reaction. Potential candidates will be identified in consultation with the Medical Monitor. The additional procedures may include a blood sample and skin biopsy. The blood sample will be used to assess ADA titers, serum mast cell tryptase/histamine, and complement activation products (e.g., C3D, C3G). The skin biopsy should be collected in a sterile manner in accordance with local procedures, using a 3 mm punch biopsy instrument and formalin fixation, and will assess mast cell, eosinophil, and neutrophil infiltration.
^{x} If an unscheduled ADA sample is needed, then a matching RO7204239 PK sample will be required as well.
^{y} Myostatin samples only applicable for children ≥6 years.
^{z} Medication (e.g., prescription drugs, over-the-counter drugs, vaccines, herbal or homeopathic remedies, nutritional supplements) used by a participant in addition to protocol-mandated treatment from 30 days prior to initiation of study drug until completion of the study.
^{aa} After informed consent has been obtained but prior to initiation of study drug, only serious adverse events caused by a protocol-mandated intervention should be reported. After initiation of study drug, all adverse events will be reported until completion of the study. After this period, the Sponsor should be notified if the investigator becomes aware of any serious adverse event that is believed to be related to prior exposure to study drug
^{bb} Safety follow-up will occur 3 months after the last combination treatment received by the participant.
^{cc} Final dose of study drug to be administered on day of study completion visit. At the investigator's discretion and if appropriate, study drug may be administered on the day of early withdrawal visit. At completion/early withdrawal visit, risdiplam used and unused study drug bottles must be returned to sites. [symbol]
indicates data missing or illegible when filed

TABLE 8
Schedule of Activities for Part 2 (Screening through Combination Treatment Week 32)

| | | | | | Randomized Combination Treatment Period (72 weeks) | | |
|---|---|---|---|---|---|---|---|
| | | | Risdiplam | | Combination Treatment Week | | |
| | Scrn ^{a} | Enroll | Run-In Period | | W 1 | | W 5 |
| | — | | — | | Study Week | | |
| | −4 to −1 | | 1-8 | | W 9 | | W 13 |
| | Study Day | | | | | | |
| | D −30 | Run-in | Run-in | Run-In | BL ^{b} | BL ^{b} | D |
| | to −2 | D −1 | D 1 to 28 | D 29 to 55 | D −1 | D 1 | 29 |
| Visit Window | — | — | — | — | ±1 | ±1 | ±7 |
| Informed consent ^{c} | x | | | | | | |
| Demographic data | x | | | | | | |
| Medical and SMA history and | x | | | | | | |
| baseline conditions | | | | | | | |
| Eligibility | | x | | | | | |
| IxRS transaction | | x | | | x ^{d} | | |
| Vital signs ^{e} | x | x | | | x | | x |
| Weight | x | | | | x | | |
| Height | x | | | | x | | |
| Complete physical examination ^{f} | x | | | | x | | |
| Tanner staging ^{g} | | x | | | | | |
| Triplicate 12-Lead EGG | x | | | | x | | |
| Echocardiogram ^{h} | x | | | | x | | |
| Hematology ⁱ and blood | x | | | | | x | x |
| chemistry ^{j} | | | | | | | |
| Coagulation | x | | | | | x | x |
| Menstrual status ^{k} | x | | | | x | | x |
| Pregnancy test ^{k} | x | | | | x | | x |
| Urinalysis (dipstick) [symbol] | x | | | | x | | x |
| DXA scan ^{m, n} | | | | | | x | |
| Muscle strength (myometry) [symbol] | x | | | | x | | |
| MFM32 | x | | | | | x | |
| RHS | x | | | | x | | |
| SMAIS ambulatory module | | | | | x | | |
| PROMIS ® Parent Proxy Fatigue - | | | | | x | | |
| Short Form 10a [symbol] | | | | | | | |
| Wearable device ^{p} | | | | x | | | |
| Wearable device user | | | | | x | | |
| experience questionnaire | | | | | | | |
| EQ-5D-SL ^{q} | | | | | x | | |
| CaGI-C | | | | | | | |
| Risdiplam administration [symbol] | | | x (daily) | | | | |
| Risdiplam dosing diary | | | x (daily) | | | | |
| RO7204239/placebo | | | | | | x | x |
| administration ^{s} | | | | | | | |
| RO7204239 PK sample | | | | | | x | x |
| (serum) ^{t} | | | | | | | |
| Risdiplam PK sample | | | | | | | |
| (plasma) [symbol] | | | | | | | |
| ADA sample ^{t, v, w} | | | | | | x | |
| SMN protein ^{t} | | x | | | | | |
| Myostatin sample ^{t} | | x ^{x} | | | | x ^{x} | |
| Clinical genotyping | x | | | | | | |
| Biomarker sample | x | | | | | x | |
| Previous and concomitant | x | x | x | x | x | x | x |
| medications ^{y} | | | | | | | |
| Previous and concomitant | x | x | x | x | x | x | x |
| surgeries and procedures | | | | | | | |
| Adverse events ^{z} | | x | x | x | x | x | x |

| | Randomized Combination Treatment Period (72 weeks) | | | | | |
|---|---|---|---|---|---|---|
| | Combination Treatment Week | | | | | |
| | W 9 | W 13 | W 17 | W 21 | W 24 | |
| | Study Week | | | | | |
| | W 17 | W 21 | W 25 | W 29 | W 32 | |
| | Study Day | | | | | |
| | D | D | D | D | D | |
| | 57 | 85 | 113 | 141 | 168 | D |
| Visit Window | ±7 | ±7 | ±7 | ±7 | ±7 | 169 |
| Informed consent ^{c} | | | | | | |
| Demographic data | | | | | | |
| Medical and SMA history and | | | | | | |
| baseline conditions | | | | | | |
| Eligibility | | | | | | |
| IxRS transaction | | | | | | |
| Vital signs ^{e} | x | x | x | x | x | |
| Weight | | | | | x | |
| Height | | | | | x | |
| Complete physical examination ^{f} | | | | | x | |
| Tanner staging ^{g} | | | | | x | |
| Triplicate 12-Lead EGG | | | | | x | |
| Echocardiogram ^{h} | | | | | x | |
| Hematology ⁱ and blood | | | | | | x |
| chemistry ^{j} | | | | | | |
| Coagulation | | | | | | x |
| Menstrual status ^{k} | x | x | x | x | x | |
| Pregnancy test ^{k} | x | x | x | x | x | |
| Urinalysis (dipstick) [symbol] | | | | | x | |
| DXA scan ^{m, n} | | | | | | x |
| Muscle strength (myometry) [symbol] | | | | | x | |
| MFM32 | | | | | | x |
| RHS | | | | | x | |
| SMAIS ambulatory module | | | | | x | |
| PROMIS ® Parent Proxy Fatigue - | | | | | x | |
| Short Form 10a [symbol] | | | | | | |
| Wearable device ^{p} | | | | x | | |
| Wearable device user | | | | | x | |
| experience questionnaire | | | | | | |
| EQ-5D-SL ^{q} | | | | | x | |
| CaGI-C | | | | | x | |
| Risdiplam administration [symbol] | x (daily) | | | | | |
| Risdiplam dosing diary | x (daily) | | | | | |
| RO7204239/placebo | x | x | x | x | | x |
| administration ^{s} | | | | | | |
| RO7204239 PK sample | x | x | x | x | | x |
| (serum) ^{t} | | | | | | |
| Risdiplam PK sample | | | | | | x |
| (plasma) [symbol] | | | | | | |
| ADA sample ^{t, v, w} | x | | x | | | x |
| SMN protein ^{t} | | | | | | |
| Myostatin sample ^{t} | x ^{x} | | x ^{x} | | x ^{x} | |
| Clinical genotyping | | | | | | |
| Biomarker sample | | | | | x ^{x} | |
| Previous and concomitant | x | x | x | x | x | x |
| medications ^{y} | | | | | | |
| Previous and concomitant | x | x | x | x | x | x |
| surgeries and procedures | | | | | | |
| Adverse events ^{z} | x | x | x | x | x | x |

| | Combination Treatment Period | | | | | | | | | | |
|---|---|---|---|---|---|---|---|---|---|---|---|
| | Combination Treatment Week | | | | | | | | | | |
| | 29 | 33 | 37 | 41 | 45 | 48 | | 53 | 57 | 6% | 65 |
| | Study Week | | | | | | | | | | |
| | 37 | 41 | 45 | 49 | 53 | 56 | | 61 | 65 | 69 | 73 |
| | Study Day | | | | | | | | | | |
| | Day | Day | Day | Day | Day | Day | Day | Day | Day | Day | Day |
| | 197 | 225 | 253 | 28% | 309 | 336 | 337 | 365 | 393 | 421 | 449 |
| Visit Window | ±7 | ±7 | ±7 | ±7 | ±7 | ±7 | | ±7 | ±7 | ±7 | ±7 |
| Vital signs [symbol] | | | | | | x | | | | | |
| Weight | | | | | | x | | | | | |
| Height | | | | | | x | | | | | |
| Complete physical examination ^{f} | | | | | | x | | | | | |
| Tanner staging ^{g} | | | | | | x | | | | | |
| Taplicate 12-Lead EDG | | | | | | x | | | | | |
| Echocardiogram ^{h} | | | | | | x | | | | | |
| Hematology [symbol] and | | | | | | | x | | | | |
| blood chemistry ^{j} | | | | | | | | | | | |
| Coagulation | | | | | | | x | | | | |
| Menstrual status ^{k} | x | x | x | x | x | x | | x | x | x | x |
| Pregnancy test ^{k} | x | x | x | x | x | x | | x | x | x | x |
| Urinalysis (dipstick) [symbol] | | | | | | x | | | | | |
| DXA scan [symbol] | | | | | | | x | | | | |
| Muscle strength (myometry) [symbol] | | | | | | x | | | | | |
| MFM32 | | | | | | | x | | | | |
| RHS | | | | | | x | | | | | |
| SMAIS ambulatory module | | | | | | x | | | | | |
| PROMIS ® Parent Proxy Short | | | | | | x | | | | | |
| Form - Fatigue 10a [symbol] | | | | | | | | | | | |
| Wearable device [symbol] | | | | | x | | | | | | |
| Wearable device questionnaire | | | | | | x | | | | | |
| EQ-5D-5L [symbol] | | | | | | x | | | | | |
| CaGI-C | | | | | | x | | | | | |
| Risdiplam administration [symbol] | x (daily) | | | | | | | | | | |
| Risdiplam dosing diary | x (daily) | | | | | | | | | | |
| RO7204239/placebo | x | x | x | x | x | | x | x | x | x | x |
| administration [symbol] | | | | | | | | | | | |
| RO7204239 PK sample | | | x | | | | x | | | x | |
| (serum) ^{t} | | | | | | | | | | | |
| Risdiplam PK sample | | | | | | | | | | | |
| (plasma) [symbol] | | | | | | | | | | | |
| ADA sample ^{t, v, w} | | | | | | | x | | | | |
| SMN protein ^{t} | | | | | | | | | | | |
| Myostatin sample ^{t} | | | x ^{x} | | | x ^{x} | | | | x ^{x} | |
| Clinical genotyping | | | | | | | | | | | |
| Biomarker sample | | | | | | x ^{x} | | | | | |
| Previous and concomitant | x | x | x | x | x | x | x | x | x | x | x |
| medications [symbol] | | | | | | | | | | | |
| Previous and concomitant | x | x | x | x | x | x | x | x | x | x | x |
| surgeries and procedures | | | | | | | | | | | |
| Adverse events [symbol] | x | x | x | x | x | x | x | x | x | x | x |

| | Combination Treatment Period | | | | | | |
|---|---|---|---|---|---|---|---|
| | Combination Treatment Week | | | | | | |
| | 69 | 72 | | | | | |
| | Study Week | | | | Completion/ | | |
| | 77 | 80 | | OLE | Early WD | | |
| | Study Day | | | | | | |
| | Day | Day | Day | >Day | | | |
| | 477 | 504 | 505 | 505 | Day 1 | Day 2 | SFU [symbol] |
| Visit Window | ±7 | ±7 | | — | — | — | — |
| Vital signs [symbol] | | x | | x (Q6M) | x | | |
| Weight | | x | | x (Q6M) | x | | |
| Height | | x | | x (Q6M) | x | | |
| Complete physical examination ^{f} | | x | | x (Q12M) | x | | |
| Tanner staging ^{g} | | x | | x (Q12M) | x | | |
| Taplicate 12-Lead EDG | | x | | x (Q6M) | x | | |
| Echocardiogram ^{h} | | x | | x (Q6M) | x [symbol] | | |
| Hematology [symbol] and | | | x | x (Q6M) | | x | |
| blood chemistry ^{j} | | | | | | | |
| Coagulation | | | x | x (Q6M) | | x | |
| Menstrual status ^{k} | | x | | x (Q4W) | x | | |
| Pregnancy test ^{k} | | x | | x (Q4W) | x | | |
| Urinalysis (dipstick) [symbol] | | x | | x (Q6M) | x | | |
| DXA scan [symbol] | | | x | x (Q6M) | | x | |
| Muscle strength (myometry) [symbol] | | x | | x (Q6M) | x | | |
| MFM32 | | | x | x (Q6M) | | x | |
| RHS | | x | | x (Q6M) | x | | |
| SMAIS ambulatory module | | x | | x (Q6M) | x | | |
| PROMIS ® Parent Proxy Short | | x | | x (Q6M) | x | | |
| Form - Fatigue 10a [symbol] | | | | | | | |
| Wearable device [symbol] | x | | | | | | |
| Wearable device questionnaire | | x | | | | | |
| EQ-5D-5L [symbol] | | x | | x (Q6M) | x | | |
| CaGI-C | | x | | x (Q6M) | x | | |
| Risdiplam administration [symbol] | x (daily) | | | | x ^{bb} | x ^{bb} | |
| Risdiplam dosing diary | x (daily) | | | | x | x | |
| RO7204239/placebo | | | x | x (Q4W) | | x ^{bb} | |
| administration [symbol] | | | | | | | |
| RO7204239 PK sample | | | x | x (Q6M) | | x | |
| (serum) ^{t} | | | | | | | |
| Risdiplam PK sample | | | | | | | |
| (plasma) [symbol] | | | | | | | |
| ADA sample ^{t, v, w} | | | x | | | x | |
| SMN protein ^{t} | | | | | | | |
| Myostatin sample ^{t} | | x ^{x} | | x (Q6M) [symbol] | | | |
| Clinical genotyping | | | | | | | |
| Biomarker sample | | x ^{x} | | x (Q6M) [symbol] | | | |
| Previous and concomitant | x | x | x | x | x | x | x |
| medications [symbol] | | | | | | | |
| Previous and concomitant | x | x | x | x | x | x | x |
| surgeries and procedures | | | | | | | |
| Adverse events [symbol] | x | x | x | x | x | x | x |

ADA = anti-drug antibody; BL = Baseline; CaGI-C = Caregiver Global Impression of Change: D = day, DXA = dual-energy X-ray absorptiometry; EQ-5D-5L = EuroQol 5-Dimension, 5-Level Questionnaire; IxRS = interactive voice of web-based response system; MFM32 = Motor Function Measure-32; OLE = open label extension, PROMIS ® = Patient-Reported Outcomes Measurement Information System; PK = pharmacokinetic; Q6M = every 6 months; Q12M = every 12 months; Q4W = every 4 weeks; RHS = Revised Hammersmith Scale; SFU = safely follow-up; SMAIS = SMA Independence Scale; SMN = survival of motor neuron; WD = Withdrawal.
Note:
For order of assessments follow guidance in Section 8.
^{a} Patients who do not meet the criteria for participation in this study may qualify for one re-screening opportunity (for a total of two screenings in total per patient) at the investigator's discretion.
^{b} Baseline visit is completed over 2 days after the last day of the risdiplam run-in period. Randomization occurs on Baseline Day −1.
^{c} Informed consent must be documented before any study-specific screening procedure is performed.
^{d} Randomization call to IxRS
^{e} Vital signs will include temperature (oral or tympanic), systolic and diastolic blood pressure, respiratory and pulse rate. Vital signs will be taken before blood collection and will be measured in a supine position after 10 minutes' rest. On RO7204239 administration days, the vital signs must be taken pre-RO7204239 administration
^{f} Includes evaluation of the head, eyes, ears, nose, throat, neck and lymph nodes, and the cardiovascular, dermatologic, musculoskeletal, respiratory, gastrointestinal, and neurologic systems, genitourinary examinations may be performed if clinically indicated.
^{g} Tanner staging to be conducted in al participants who are 9-10 years of age at screening of following their 9th birthday, if they enrolled in the study before age 9. Once a participant reaches stage 5, Tanner staging no longer needs to be performed.
^{h} During the withdrawal visit an echocardiogram should be taken, if this visit occurs 3 months after the last echocardiogram.
ⁱ Hematology includes WBC and differential counts (neutrophils, eosinophils, basophils, monocytes, lymphocytes, other cells)., RBC count, hemoglobin, hematocrit and platelet count,
^{j} Chemistry panel (serum or plasma) includes bicarbonate, sodium, potassium, chloride, glucose, BUN or urea, creatinine, total protein, albumin, creatine phosphokinase (CPK), phosphate, calcium, total and direct bilirubin, ALP, ALT, AST, urate, LDH, LDL, HDL, total cholesterol and triglycerides.
^{k} Menstrual status should be verified in all pre pubertal female participants in order to evaluate their childbearing potential. All women of childbearing potential will have a serum pregnancy test at screening, within 14 days prior to enrollment (Run-in Day −1). Urine pregnancy tests will be performed prior to each administration of RO7204238/placebo during treatment, and at the treatment discontinuation visit. If a urine pregnancy test is positive, it must be confirmed by a serum pregnancy test.
^{l} Includes dipstick (pH, glucose, protein, blood). If there are clinically significant positive results in blood of protein, confirmed by repeat dipstick, urine will be sent to the laboratory for microscopy and culture (sediment, RBCs, WRCs, casts, crystals, epithelial cells, bacteria). If there is an explanation for the positive dipstick results (e.g., menstruation), it should be recorded and there is no need to perform microscopy and culture.
^{m} In participants ≥5 years at screening.
ⁿ If the images need to be repeated due to quality reasons, this should be done within 1 week for images taken at Baseline and within 2 weeks for images taken at Week 24 or later of combination treatment.
^{o} Muscle strength in participants ≥6 years at screening. Assessments include knee flexion, knee extension, elbow flexion, elbow extension and shoulder abduction.
^{p} Participants will wear a wearable device daily on the dominant wrist and the ankle for 4 weeks prior to a site visit for the following: baseline (i.e., the last 4 weeks of the run-in period, Run-in Day 29 to Day 55 inclusive); and Day 141 to 167 (inclusive), Day 309 to Day 335 (inclusive), and Day 477 to Day 503 (inclusive).
^{q} The proxy reported version of the EQ-5D-5L will be completed by caregivers for all participants in Part 2 of the study.
^{r} The first dose of risdiplam will be administered at the clinical site on Run-in Day 1. Following this, risdiplam administration is conducted at home, offer then on the visit days when risdiplam PK sampling is planned, where risdiplam administration will be on site.
^{s} RO7204239 administration will be conducted at the clinical site; participants will be closely monitored for 6 hours post injection after administrations on BL Day 1, Day 29 and Day 169 and the next administration thereafter, and 2 hours for all subsequent administrations thereafter (or longer if deemed necessary by the investigator/site staff).
^{t} On days when RO7204239 is administered, any scheduled RO7204239 PK, PD (myostatin, SMN protein) and/or ADA samples should be taken pre-dose. Three separate samples will be collected for myostatin (free latent myostatin, total latent myostatin and mature myostatin).
^{u} Risdiplam PK samples to be taken pre-dose and post-dose at 2, 4, and 6 hours.
^{v} Some participants with injection reaction may be asked to participate in additional procedures to determine the type of immune reaction. Potential candidates will be identified in consultation with the Medical Monitor. The additional procedures may include a blood sample and skin biopsy. The blood sample will be used to assess ADA titers, serum mast cell tryptase/histamine, and complement activation products (e.g., C3D, C3G). The skin biopsy should be collected in a sterile manner in accordance with local procedures, using a 3 mm punch biopsy instrument and formalin fixation, and will assess mast cell, eosinophil, and neutrophil infiltration.
^{w} If an unscheduled ADA sample is needed, then a matching RO7204239 PK sample will be required as well.
^{x} Myostatin and biomarker samples only applicable for children ≥6 years (at the specific visit)
^{y} Medication (e.g., prescription drugs, over-the-counter drugs, vaccines, herbal or homeopathic remedies, nutritional supplements) used by a participant in addition to protocol-mandated treatment from 30 days prior to screening drug until completion of the study.
^{z} After informed consent has been obtained but prior to initiation of study drug, only serious adverse events caused by a protocol mandated intervention should be reported. After initiation of study drug, all adverse events will be reported until completion of the study. Alter this period, the Sponsor should be notified if the investigator becomes aware of any serious adverse event that is believed to be related to prior exposure to study drug
^{aa} Safety follow-up will occur 3 months after the last combination treatment received by the participant.
^{bb} Final dose of study drug to be administered on day of study completion visit. At the investigator's discretion and if appropriate, study drug may be administered on the day of early withdrawal visit. At completion/early withdrawal visit, risdiplam used and unused study drug bottles must be returned to sites. [symbol]
indicates data missing or illegible when filed

### Dosage

The proposed doses for each cohort in Part 1 of GYM329 are presented in Table 9

TABLE 9
Planned GYM329 Doses in Part 1

| | | | Planned |
|---|---|---|---|
| | | | Injection |
| Treatment | Participant | | Volume |
| Cohort | Age | Planned Dose of RO7204239 | (mL)^{b} |
| A | 5 to 10 | 7.4 mg for body weight < 20 kg | 0.35 |
| | years | 10.6 mg for body weight ≥ 20 kg | 0.50 |
| B | 5 to 10 | 24 mg ^{a} for body weight < 20 kg | 0.30 |
| | years | 36 mg ^{a} for body weight ≥ 20 kg | 0.45 |
| C | 2 to 4 | 24 mg ^{a} for body weight < 20 kg | 0.30 |
| | years | 36 mg ^{a} for body weight ≥ 20 kg | 0.45 |

PD = pharmacodynamics; PK=pharmacokinetics
^{a} This dose may be modified; the final dose for Cohort B and C will be determined based on emerging safety, PK and PD data from Part 1 of this study
^{b}The planned injection volume is obtained after 1 dilution step from a stock of 80 mg/mL described in the Pharmacy Manual; volume to be adjusted depending on the final selected dose

The doses for Cohort A and Cohort B will be investigated in a staggered, dose-escalation manner to ensure a safe conduct of the study in this pediatric patient population. Participants aged 5-10 years will be initially randomized into the GYM329+risdiplam or placebo+risdiplam low-dose study treatment (Cohort A). Once safety, pharmaco kinetics and pharmacodynamics have been confirmed over at least one dosing interval (after receiving at least the first 2 blinded doses of GYM329) in all participants in Cohort A, and randomization for Cohort A is complete, randomization may start into the GYM329+risdiplam or placebo+risdiplam high-dose study treatment (Cohort B).

Participants aged 2-4 years (CohortC) will only be randomized if the doses administered to participants aged 5-10 years in both the low-dose and the high-dose cohorts (Cohorts A and B) are shown to be safe and well-tolerated over at least one dosing interval, randomization for Cohort B is complete, and all participants in Cohort B have received at least the first 2 blinded doses of GYM329. These younger participants will then be randomized into the GYM329+risdiplam or placebo+risdiplam groups in Cohort C, with the aim to achieve the same PK (and PD) as in the high-dose Cohort B in older patients.

Risdiplam will be administered at a dose of 5 mg once daily for participants with a BW ≥20 kg or 0.25 mg/kg for participants with a BW <20 kg, as per the approved dosing regimen.

### Stopping Criteria

### Stopping Rules Criteria in Part 1 of the Study

In Part 1, enrollment into the next scheduled cohort will not occur if either of the following criteria occur in the preceding cohort:
More than 2 patients treated with GYM329 in a given cohort experience Grade 3 or higher systemic injection reactions (hypersensitivity, including anaphylaxis), unless clearly not related to study drug
More than 2 patients treated with GYM329 in a given cohort experience any of the following, unless clearly not related to study drug:
   Grade 3 or higher adverse events of the same type
   Clinically significant abnormal vital signs of the same type
   Clinically significant laboratory abnormalities of the same type
   Clinically significant changes in ECGs of the same type

### Individual Stopping Rules

Participants must permanently discontinue from GYM329 if they experience any of the following:
Grade 3 or higher systemic injection reactions (hypersensitivity, including anaphylaxis)
Grade 3 or higher local injection site reaction

Participants must permanently discontinue from GYM329 and/or risdiplam if they experience any of the following:
Patients with any elevated ALT of >3×ULN, ALP>2×ULN, and associated with an increase in bilirubin (>2×ULN) (i.e., a suspected “Hy's law” which indicates risk of severe/serious liver impairment) in the absence of a different explanation
Significant and clinically relevant changes in laboratory parameters, ECG or vital signs which pose an unacceptable risk for the patient
Other findings such as a serious adverse event or any other severe adverse event that indicate that dosing should be halted
Any medical condition that the investigator or Sponsor determines may jeopardize the participant's safety, if he or she continues to receive study treatment
Investigator or Sponsor determination that treatment discontinuation is in the best interest of the participant

Participants must discontinue both treatments if the following apply:
Ongoing pregnancy
Unable to continue to comply with study requirements.

### End of Study Definition

A participant is considered to have completed the study (Part 1 or Part 2), if he or she has completed all phases of the study, including the last visit shown in the schedule of activities for the study as shown in Table 1.

The end of this study is defined as the date of the last visit of the last participant in the study or the date at which the last data point required for statistical analysis (i.e., for the final analysis) or safety follow-up is received from the last participant in the study, whichever occurs later. The end of the study is expected to occur approximately 4 years after the last participant is enrolled in Part 2.

In addition, the Sponsor may decide to terminate the study at any time.

### Duration of Participation

The total duration of study participation for each individual is expected to be approximately 4 to 4.5 years.

### Study Population

### Part 1:

Approximately 36 ambulant pediatric participants of 2-10 years of age with SMA will be enrolled in this part of the study.

### Part 2:

Approximately 144 ambulant pediatric participants of 2-10 years of age with SMA will be enrolled in this pivotal part of the study. No more than 48 participants aged 7W 10 years at screening will be enrolled.

### Inclusion Criteria

Participants are eligible to be included in the study only if all of the following criteria apply:
Participants who are 2 to 10 years of age inclusive at screening.
Participants who have a confirmed genetic diagnosis of 5q-autosomal recessive SMA
Symptomatic SMA disease, as per investigator's clinical judgement
Participants who are ambulant, where ambulant is defined as able to walk/run 10 meters in <30 seconds at screening
Participants who have received previous SMA disease-modifying therapies may be included provided that:
   Onasemnogene abeparvovec was received at least 90 days prior to screening. Participants should be tapered off steroids prior to receiving risdiplam. In addition, participants should have normal levels of liver function tests, coagulatory parameters, platelets, and troponin-I at 90 days after administration of onasemnogene abeparvovec or at least 1 month after tapering off corticosteroids, whichever comes later.
   Nusinersen last dose was received at least 90 days prior to screening
   Risdiplam is switched to the non-investigational medicinal product (NIMP) provided by the site
Participants who have a legally authorized representative able to consent for the participant as described in Appendix 1, which includes compliance with the requirements and restrictions listed in the Informed Consent Form and in this protocol. Assent must also be given whenever possible
Participants who are able and willing to comply with the study protocol and to complete all study procedures, measurements, and visits
For females of childbearing potential, or who will reach childbearing potential during the study: participants who have a negative blood pregnancy test at screening and agree to remain abstinent (refrain from heterosexual intercourse) or use contraception, as defined below:
Females must remain abstinent or use two methods of contraception, including at least one method with a failure rate of Q 1% per year, during the treatment period and for both 17 months after the final dose of GYM329 and 28 days after the final dose of risdiplam.
A female is considered to be of childbearing potential if she is postmenarchal, has not reached a postmenopausal state (Q 12 continuous months of amenorrhea with no identified cause other than menopause), and is not permanently infertile due to surgery (i.e., removal of ovaries, fallopian tubes, and/or uterus) or another cause as determined by the investigator (e.g., Millerian agenesis). The definition of childbearing potential may be adapted for alignment with local guidelines or regulations.
   Examples of contraceptive methods with a failure rate of <1% per year include bilateral tubal ligation, male sterilization, hormonal contraceptives that inhibit ovulation, hormone-releasing intrauterine devices, and copper intrauterine devices.
   The reliability of sexual abstinence should be evaluated in relation to the duration of the clinical trial and the preferred and usual lifestyle of the individual. Periodic abstinence (e.g., calendar, ovulation, symptothermal, or postovulation methods) and withdrawal are not adequate methods of contraception. If required per local guidelines or regulations, locally recognized adequate methods of contraception and information about the reliability of abstinence will be described in the local Informed Consent Form.
For males who are expected to reach sexual maturity during the study: participants who agree to remain abstinent (refrain from heterosexual intercourse) or use contraceptive methods, and agree to refrain from donating sperm, as defined below:
   With a female partner of childbearing potential who is not pregnant, males must remain abstinent or use a condom plus an additional contraceptive method that together result in a failure rate of Q 1% per year during the treatment period and for 4 months after the final dose of risdiplam or GYM329. Males must refrain from donating sperm during this same period.
   With a pregnant female partner, males must remain abstinent or use a condom during the treatment period and for 28 days after the final dose of risdiplam and 4 months after the last dose of GYM329 to avoid exposing the embryo.

The reliability of sexual abstinence should be evaluated in relation to the duration of the clinical trial and the preferred and usual lifestyle of the individual. Periodic abstinence (e.g., calendar, ovulation, symptothermal, or postovulation methods) and withdrawal are not adequate methods of contraception. If required per local guidelines or regulations, locally recognized adequate methods of contraception and information about the reliability of abstinence will be described in the local Informed Consent Form.

### Exclusion Criteria

Participants are excluded from the study if any of the following criteria apply:
Participants with concomitant or previous participation in any investigational drug or device study within 90 days prior to screening, or 5 half-lives of the drug, whichever is longer, with the exception of those who have completed a risdiplam study, or participated in a nusinersen or onasemnogene abeparvovec study.
Participants who are receiving or have received previous administration of anti-myostatin therapies
For Part 1 participants aged 5-10 years only: Participants who have contraindications for MRI scans (including, but not restricted to, claustrophobia, pacemaker, artificial heart valves, cochlear implants, presence of foreign metal objects in heart or body, including spinal rods, intracranial vascular clips, insulin pumps, etc.), difficulties maintaining a prolonged supine position, or any other clinical history or examination finding that would pose a potential hazard in combination with MRI
Participants with any history of cell therapy
Participants who have been hospitalized for a pulmonary event within the last 2 months or planned hospitalization at the time of screening
Participants who have had surgery for scoliosis or hip fixation in the 6 months preceding screening or planned within the next 9 months (Part 1) or 21 months (Part 2)
Participants who have unstable gastrointestinal, renal, hepatic, endocrine, or cardiovascular system diseases considered to be clinically significant
Participants who have clinically significant ECG abnormalities at screening (e.g., QT interval corrected through use of Bazett's formula [QTcB] Q 460 ms for children up to 10 years, with QTcB used because Bazett's correction is more appropriate in young children) from average of triplicate measurement or cardiovascular disease (e.g., cardiac insufficiency, coronary artery disease, cardiomyopathy, congestive heart failure, family history of congenital long QT syndrome, family history of sudden death) indicating a safety risk for participants
Participants with clinically significant abnormal findings at echocardiography at screening
Participants who have had any major illness within 1 month before screening
Participants who have received any multidrug and toxin extrusion (MATE1/2K) substrates within 2 weeks before screening
Participants who have used any of the following medications within 90 days prior to screening: riluzole, valproic acid, hydroxyurea, sodium phenylbutyrate, butyrate derivatives, creatine, carnitine, growth hormone, anabolic steroids, probenecid, acetyl cholinesterase inhibitors, agents that could potentially increase or decrease muscle strength, and agents with known or presumed histone deacetylase (HDAC) inhibitory effect (participants who are on inhaled corticosteroids, administered either through a nebulizer or an inhaler, will be allowed in the study)
For Part 2 only: Participants who have recently initiated treatment (within <6 months prior to screening) with oral salbutamol or another β2-adrenergic agonist taken orally is not allowed. Participants who have been on oral salbutamol (or another β2-adrenergic agonist) for <6 months before screening and have shown good tolerance are allowed. The dose of β2-adrenergic agonist should remain stable as much as possible for the duration of the study. Use of inhaled β 2-adrenergic agonists (e.g., for the treatment of asthma) is allowed.
Participants who have clinically significant abnormalities in laboratory test results, e.g., ALT values exceeding 1.5□fold the upper limit of normal (ULN), unless the elevated ALT level is considered of muscular origin (i.e., in the absence of other evidence of liver disease) which is supported by elevated creatine kinase and LDH. Out of range creatine kinase levels should be reviewed in light of the underlying SMA pathology of the participant; elevated levels per se do not disqualify the participant from the study. In the case of uncertain or questionable results, tests performed during screening may be repeated before enrollment (Run-in Day −1) to confirm eligibility.
Participants who have ascertained or presumptive hypersensitivity (e.g., anaphylactic reaction) to GYM329 or risdiplam, or to the constituents of their formulations (refer to the Pharmacy Manual)
Participants with concomitant disease or a condition that could interfere with, or treatment of which might interfere with, the conduct of the study, or that would pose an unacceptable risk to the participant in this study
Participants who have a history of any malignancy
Participants who have any clinically relevant history of anaphylactic reaction requiring inotropic support
Participants who have any abnormal skin conditions, pigmentation or lesions in the area intended for SC injection (abdomen) and that would prevent visualization of potential injection site reactions to GYM329
Participants with immobilization, surgical procedures, fracture, or trauma to the upper or lower limbs within 90 days prior to screening
Female participants who are pregnant or breastfeeding, or intending to become pregnant during the study or within either 17 months after the final dose of GYM329 or 28 days after the final dose of risdiplam.
   Females of childbearing potential must have a negative serum pregnancy test result within 14 days prior to enrollment (Run-in Day −1) or baseline (for Part 1 participants not completing an enrollment visit).

### Lifestyle Considerations

### Meals and Dietary Restrictions

This study has no meal or dietary restrictions.

### Activity

Physiotherapy, occupational therapy and other forms of exercise therapy are permitted and frequency and intensity should remain the same during the clinical study.

### Contraception Requirements

During the study, participants who have reached puberty must use contraception or take other precautions.

### Study Treatments Administered

In this protocol, “study treatment” refers to all treatments assigned to participants as part of this study (i.e., blinded and open-label GYM329, blinded GYM329-matched placebo, and open-label risdiplam). Table 6 provides a description of the study treatments for this study.

TABLE 6
Study Treatment Description

| | Blinded and Unblinded | Blinded RO7204239- | |
|---|---|---|---|
| | RO7204239 | Matched Placebo | Risdiplam |
| Use | Experimental in | Placebo-comparator | Active in combination |
| | combination with | in combination with | with blinded and |
| | active risdiplam | active risdiplam | unblinded experimental |
| | | | RO7204239 or matched |
| | | | placebo-comparator and |
| | | | as monotherapy in the |
| | | | run-in period |
| Type of | IMP | IMP | NIMP |
| medicinal | | | |
| product | | | |
| Drug form | 1.0 mL of sterile, | 1.0 mL of sterile, | powder for constitution |
| | preservative-free | preservative-free | to an oral solution |
| | solution for injection | solution for | |
| | containing 80 mg of | injections | |
| | RQ7204239 | | |
| Unit Dose | 80 mg/mL | Not applicable | 60 mg constituted to |
| Strength(s) | | | yield an oral solution |
| | | | containing 0.75 mg/mL |
| Dosage(s) | Low-dose cohort: 7.4 mg | Not applicable | 5 mg QD (participants ≥ |
| | Q4W (participants < 20 | | 20 kg) or 0.25 mg/kg QD |
| | kg) or 10.6 mg Q4W | | (participants < 20 kg) |
| | (participants ≥ 20 kg) | | |
| | High-dose cohort: 24 mg | | |
| | Q4W (participants < 20 | | |
| | kg) or 36 mg Q4W | | |
| | (participants ≥ 20 kg); | | |
| | final dose TBD based on | | |
| | emerging data | | |
| Formulation(s) | Refer to Investigator's | Not applicable | Refer to Investigator's |
| | Brochure | | Brochure |
| Packaging | 3-mL glass vials | 3-mL glass vials | 100-mL amber glass |
| | | | bottle ^{a} |
| Route of | SC Injection | SC injection | Oral |
| administration | | | |
| Source | Sponsor ^{b} | Sponsor ^{b} | Sponsor ^{b} |

IMP = investigational medicinal product; NIMP = non-investigational medicinal product; Piba = press in bottle adapter; Q4W = every 4 weeks; QD = once a day; TBD = to be determined.
^{a} Risdiplam powder for an oral solution in a 100 mL amber glass bottle plus Piba (press in bottle adapter) and Enfit syringe for administration of the oral solution.
^{b} Diluent will also be supplied by the Sponsor.

### GYM329

GYM329 will be provided in 3 mL glass vials containing 80 mg/mL and must be prepared for dosing under appropriate aseptic conditions. The solution must be diluted as necessary and filtered prior to injection using a needle filter. The solution ready for injection should preferably be used immediately. Detailed instructions are provided in the Pharmacy Manual.

GYM329 will be administered every 4 weeks by SC injection in the abdomen. The administration volume in Part 1 will range from 0.3-0.5 mL depending on the dose (see Table 5). Each injection should be administered in a separate location in rotating quadrants of the abdomen at each study visit where this treatment is administered. GYM329 will be administered at the clinical site by site staff. GYM329 will be administered after all pre-dose assessments have been conducted and criteria for temporarily delaying administration have been reviewed (see Section 6.6). Participants will be monitored at the site for a minimum of 6 hours after the first 2 administrations, and for 2 hours for administrations thereafter (or longer if deemed necessary by the investigator/site staff).

Only participants enrolled in the study may receive GYM329, only authorized staff may supply GYM329, and only authorized staff or trained study personnel may administer the study drug.

Any overdose or incorrect administration of GYM329 should be noted on the Study Drug Administration electronic Case Report Form (eCRF). Adverse events associated with an overdose or incorrect administration of GYM329 should be recorded on the Adverse Event eCRF.

Accurate records of GYM329 received at, dispensed from, and disposed of by the study site should be recorded on the drug accountability log.

Refer to the Pharmacy Manual and the GYM329 Investigator's Brochure for information on GYM329 handling, including preparation and storage, and accountability.

Guidelines for medical management of local injection reactions and systemic injection reactions are provided in Appendix 5. The use of medication to treat these events must be recorded as concomitant medication in the eCRF.

### Risdiplam

Risdiplam is supplied as a powder for constitution to an oral solution; each bottle contains 60 mg of risdiplam which is constituted with purified water or water for injection to yield an oral solution containing 0.75 mg/mL of risdiplam.

All participants in this study will receive risdiplam for the duration of their participation in the treatment periods of the study. Risdiplam will be administered at a dose of 5 mg once daily for participants with a BW ≥20 kg or 0.25 mg/kg for participants with a BW <20 kg, using the re-usable oral syringe provided. The Sponsor will provide oral syringes for the participant/caregiver to administer the solution.

The first dose of risdiplam will be administered at the clinical site after all pre-dose assessments have been conducted. Throughout the study, risdiplam should be taken orally once daily at home after the morning meal at approximately the same time each day.

On the days of site visits when risdiplam PK sampling is planned, risdiplam will be administered at the clinical site to allow for pre- and post-dose blood sampling (see Section 1.3). On these days, participants should have their regular morning meal at home before coming to the site; should there be a long time between this meal and risdiplam administration, a snack will be given to the participant by the site prior to risdiplam administration.

The participant should drink water after taking risdiplam to ensure risdiplam has been completely swallowed. If risdiplam gets on the skin, the area should be washed with soap and water.

Risdiplam should be taken immediately after it is drawn up into the oral syringe. If it is not taken within 5 minutes, it should be discarded from the oral syringe and a new dose should be prepared.

Only participants enrolled in the study may receive risdiplam, only authorized staff may supply risdiplam, and only authorized staff, trained study personnel, or trained participants/caregivers may administer risdiplam.

Any overdose or incorrect administration of risdiplam should be noted on the Study Drug Administration eCRF. Adverse events associated with an overdose or incorrect administration of risdiplam should be recorded on the Adverse Event eCRF.

Accurate records of risdiplam received at, dispensed from, and disposed of by the study site should be recorded on the drug accountability log.

Refer to the Pharmacy Manual and risdiplam Investigator's Brochure for information on risdiplam handling, including preparation and storage, and accountability.

### Placebo

Placebo of identical appearance, composition (except GYM329) and identical volume to GYM329 will be administered by SC injection to all participants randomized to placebo+risdiplam, and will be administered in the same dose regimen (every 4 weeks).

### Concomitant Therapy

Any concomitant medication and/or vaccine, including over-the-counter or prescription medicines, vitamins, and/or herbal supplements from 30 days prior to study screening to the study completion or early discontinuation visit must be reported to the investigator and recorded on the “Concomitant Medications” eCRF with the following information:
Reason for use
Dates of administration, including start and end dates
Dosage information, including dose and frequency

Any non-medication interventions (e.g., individual psychotherapy, cognitive behavioral therapy, physical therapy and rehabilitative therapy) used by a participant in addition to protocol-mandated treatment from 30 days prior to study screening to the study completion or early discontinuation visit must be reported to the investigator and recorded on the “Non-Pharmacological Interventions” eCRF.

The Medical Monitor should be contacted if there are any questions regarding concomitant or prior therapy.

### Permitted Therapy

Examples of allowed medications include the following (unless prohibited in below):
Treatment with oral salbutamol or another 02-adrenergic agonist taken orally is allowed as long as treatment has been introduced for at least 6 months before screening and participant has shown good tolerance
Use of inhaled D2-adrenergic agonists (e.g., for the treatment of asthma)
Inhaled corticosteroids
Other inhaled drugs for obstructive airways diseases (e.g., anticholinergics and anti-allergic agents)
Other systemic drugs for obstructive airways diseases (e.g., leukotriene receptor antagonists)
Laxatives and other drugs for functional gastrointestinal disorders
Analgesics, including opioids (e.g., hydromorphone or codeine)
Antibiotics (with the exceptions mentioned below)
Antihistamines
Proton pump inhibitors
Any drugs required to treat local or systemic injection reactions
The use of topical analgesia at the sites of GYM329 SC injection may be offered to all participants as per local guidelines

Unless specified differently below, for any chronic treatment (defined as treatment for a minimum of 8 weeks), participants should be on a stable regimen for 6 weeks prior to screening and should remain on a stable regimen throughout the double-blind periods of the study (weight/age related dose adjustments of chronic treatments are allowed)

### Prohibited Therapy

All medications (prescription and over-the-counter [OTC]) taken within 30 days of study screening will be recorded on the appropriate eCRF.

Any administration of nusinersen, either in a clinical study or for medical care, within 90 days prior to screening and throughout the study is explicitly prohibited.

MATE1/2K substrates are explicitly prohibited for 2 weeks prior to screening and throughout the study.

Use of the following therapies is prohibited during the study and for at least 90 days prior to screening:
Riluzole
Valproic acid
Hydroxyurea
Sodium phenylbutyrate
Butyrate derivatives
Creatine
Carnitine
Growth hormone
Anabolic steroids
Probenecid
Acetyl cholinesterase inhibitors
Chronic oral or parenteral use of corticosteroids (inhaled corticosteroid use is
allowed) unless required to manage injection reactions
Agents anticipated to increase or decrease muscle strength or agents with known or presumed HDAC inhibition activity

1-86. (canceled)

87. A method for treating spinal muscular atrophy (SMA) in a human in need thereof, the method comprising:
administering risdiplam to the human; and
administering an antibody that binds to myostatin to the human;
wherein the antibody comprises six complementary determining regions (CDRs) CDRH1, CDRH2, CDRH3, CDRL1, CDRL2 and CDRL3, wherein:
CDRH1 is SEQ ID 1;
CDRH2 is SEQ ID 2;
CDRH3 is SEQ ID 3;
CDRL1 is SEQ ID 4;
CDRL2 is SEQ ID 5; and
CDRL3 is SEQ ID 6.

88. The method of claim 87, wherein antibody comprises a heavy chain variable region (VH) having at least 90% sequence identity to the amino acid sequence of SEQ ID 7, and a light chain variable region (VL) having at least 90% sequence identity to the amino acid sequence of SEQ ID 8.

89. The method of claim 87, wherein antibody comprises a VH comprising the amino acid sequence of SEQ ID 7, and a VL comprising the amino acid sequence of SEQ ID 8.

90. The method of claim 87, wherein the antibody comprises a heavy chain region comprising the amino acid sequence of SEQ ID 9, and a light chain region comprising the amino acid sequence of SEQ ID 10.

91. The method of claim 87, wherein the antibody is GYM329.

92. The method of claim 87, wherein the antibody inhibits activation of myostatin.

93. The method of claim 87, wherein the antibody blocks the proteolytic release of mature myostatin.

94. The method of claim 87, wherein the human is administered risdiplam for at least 4 weeks before the first dose of the antibody is administered to the human.

95. The method of claim 87, wherein the human is administered risdiplam for at least 8 weeks before the first dose of the antibody is administered to the human.

96. The method of claim 87, wherein the antibody is administered every four weeks.

97. The method of claim 87, wherein the human has type I SMA, type II SMA, or type III SMA.

98. The method of claim 87, wherein:
the human has type I SMA, type II SMA, or type III SMA;
the human is administered risdiplam for at least 8 weeks before the first dose of the antibody is administered to the human;
the antibody is administered every four weeks; and
the antibody comprises a heavy chain variable region (VH) having at least 90% sequence identity to the amino acid sequence of SEQ ID 7, and a light chain variable region (VL) having at least 90% sequence identity to the amino acid sequence of SEQ ID 8.

99. The method of claim 98, wherein antibody comprises a VH comprising the amino acid sequence of SEQ ID 7, and a VL comprising the amino acid sequence of SEQ ID 8.

100. The method of claim 98, wherein the antibody comprises a heavy chain region comprising the amino acid sequence of SEQ ID 9, and a light chain region comprising the amino acid sequence of SEQ ID 10.

101. A method for the treatment of SMA in a human in need thereof, the method comprising:
a) administering risdiplam to the human for at least 2 weeks; then
b) administering risdiplam and an anti-myostatin antibody to the human, wherein the antibody comprises six complementary determining regions (CDRs) CDRH1, CDRH2, CDRH3, CDRL1, CDRL2 and CDRL3, wherein:
CDRH1 is SEQ ID 1;
CDRH2 is SEQ ID 2;
CDRH3 is SEQ ID 3;
CDRL1 is SEQ ID 4;
CDRL2 is SEQ ID 5; and
CDRL3 is SEQ ID 6.

102. The method of claim 101, wherein the antibody is administered every four weeks.

103. The method of claim 101, wherein antibody comprises a heavy chain variable region (VH) having at least 90% sequence identity to the amino acid sequence of SEQ ID 7, and a light chain variable region (VL) having at least 90% sequence identity to the amino acid sequence of SEQ ID 8.

104. The method of claim 101, wherein antibody comprises a VH comprising the amino acid sequence of SEQ ID 7, and a VL comprising the amino acid sequence of SEQ ID 8.

105. The method of claim 101, wherein the antibody comprises a heavy chain region comprising the amino acid sequence of SEQ ID 9, and a light chain region comprising the amino

106. The method of claim 101, wherein the antibody is GYM329.

107. A method for the treatment of SMA in a human in need thereof, the method comprising orally administering risdiplam once per day to the human and subcutaneously administering GYM329 every four weeks to the human.

108. The method of claim 107, wherein risdiplam is administered orally once per day to the human for at least 2 weeks prior to the first dose of GYM329.

109. The method of claim 107, wherein the human is older than 2 years and with a body weight of less than 20 kg, and:
risdiplam is orally administered once per day at a dose of 0.25 mg/kg; and
GYM329 is subcutaneously administered every 4 weeks at a dose of 7.4 mg.

110. The method of claim 107, wherein the human is older than 2 years and with a body weight of less than 20 kg, and:
risdiplam is orally administered once per day at a dose of 0.25 mg/kg; and
GYM329 is subcutaneously administered every 4 weeks at a dose of 24 mg.

111. The method of claim 107, wherein the human is has a body weight of greater than or equal to 20 kg, and:
risdiplam is orally administered once per day at a dose of 5 mg; and
GYM329 is subcutaneously administered every 4 weeks at a dose of 10.6 mg.

112. The method of claim 107, wherein the human is has a body weight of greater than or equal to 20 kg, and:
risdiplam is orally administered once per day at a dose of 5 mg; and
GYM329 is subcutaneously administered every 4 weeks at a dose of 36 mg.
